(19) United States
(12) Patent Application Publication
(10) Pub. No.: US 2010/0286238 A1
(43) Pub. Date: Nov. 11, 2010

(54) SUPPRESSION OF VIRUSES INVOLVED IN RESPIRATORY INFECTION OR DISEASE

(76) Inventors: Laurent Pierre Rivory, New South Wales (AU); Michael Poidinger, New South Wales (AU); Donald John Birkett, New South Wales (AU); Gregory Martin Arndt, New South Wales (AU); Toby Passioura, New South Wales (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/598,819

(22) PCT Filed: May 14, 2008
(86) PCT No.: PCT/AU08/00680

§ 371 Date: Apr. 21, 2010

Related U.S. Application Data
(60) Provisional application No. 60/938,052, filed on May 15, 2007.

(51) Int. Cl. A61K 31/7105; C07H 21/02; C12N 15/79; C12N 5/10; A61P 31/12; A61P 11/00

(52) U.S. Cl. 514/44 A; 536/24.5; 435/320.1; 435/325

(57) ABSTRACT

The present invention concerns methods and reagents useful in decreasing the level of or severity of respiratory infection or disease due to paramyxoviruses, such as RSV or HPIV, or coronavirus infection. Particularly, the invention relates to modulating gene expression using a multitargeting interfering RNA molecules that target multiple target sites on one or more pre-selected RNA molecules.

### FIELD OF THE INVENTION

The present invention concerns methods and reagents useful in decreasing the level of or severity of respiratory infection or disease due to paramyxoviruses, such as RSV or HPIV, or coronavirus infection. Particularly, the invention relates to modulating gene expression using a multitargeting interfering RNA molecules that target multiple target sites on one or more pre-selected RNA molecules.

### BACKGROUND OF THE INVENTION

RNA interference (RNAi) is a diverse, evolutionarily conserved mechanism in eukaryotic cells, which inhibits the transcription and translation of target genes in a sequence-specific manner. It is now known that single and double-stranded RNA can modulate expression of or modify processing of target RNA molecules by a number of mechanisms. Some such mechanisms tolerate variation in the amount of sequence complementarity required between the modulatory (or interfering) RNA and the target RNA. Certain microRNAs can translationally repress target mRNA having as little as 6 nucleotides of complementarity with the microRNA. The development of RNA interfering agents, for example, using double-stranded RNA to repress expression of disease-related genes is currently an area of intense research activity.

Double-stranded RNA of 19-23 bases in length is recognized by an RNA interference silencing complex (RISC) into which an effector strand (or “guide strand”) of the RNA is loaded. This guide strand acts as a template for the recognition and destruction of highly complementary sequences present in the transcriptome. Alternatively, through the recognition and binding of RNA sequences of lower complementarity, interfering RNAs may induce translational repression without mRNA degradation. Such translational repression appears to be a mechanism of action of endogenous microRNAs, a group of short non-coding RNAs involved in differentiation and development.

Efforts at implementing interfering RNAs therapeutically thus far have mostly focused on producing specific double stranded RNAs, each with complete complementarity to a particular target transcript. Such double-stranded RNAs (dsRNAs) are potentially effective where a single suitable target can be identified, however, dsRNAs, particularly those designed against one target, may have at least two categories of off-target side effects that need to be avoided or minimized. Undesirable side effects can arise through the triggering of innate immune response pathways (e.g. Toll-like Receptor 3, 7, and 8, and the so-called interferon response) and through inadvertent inhibition of protein expression from related or unrelated transcripts (either by RNA degradation, translational repression or other mechanisms). Some bioinformatic and/or experimental approaches have been developed to try to minimize off-target effects. Algorithms for in silico hybridization are known, and others have been developed for predicting target accessibility and loading bias in an effort to eliminate or minimize side-effects while maintaining effectiveness.

Therapeutic approaches to viral infections continue to be major challenges in agriculture, as well as in animal and human health. The nature of the replication of viruses makes them highly plastic, “moving targets” therapeutically—capable of altering structure, infectivity, and host profile. The recent emergence of viruses such as Severe Acute Respiratory Syndrome (“SARS”) and Avian Influenza Virus (“bird flu”) exemplify these challenges. Even well-described viruses such as those involved in Acquired Immunodeficiency Syndrome or AIDS (e.g. Human Immunodeficiency Viruses, HIV-1 and HIV-2), continue to defy efforts at treatment and vaccination because of on-going viral mutation and evolution.

Furthermore, although nucleic acid therapeutics such as interfering RNAs are candidates for viral therapy, in part because modern rapid gene sequencing techniques allow viral genome sequences to be determined even before any encoded functions can be assessed, the error-prone replication of viruses, particularly RNA viruses, and development of resistance which can arise during treatment, means that substantial genomic diversity can arise rapidly in an infected population. Thus far, strategies for the development of nucleic acid therapeutics have largely centered on the targeting of highly-conserved regions of the viral genome. It is unclear whether these constructs are efficient at treating viral infection or preventing emergence of resistant viral clones.

The Family Paramyxoviridae, includes the human respiratory disease viruses Human Respiratory Syncytial Virus (HRSV) types A and B, and Human Parainfluenza Viruses (HPIV, types 1, 2, 3, 4a and 4b) as well as the measles virus and mumps virus.

The paramyxoviridae viruses contain a viral genome that is a single, linear, negative-sense (anti-mRNA sense) RNA molecule, 15-20 kb in length, that is coated with the nucleocapsid protein N (alternatively designated NP). This nucleocapsid protein is associated with the two RNA-dependent RNA polymerase (RdRP) subunits, namely, the large viral protein L and the accessory phosphoprotein P. The resultant ribo-nucleoprotein complex that contains the RNA synthesis machinery is finally packaged inside the structural shell of the virion, mainly made up of glycoproteins. The outer membrane contains the matrix (M) protein and 2 transmembrane envelope proteins, namely, fusion protein (F), and a second attachment protein that varies amongst different members of the family, designated HN (with haemaglutinin-neuraminidase activity, found in parainfluenza types 1-4), H (haemaglutinin activity only, found in mumps) or G (neither activity, found in RSV). The genera pneumovirus of this family, of which RSV is a member, contains the G protein mentioned previously, and also contains 4 extra proteins, two structural—the small hydrophobic protein (SH) and a second matrix protein (M2), and 2 non structural, designated NS1 and NS2. All the protein components of the virion, are encoded by the viral genome. mRNAs corresponding to each individual gene are transcribed by a stop-restart mechanism and contain features of standard eukaryotic mRNAs in that they are 5′-capped and 3′-polyadenylated. The transcription process exhibits “polarity,” such that the genes most proximal to the promoter (3′ end of the negative-strand genome) are transcribed most abundantly.

Respiratory syncytial virus (RSV) accounts for the majority of acute lower respiratory tract infections which can lead to bronchiolitis and pneumonia in infants worldwide. Disease severity has been found to be strongly associated with the infants' inflammatory response, specifically interleukin-8 (IL-8) production in the airways. The elderly and immunosuppressed transplant patients are also susceptible to infection with RSV, often resulting in pneumonia. There are 2 major groups of RSV (A & B) which each comprise a large number of strains.

Human parainfluenza viruses (HPIV) are causative agents of respiratory tract infections leading to pharyngitis, bronchitis, bronchiolitis, croup and pneumonia in children less than five years of age. There are four main types of HPIV, with each type comprising a number of different strains.

Disease caused by the measles virus is typically marked by a prodrome of fever, conjunctivitis, coryza, and cough followed by the development of a rash. The two most serious complications of measles infection are acute postinfectious and subacute sclerosing panencephalitis (SSPE).

Infection with mumps virus often results in a minor illness characterized by inflammation of the salivary glands, and rarely encephalitis. Mumps infection in adult males can result in orchitis, or inflammation of the testes, resulting in destruction of the testicular tissue.

The Family Coronaviridae, consists of 2 genera including coronavirus and torovirus. There are 3 main groups of Coronaviruses. Group 1 consists of Human Coronavirus 229E and NL63 as well as transmissible gastroenteritis virus. Group 2 consists of Human Coronavirus OC43 and HKU1 as well as the Coronavirus responsible for SARS. Coronaviruses cause the common cold, SARS, gastroenteritis, and in some cases neurological syndromes.

The coronaviruses consist of a single, positive sense RNA genome of 27-31 kb and this RNA is packaged with a phosphoprotein (N). The genome is encapsulated in a lipid envelope, which contains the S (spike), M (membrane) and HE (haemagglutinin-esterase) glycoproteins. The genome also encodes a further 10-15 nonstructural proteins. Upon infection the positive sense genome is translated to produce a viral polymerase which is able to produce the negative strand. From this negative strand a set of transcripts is produced, which all contain an identical 5′ non-translated leader sequence and 3′ polyadenylated ends.

Interfering RNA molecules with specificity for multiple binding sequences present in distinct genetic contexts in one or more pre-selected target RNA molecules are described in co-pending international patent application nos. PCT/AU2006/001741 and PCT/AU2006/001750, the disclosures of which are incorporated herein in their entirety. The present inventors have now shown that interfering RNA molecules with multiple targets are useful in decreasing the level of or severity of a paramyxovirus infection such as RSV.

### SUMMARY OF THE INVENTION

In a first embodiment, this invention relates to a multitargeting interfering RNA molecule comprising a guide strand of the Formula (I):

5′-p-XSY-3′

wherein p consists of a terminal phosphate group that is independently present or absent;wherein S consists of a first nucleotide sequence of a length of about 5 to about 20 nucleotides that is at least partially complementary to a first portion of each of at least two binding sequences present in distinct genetic contexts in one or more pre-selected target RNA molecules; wherein X is absent or consists of a second nucleotide sequence; wherein Y is absent or consists of a third nucleotide sequence, provided that X and Y are not absent simultaneously; wherein XSY is at least partially complementary to each of said binding sequences to allow a stable interaction therewith and wherein at least one of the binding sequences is present in paramyxovirus RNA or coronavirus, other than SARS, RNA. Preferably S is completely complementary to the first portion of each of at least two binding sequences and also preferably, the first portion of each of at least two binding sequences is a seed sequence. In some embodiments, X can consist of one, two or more nucleotides and Y can independently be at least partially complementary to a second portion of each of the binding sequences, said second portion is adjacent to and connected with the 5′-end of said first portion of the binding sequences. Also preferably, S is of a length of about 8 to about 15 nucleotides. XSY is preferably of a length of about 17 to about 25 nucleotides. Preferably, the multitargeting interfering RNA molecules of this invention further comprise a passenger strand that is at least partially complementary to the guide strand to allow formation of a stable duplex between the passenger strand and the guide strand and these RNA molecules preferably include one or more terminal overhangs and these overhangs preferably are between 1 to 5 nucleotides. Preferably the passenger strand and the guide strand are completely complementary to each other. It is possible for the multitargeting interfering RNA molecules of this invention to target binding sequences present in distinct genetic contexts in one or alternatively in at least 2 pre-selected target RNA molecules. At least one of the pre-selected target RNA molecules may be a non-coding RNA molecule. At least one of the pre-selected target RNA molecules may be a messenger RNA molecule. Also, in the multitargeting interfering RNA molecules of this invention, at least one of the binding sequences may be present in the 3′-untranslated region (3′UTR) of a messenger RNA molecule.

In an alternative embodiment this invention relates to a multitargeting interfering RNA molecule comprising Formula (II):

5′-p-A B C-3′
3′-A′B′C′-p-5′

wherein p consists of a terminal phosphate group that is independently present or absent; wherein B consists of a first nucleotide sequence of a length of about 5 to about 20 nucleotides that is partially, preferably completely, complementary to a first portion of a first binding sequence, and B′ consists of a second nucleotide sequence of a length of about 5 to about 20 nucleotides that is partially, preferably completely, complementary to a first portion of a second binding sequence, wherein said first and second binding sequences are present in distinct genetic contexts in at least one pre-selected target RNA molecule, and wherein B and if are at least substantially complementary to each other but are not palindromic; and further wherein A, A′, C, or C′, is independently absent or consists of a nucleotide sequence; wherein ABC is at least partially complementary to the first binding sequence to allow stable interaction therewith; and wherein C′B′A′ is at least partially complementary to the second binding sequence to allow stable interaction therewith and is at least partially complementary to ABC to form a stable duplex therewith and wherein at least one of the first or second binding sequences is present in paramyxovirus RNA or coronavirus, other than SARS, RNA.

In one version of this embodiment, A, A′, C, or C′, independently consists of one or more nucleotides and in another aspect of this embodiment A consists of a third nucleotide sequence that is at least partially complementary to a second portion of the first binding sequence, where the second portion is adjacent to and connected with the 3′-end of said first portion of the first binding sequence, and where A′ consists of a fourth nucleotide sequence that is substantially complementary to the third nucleotide sequence. Preferably in this aspect A and N are completely complementary to each other. It is also preferred that A is completely complementary to the second portion of the first binding sequence.

In another aspect of this embodiment, C′ is designed to consist of a fifth nucleotide sequence that is at least partially complementary to a second portion of the second binding sequence and the second portion is adjacent to and connected with the 3′-end of said first portion of the second binding sequence. In this aspect C consists of a sixth nucleotide sequence that is substantially complementary to the fifth nucleotide sequence. Preferably C and C′ are completely complementary to each other. It is also preferred that C′ is completely complementary to the second portion of the second binding sequence.

In yet other aspects of this preferred embodiment, B and B′ are completely complementary to each other. It is also preferred that AB is completely complementary to the first portion and the second portion of the first binding sequence. It is also contemplated that C′B′ is completely complementary to the first portion and the second portion of the second binding sequence. Further, ABC and C′B′A′ can be completely complementary to each other. Optionally, in aspects of this invention, B consists of a first nucleotide sequence of a length of about 8 to about 15 nucleotides and ABC and C′B′A′ preferably include lengths of about 15 to about 29 nucleotides. Also preferably, each of ABC and C′B′A′ are of a length of about 19 to about 23 nucleotides. In some aspects of this embodiment, the multitargeting interfering RNA molecule comprises one or more terminal overhangs and preferably these overhangs consist of 1 to 5 nucleotides.

In yet a further aspect of this embodiment, the first and the second binding sequences of the multitargeting interfering RNA molecule are present in distinct genetic contexts in one pre-selected target RNA molecule or alternatively, the first and the second binding sequences are present in distinct genetic contexts in at least two pre-selected target RNA molecules. At least one of the pre-selected target RNA molecules may be a non-coding RNA molecule. At least one of the pre-selected target RNA molecules may be a messenger RNA (mRNA). In a further embodiment at least one of the binding sequences may be present in the 3′-untranslated region (3′UTR) of a mRNA molecule.

In another aspect the first and second binding sequences of the multitargeting interfering RNA molecule are in one virus or are within two different viruses.

In an embodiment of the invention the multitargeting interfering RNA targets virus RNA and host RNA which encodes a protein involved in the infection process. Examples of host proteins involved in RSV infection include proteins involved in inflammation such as IL-8, receptors to which RSV can bind such as heparan sulphate, GTP-binding proteins such as RhoA, cytoskeletal proteins such as actin or profilin and heat shock proteins such as Hsp70 (Sugrue, 2006).

If targeting HPIV along with a host protein, host proteins that could be targeted for knockdown, thereby reducing the replication or survival of the virus include proteins involved in heparan sulfate synthesis such as heparan sulfate synthase, cytoskeletal proteins such as actin, sialylglycoprotein cellular receptors and protein synthesis and folding proteins such as Hsp90.

Host proteins that could be targeted for knockdown in treating or suppressing coronavirus infection, and in doing so, reduce the replication or survival of the virus include cellular receptors such as angiotensin converting enzyme (ACE2), human aminopeptidase N, receptor glycoproteins and HLA class I antigens as well as proteins involved in signal transduction including MEK1/2 or ERF1/2.

In an embodiment the multitargetting interfering RNA targets RSV and IL-8 and targets a sequence selected from the group consisting of ACAAACUUUC (SEQ ID NO: 6), AACCAUCUCACU (SEQ ID NO: 7), CAUAAAGACAU (SEQ ID NO: 86), UUAUCAAAGAA (SEQ ID NO: 1), AUUGAAUGG, GAACUGAGA, GUGAUAUUUG (SEQ ID NO: 87), UGUGGUAUC, UCAAGCAAAU (SEQ ID NO: 88), CAGAUGCAA, AUACAAGAU, UUCCUGGUUA (SEQ ID NO: 89), AUCCAGAAC, AUAUAAGGAUU (SEQ ID NO: 90), UAGCAAAAUUG (SEQ ID NO: 91), CAUCAUAACA (SEQ ID NO: 92), AAUUUAGCUGGA (SEQ ID NO: 2), GGAAGCACU, AUAAAUUUCAA (SEQ ID NO: 93), CAUCAAAUAU (SEQ ID NO: 3), GAUUGAAUA, AUAGUUAUA, UUAUUAGAUAA (SEQ ID NO: 4), UUAGAUAAAU (SEQ ID NO: 94), AUUUCAAUCA (SEQ ID NO: 95), UUGAUACUCC (SEQ ID NO: 5), ACUAACAAU, UCCUAGUUU, AGUUUGAUAC (SEQ ID NO: 96), AUUGCCAGC, GAAUAAUGA, ACAGCCAAA, AUUAGUAAU, UUUAUUAUGU (SEQ ID NO: 97), CAAAUAGAU, AAUAGAUUC, AUAAUAUUAU (SEQ ID NO: 98), AUAUGAAAC, AGGACAAGA, UACAUUAUU and CUCUGUGGU.

In another embodiment the multitargetting interfering RNA targets two sequences within RSV and targets a sequence selected from the group consisting of AAAGUUUGCU (SEQ ID NO: 99), AGAAGAUGC, AGAUAGUAU, UAUUGAUAC, AAAGAUCCCAA (SEQ ID NO: 100), AGUAUCAUA, UCAAUAGAUAUA (SEQ ID NO: 101), CCCUAUAACA (SEQ ID NO: 102), CAGAUGAUA, UAUCAUGUA, CUAAACUAUA (SEQ ID NO: 66), AAUCCAACA, AUCAACAUUGA (SEQ ID NO: 103), CGAUAAUAUAA (SEQ ID NO: 67), ACAUUAGUA, UGUAUAGCA, UAGAAGCUAU (SEQ ID NO: 104), UUUUUGUUCA (SEQ ID NO: 105), AUUGAACAACC (SEQ ID NO: 106), AUCAUCCAAC (SEQ ID NO: 107), UUGACUCAAU (SEQ ID NO: 108), UCAAGAUCU and AGAGGCUAU.

In another embodiment the multitargetting interfering RNA targets RSV and HPIV and targets a sequence selected from the group consisting of AGAAUCAAUAAAGG (SEQ ID NO: 109), AAAGAAGACCCUA (SEQ ID NO: 110), and UGAUGAAAAAUU (SEQ ID NO: 111).

In other aspects of this invention, S consists essentially of a nucleotide sequence selected from the group consisting of
(i) GAAAGUUUGU (SEQ ID NO: 112), AGUGAGAUGGUU (SEQ ID NO: 113), AUGUCUUUAUG (SEQ ID NO: 114), UUCUUUGAUAA (SEQ ID NO: 115), CCAUUCAAU, UCUCAGUUC, CAAAUAUCAC (SEQ ID NO: 116), GAUACCACA, AUUUGCUUGA (SEQ ID NO: 117), UUGCAUCUG, AUCUUGUAU, UAACCAGGAA (SEQ ID NO: 118), GUUCUGGAU, AAUCCUUAUAU (SEQ ID NO: 119), CAAUUUUGCUA (SEQ ID NO: 120), UGUUAUGAUG (SEQ ID NO: 121), UCCAGCUAAAUU (SEQ ID NO: 122), AGUGCUUCC, UUGAAAUUUAU (SEQ ID NO: 123), AUAUUUGAUG (SEQ ID NO: 124), UAUUCAAUC, UAUAACUAU, UUAUCUAAUAA (SEQ ID NO: 125), AUUUAUCUAA (SEQ ID NO: 126), UGAUUGAAAU (SEQ ID NO: 127), GGAGUAUCAA (SEQ ID NO: 128), AUUGUUAGU, AAACUAGGA, GUAUCAAACU (SEQ ID NO: 129), GCUGGCAAU, UCAUUAUUC, UUUGGCUGU, AUUACUAAU, ACAUAAUAAA (SEQ ID NO: 130), AUCUAUUUG, GAAUCUAUU, AUAAUAUUAU (SEQ ID NO: 98), GUUUCAUAU, UCUUGUCCU, AAUAAUGUA and ACCACAGAG which target RSV and IL-8; or
(ii) AGCAAACUUU (SEQ ID NO: 131), CGAUCUUCU, AUACUAUCU, GUAUCAAUA, UUGGGAUCUUU (SEQ ID NO: 132), UAUGAUACU, UAUAUCUAUUGA (SEQ ID NO: 133), UGUUAUAGGG (SEQ ID NO: 134), UAUCAUCUG, UACAUGAUA, UAUAGUUUAG (SEQ ID NO: 135), UGUUGGAUU, UCAAUGUUGAU (SEQ ID NO: 136), UUAUAUUAUCG (SEQ ID NO: 137), UACUAAUGU, UGCUAUACA, AUAGCUUCUA (SEQ ID NO: 138), UGAACAAAAA (SEQ ID NO: 139), GGUUGUUCAAU (SEQ ID NO: 140), GUUGGAUGAU (SEQ ID NO: 141), AUUGAGUCAA (SEQ ID NO: 142), AGAUCUUGA and AUAGCCUCU which multitarget RSV; or
(iii) CCUUUAUUGAUUCU (SEQ ID NO: 143), UAGGGUCUUCUUU (SEQ ID NO: 144) and AAUUUUUCAUCA (SEQ ID NO: 145), which target RSV and parainfluenza virus.

In another aspects the multitargetting interfering RNA molecule comprises a duplex selected from the group consisting of

| | |
|---|---|
| CCCCAAUAUUAUCAAAGAAUU | (SEQ ID NO: 37) |
| GUGGGGUUAUAAUAGUUUCUU | (SEQ ID NO: 8) |
| ACCCAUUCAGUGUGGUAUUUU | (SEQ ID NO: 39) |
| UUUGGGUAAGUCACACCAUAG | (SEQ ID NO: 10) |
| GGUUCGCAGAUGCAACCAAUU | (SEQ ID NO: 40) |
| UCCCAAGCGUCUACGUUGGUU | (SEQ ID NO: 11) |
| ACCAUGAAUAAUCCAGAAUUU | (SEQ ID NO: 41) |
| CAUGGUACUUAUUAGGUCUUG | (SEQ ID NO: 12) |
| CCAUGAAUAAUCCAGAAUAUU | (SEQ ID NO: 42) |
| AUGGUACUUAUUAGGUCUUGU | (SEQ ID NO: 13) |
| GUCAAAUUUAGCUGGAAAUUU | (SEQ ID NO: 43) |
| UUCAGUUUAAAUCGACCUUUA | (SEQ ID NO: 14) |
| CUUAUUUAUCCAUCAAAUAUU | (SEQ ID NO: 44) |
| AUGAAUAAAUAGGUAGUUUAU | (SEQ ID NO: 15) |
| UGAUGAAUUAUUAGAUAAAUU | (SEQ ID NO: 46) |
| UUACUACUUAAUAAUCUAUUU | (SEQ ID NO: 17) |
| UAGAUUUGAUACUCCUAAUUU | (SEQ ID NO: 47) |
| CUAUCUAAACUAUGAGGGUUA | (SEQ ID NO: 18) |
| GAAUUAGCGAAUAAUGAAUUU | (SEQ ID NO: 48) |
| AACUUAAUCGCUUAUUACUUA | (SEQ ID NO: 19) |
| CACAGUCAUAAUUAGUAAUUU | (SEQ ID NO: 49) |
| AGGUGUCAGUAUUAAUCAUUA | (SEQ ID NO: 20) |
| GCCCAAAUUUAUCAAAGAAUU | (SEQ ID NO: 50) |
| GUCGGGUUUAAAUAGUUUCUU | (SEQ ID NO: 21) |
| ACCCUAACCAUGUGGUAUUUU | (SEQ ID NO: 51) |
| UUUGGGAUUGGUACACCAUAG | (SEQ ID NO: 22) |
| GUACAAUUUAGCUGGACAUUU | (SEQ ID NO: 52) |
| UUCAUGUUAAAUCGACCUGUA | (SEQ ID NO: 23) |
| CUUCAAUAAACAUCAAAUAUU | (SEQ ID NO: 53) |
| CAGAAGUUAUUUGUAGUUUAU | (SEQ ID NO: 24) |
| UCAUACAUUAUUAGAUAAAUU | (SEQ ID NO: 54) |
| ACAGUAUGUAAUAAUCUAUUU | (SEQ ID NO: 25) |
| GCACAGCAACAUUAGUAAUUU | (SEQ ID NO: 55) |
| UACGUGUCGUUGUAAUCAUUA | (SEQ ID NO: 26) |
| CUCCGAUUGAAUAGUUAUAUU | (SEQ ID NO: 56) |
| UUGAGGCUAACUUAUCAAUAU | (SEQ ID NO: 27) |
| CACCUAGUUUAUAGUUAUAUU | (SEQ ID NO: 57) |
| UAGUGGAUCAAAUAUCAAUAU | (SEQ ID NO: 28) |
| CCAAUAGACACAAACUUUCUU | (SEQ ID NO: 59) |
| UCGGUUAUCUGUGUUUGAAAG | (SEQ ID NO: 30) |
| AUGAAGAAACCAUCUCACUUU | (SEQ ID NO: 60) |
| AGUACUUCUUUGGUAGAGUGA | (SEQ ID NO: 31) |
| CGCUAUAAACCAUCUCACUUU | (SEQ ID NO: 61) |
| UGGCGAUAUUUGGUAGAGUGA | (SEQ ID NO: 32) |
| ACAACCAACCCUCUGUGAUUU | (SEQ ID NO: 62) |
| UUUGUUGGUUGGGAGACACCA | (SEQ ID NO: 33) |
| ACCACCCACCCUCUGUGAUUU | (SEQ ID NO: 63) |
| AUUGGUGGGUGGGAGACACCA | (SEQ ID NO: 34) |
| CAACCAACCCUCUGUGGUUUU | (SEQ ID NO: 64) |
| UUGUUGGUUGGGAGACACCAA | (SEQ ID NO: 35) |
| CCACCCACCCUCUGUGGUUUU | (SEQ ID NO: 65) |
| UUGGUGGGUGGGAGACACCAA | (SEQ ID NO: 36) |
| AUCCCUUAACUAAACUAUAUU | (SEQ ID NO: 70) |
| AUUAGGGAAUUGAUUUGAUAU | (SEQ ID NO: 68) |
| CAGACAUUCGAUAAUAUAAUU | (SEQ ID NO: 71) |
| UAGUCUGUAAGCUAUUAUAUU | (SEQ ID NO: 69) |

It will be understood by one skilled in the art that exemplary seeds, and their complete complements, also subsume any number of shorter seeds and their complete complements, respectively, and that these are envisaged as part of the invention. For example, the 10-base seed: ACAAACUUUC (SEQ ID NO: 6) comprises further two 9-base, three 8-base, four 7-base, and five 6-base seeds, all of which could be used in the design of useful multitargeting interfering RNA.

Preferably the above multitargeting interfering RNA molecules of Formulae (I) and (II) also include at least one modified ribonucleotide or analogue, universal base, acyclic nucleotide, abasic nucleotide, non-ribonucleotide, overhang variation or a combination thereof. The multitargeting interfering RNA molecule may comprise at least one 2′-O-methyl ribosyl substitution or a locked nucleic acid ribonucleotide.

Vectors comprising a nucleotide sequence that encodes the multitargeting interfering RNA molecules of this invention are also contemplated. One type of vector is a “plasmid” which refers to a circular double stranded DNA loop into which additional DNA segments can be inserted. Preferred vectors are viral vectors. Preferred viral vectors may be selected from the group consisting of an adeno-associated virus, a retrovirus, an adenovirus, a lentivirus, and an alphavirus. The invention also relates to cells comprising the vectors of this invention.

The multitargeting interfering RNA molecules of this invention can also be short hairpin RNA molecules.

The invention further relates to pharmaceutical compositions comprising the multitargeting interfering RNA molecules of this invention and an acceptable carrier. Alternatively, the composition can include a vector comprising the RNA molecule and an acceptable carrier.

The invention further relates to methods of using the multitargeting interfering RNA molecules of this invention. In a preferred method for using the multitargeting interfering RNA molecules of this invention, the method includes inducing RNA interference in a biological system, comprising the step of introducing a multitargeting interfering RNA molecule of this invention into the biological system. More specifically, the invention relates to methods of inducing RNA interference in a biological system, comprising the steps of: (a) selecting one or more target RNA molecules; (b) designing a multitargeting interfering RNA molecule which can form stable interactions with at least two binding sequences present in distinct genetic contexts in the set of one or more target RNA molecules wherein at least one of the target RNA molecules is present in paramyxovirus RNA or coronavirus, other than SARS, RNA; (c) producing the multitargeting interfering RNA molecule; and (d) administering the multitargeting interfering RNA molecule into the biological system, whereby the multitargeting interfering RNA molecule forms stable interactions with the binding sequences present in distinct genetic contexts in the target RNA molecules, and thus induces RNA interference of the target RNA molecules. Preferably the biological system is an animal or isolated animal cell. Preferred animals include rats, mice, monkeys, and humans. The target molecules may occur in distinct genetic contexts in one virus or in more than one virus. Also preferably the target RNA molecules other than the virus target comprise RNA molecules that are involved in a disease or disorder. For example, in treating RSV infection, in addition to RSV RNA the target RNA may be host mRNA encoding proteins involved in RSV infection including proteins involved in inflammation such as IL-8, receptors to which RSV can bind such as heparan sulphate, GTP-binding proteins such as RhoA, cytoskeletal proteins such as actin or profilin and heat shock proteins such as Hsp70 (Sugrue, 2006). In regard to coronavirus infection host proteins that may be targeted in addition to the virus include cellular receptors such as angiotensin converting enzyme (ACE2), human aminopeptidase N, receptor glycoproteins and HLA class I antigens as well as proteins involved in signal transduction including MEK1/2 or ERF1/2. If targeting HPIV along with a host protein, host proteins that could be targeted for knockdown, thereby reducing the replication or survival of the virus include proteins involved in heparan sulfate synthesis such as heparan sulfate synthase, cytoskeletal proteins such as actin, sialylglycoprotein cellular receptors and protein synthesis and folding proteins such as Hsp90.

It is also contemplated within the scope of this invention that the invention further comprises a pharmaceutical composition comprising a therapeutically effective amount of one or more multitargeting interfering RNA molecules together with a pharmaceutically acceptable carrier.

In another embodiment the present invention provides the use of the multitargeting interfering RNA molecules in the preparation of a medicament for the treatment or suppression of virus infection wherein the virus is a paramyxovirus or coronavirus other than SARS.

Other aspects, features and advantages of the invention will be apparent from the following disclosure, including the detailed description of the invention and its preferred embodiments and the appended claims.

### BRIEF DESCRIPTION OF THE DRAWINGS

FIG. 1: Activity of RSV VIROMIRs and positive control siRNA (siRSVP1&2) against a GFP-RSV model in A549 cells. The data are plotted as % inhibition of the RSV-GFP virus. The 100% inhibition was set as being equal to cells not infected with virus (no GFP fluorescence), whereas 0% inhibition was set at the fluorescence of infected cells treated with mock transfection (maximum GFP fluorescence). Transfections were performed in triplicate. Error bars indicate standard deviation of the mean. A, untransfected and uninfected A549 cells; B, Mock-transfected; C, siRSVP1; D, siRSVP2; E, RS001; F, RS002; G, RS003; H, RS004; I, RS005; J, RS006; K, RS007; L, RS008; M, RS009; N, RS010; O, RS011; P, RS012; Q, RS013; R, RS016; S, RS017; T, RS018; U, RS019; V, RS020; W, RS021; X, RS022; Y, RS023; Z, RS024; AA, RS025; AB, RS026; AC, RS027; AD, RS028; AE, RS030; AF, RS031.

FIG. 2. Activity of RSV VIROMIRs and positive control siRNA (siIL-8) against IL-8 production in A549 cells. A549 cells were transfected with 40 nM duplex RNA and expression levels of secreted IL-8 measured by ELISA 72 hours post-transfection. Error bars indicate standard deviation of the mean. A, untreated; B, Mock; C, siIL-8; D, RS001; E, RS002; F, RS003; G, RS004; H, RS005; I, RS006; J, RS007; K, RS008; L, RS009; M, RS010; N, RSO11; O, RS012; P, RS013; Q, RS016; R, RS017; S, RS018; T, RS019; U, RS020; V, RS021; W, RS022; X, RS023; Y, RS024; Z, RS025; AA, RS026; AB, RS027; AC, RS028; AD, RS029; AE, RS030; AF, RS031.

FIG. 3. Effect of RSV VIROMIRs on eGFP transgene expression of A549 cells transiently transfected with the RSV-eGFP reporter construct. A549 cells in 6-well plates were co-transfected with 500 ng plasmid+300 ng duplex RNA (0 ng RNA duplex in untransfected cells) using 5 μL Lipofectamine 2000. Triplicate samples were harvested 48 hours post-transfection and analysed for GFP fluorescence by FACS. Results are shown as mean fluorescence of 10,000 cells expressed as % of control (control siRNA, siGC47). Each bar represents mean of triplicate samples. A, Plasmid plus siGC47; B, siRSVP1; C, siRSVP2; D, RS001; E, RS016.

FIG. 4. Effect of RSV VIROMIRs on eGFP transgene expression of A549 cells transiently transfected with the RSV-eGFP reporter construct. A549 cells in 6-well plates were co-transfected with 500 ng plasmid+300 ng duplex RNA (0 ng RNA duplex in untransfected cells) using 5 μL Lipofectamine 2000. Triplicate samples were harvested 48 hours post-transfection and analysed for GFP fluorescence by FACS. Results are shown as mean fluorescence of 10,000 cells expressed as % of control (control siRNA, siGC47). Each bar represents mean of triplicate samples. A, Plasmid alone; B, RS022; C, RS023; D, RS026; E, RS027.

FIG. 5. Activity of RSV VIROMIRs and positive control siRNA (siRSVP1&2) against a GFP-RSV model in A549 cells. The data are plotted as % inhibition of the RSV-GFP virus. The 100% inhibition was set as being equal to cells not infected with virus (no GFP fluorescence), whereas 0% inhibition was set at the fluorescence of infected cells treated with mock transfection (maximum GFP fluorescence). Transfections were performed in triplicate. Error bars indicate standard deviation of the mean. A, untransfected and uninfected A549 cells; B, Mock-transfected; C, siRSVP1; D, siRSVP2; E, RS014; F, RS015.

FIG. 6. Effect of RSV CODEMIRs on eGFP transgene expression of A549 cells transiently transfected with the RSV-eGFP reporter construct. A549 cells in 6-well plates were co-transfected with 300 ng, 500 ng or 750 ng PD1 plasmid±300 ng duplex RNA using 5 μL Lipofectamine 2000. Single (300 ng, 750 ng) or triplicate (500 ng) samples were harvested 48 hours post-transfection and analysed for GFP fluorescence by FACS. Results are shown as mean fluorescence of 10,000 cells expressed as % of control (control siRNA, siGC47). Each bar represents mean of triplicate samples. A, siGC47; B, siRSVP1; C, siRSVP2; D, RS014; E, RS015. Closed bars 300 ng; Striped bars 500 ng; Open bars 750 ng of PD1 plasmid, respectively

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. In this invention, certain terms are used frequently, which shall have the meanings set forth as follows. These terms may also be explained in greater detail later in the specification.

The following are abbreviations that are at times used in this specification:
bp=base pair
cDNA=complementary DNA
CODEMIR=COmputationally-DEsigned Multi-targeting Interfering RNA
HPIV=Human Parainfluenza Virus
kb=kilobase; 1000 base pairs
kDa=kilodalton; 1000 dalton
mRNA=messenger RNA
miRNA=microRNA
ncRNA=non-coding RNA
nt=nucleotide
PAGE=polyacrylamide gel electrophoresis
PCR=polymerase chain reaction
RISC=RNA Interference Silencing Complex
RNAi=RNA interference
RSV=Respiratory Syncytial Virus
SDS=sodium dodecyl sulfate
siRNA=short interfering RNA
shRNA=short hairpin RNA
SNPs=single nucleotide polymorphisms
UTR=untranslated region
VIROMIR=multitargeting interfering RNA preferentially targeted to a viral target or targets, or viral and host targets.

It must be noted that as used herein and in the appended claims, the singular forms “a,” “an,” and “the” include plural reference unless the context clearly dictates otherwise. Thus, for example, a reference to “a cell” is a reference to one or more cells and includes equivalents thereof known to those skilled in the art and so forth.

An “activity”, a “biological activity”, or a “functional activity” of a polypeptide or nucleic acid refers to an activity exerted by a polypeptide or nucleic acid molecule as determined in vivo or in vitro, according to standard techniques. Such activities can be a direct activity, such as an association with or an enzymatic activity on a second protein, or an indirect activity, such as a cellular signaling activity mediated by interaction of a protein with a second protein.

By “biological system” is meant, material, in a purified or unpurified form, from biological sources, including but not limited to human, animal, plant, insect, microbial, viral or other sources, wherein the system comprises the components required for biologic activity (e.g., inhibition of gene expression). The term “biological system” includes, for example, a cell, a virus, a microbe, an organism, an animal, or a plant.

A “cell” means an autonomous self-replicating unit that may constitute an organism (in the case of unicellular organisms) or be a sub unit of multicellular organisms in which individual cells may be specialized and/or differentiated for particular functions. A cell can be prokaryotic or eukaryotic, including bacterial cells such as E. coli, fungal cells such as yeast, bird cell, mammalian cells such as cell lines of human, bovine, porcine, monkey, sheep, apes, swine, dog, cat, and rodent origin, and insect cells such as Drosophila and silkworm derived cell lines, or plant cells. The cell can be of somatic or germ line origin, totipotent or hybrid, dividing or non-dividing. The cell can also be derived from or can comprise a gamete or embryo, a stem cell, or a fully differentiated cell. It is further understood that the term “cell” refers not only to the particular subject cell, but also to the progeny or potential progeny of such a cell. Because certain modifications can occur in succeeding generations due to either mutation or environmental influences, such progeny may not, in fact, be identical to the parent cell, but are still included within the scope of the term as used herein.

The term “complementary” or “complementarity”as used herein with respect to polynucleotides or oligonucleotides (which terms are used interchangeably herein) refers to a measure of the ability of individual sequences of such poly- or oligonucleotides to associate with each other. Two major fundamental interactions in RNA are stacking and hydrogen bonding. Both contribute to free-energy changes for associations of oligoribonucleotides. The RNA-RNA interactions include the standard Watson-Crick pairing (A opposite U, and G opposite C) and the non-Watson-Crick pairing (including but not limited to the interaction through the Hoogsteen edge and/or sugar edge).

The degree of complementarity between nucleic acid sequences has significant effects on the efficiency and strength of the association between the nucleic acid sequences. “Complementarity” between two nucleic acid sequences corresponds to free-energy changes for helix formation. Thus, determination of binding free energies for nucleic acid molecules is useful for predicting the three-dimensional structures of RNAs and for interpreting RNA-RNA associations. e.g., RNAi activity or inhibition of gene expression or formation of double stranded oligonucleotides. Such determination can be made using methods known in the art.

As the skilled artisan will appreciate, complementarity, where present, can be partial, for example where at least one or more nucleic acid bases between strands can pair according to the canonical base pairing rules. For example, the sequences 5′-CTGACAATCG-3′ (SEQ ID NO: 146), and 5′-CGAAAGTCAG-3′ (SEQ ID NO: 147) are partially complementary (also referred to herein as “incompletely complementary”) to each other. “Partial complementarity” or “partially complementary” as used herein indicates that only a percentage of the contiguous residues of a nucleic acid sequence can form Watson-Crick base pairing with the same number of contiguous residues in a second nucleic acid sequence in an anti-parallel fashion. For example, 5, 6, 7, 8, 9, or 10 nucleotides out of a total of 10 nucleotides in the first oligonucleotide forming Watson-Crick base pairing with a second nucleic acid sequence having 10 nucleotides represents 50%, 60%, 70%, 80%, 90%, and 100% complementarity respectively.

Complementarity can also be total where each and every nucleic acid base of one strand is capable of forming hydrogen bonds according to the canonical base pairing rules, with a corresponding base in another, antiparallel strand. For example, the sequences 5′-CTGACAATCG-3′ (SEQ ID NO: 146) and 5′-CGATTGTCAG-3′ (SEQ ID NO: 148) are totally complementary (also referred to herein as “completely complementary”) to each other. As used herein “complete complementarity” or “completely complementary” indicates that all the contiguous residues of a nucleic acid sequence can form Watson-Crick base pairing with the same number of contiguous residues in a second nucleic acid sequence in an anti-parallel fashion.

The skilled artisan will appreciate that where there are no bases that can adequately base pair with corresponding contiguous residues in an antiparallel strand, the two strands would be considered to have no complementarity. In certain embodiments herein, at least portions of two antiparallel strands will have no complementarity. In certain embodiments such portions may comprise even a majority of the length of the two strands.

In addition to the foregoing, the skilled artisan will appreciate that in strands of equal length that are completely complementary, all sections of those strands are completely complementary to each other. Strands which are not of equal length, i.e. present in a nucleotide duplex having one or both ends not being blunt, may be considered by those of skill in the art to be completely complementary, however there will be one or more bases in the overhanging end or ends (“overhangs”) which do not have corresponding bases in the opposing strand with which to base pair. In the case of strands that are incompletely or partially complementary, it is to be understood that there may be portions or sections of the strands wherein there are several or even many contiguous bases which are completely complementary to each other, and other portions of the incompletely complementary strands which have less than complete complementarity—i.e. those sections are only partially complementary to each other.

The percentage of complementarity between a first nucleotide sequence and a second nucleotide sequence can be evaluated by sequence identity or similarity between the first nucleotide sequence and the complement of the second nucleotide sequence. A nucleotide sequence that is X % complementary to a second nucleotide sequence is X % identical to the complement of the second nucleotide sequence. The “complement of a nucleotide sequence” is completely complementary to the nucleotide sequence, whose sequence is readily deducible from the nucleotide sequence using the rules of Watson-Crick base pairing.

“Sequence identity or similarity”, as known in the art, is the relationship between two or more polypeptide sequences or two or more polynucleotide sequences, as determined by comparing the sequences. In the art, identity also means the degree of sequence relatedness between polypeptide or polynucleotide sequences, as the case can be, as determined by the match between strings of such sequences. To determine the percent identity or similarity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same or similar amino acid residue or nucleotide as the corresponding position in the second sequence, then the molecules are identical or similar at that position. The percent identity or similarity between the two sequences is a function of the number of identical or similar positions shared by the sequences (i.e., % identity=number of identical positions/total number of positions (e.g., overlapping positions)×100). In one embodiment, the two sequences are the same length. Both identity and similarity can be readily calculated. Methods commonly employed to determine identity or similarity between sequences include, but are not limited to those disclosed in Carillo et al, (1988), SIAM J. Applied Math. 48, 1073. Preferred methods to determine identity are designed to give the largest match between the sequences tested. Methods to determine identity and similarity are codified in computer programs, non-limiting examples of which are the NBLAST and XBLAST programs, Gapped BLAST, PSI-Blast, the FASTA method, the ALIGN program, the GCG GAP program, and the BestFit program in the GCG software package.

Nucleotide sequences that share a substantial degree of complementarity will form a stable interaction with each other. As used herein, the term “stable interaction” with respect to two nucleotide sequences indicates that the two nucleotide sequences have the natural tendency to interact with each other to form a double stranded molecule. Two nucleotide sequences can form a stable interaction with each other within a wide range of sequence complementarity. In general, the higher the complementarity the stronger or the more stable the interaction. Different strengths of interactions may be required for different processes. For example, the strength of interaction for the purpose of forming a stable nucleotide sequence duplex in vitro may be different from that for the purpose of forming a stable interaction between an interfering RNA and a binding sequence in vivo. The strength of interaction can be readily determined experimentally or predicted with appropriate software by a person skilled in the art.

Hybridization can be used to test whether two polynucleotides are substantially complementary to each other and to measure how stable the interaction is. Polynucleotides that share a sufficient degree of complementarity will hybridize to each other under various hybridization conditions. In one embodiment, polynucleotides that share a high degree of complementarity thus form a strong stable interaction and will hybridize to each other under stringent hybridization conditions. “Stringent hybridization conditions” has the meaning known in the art, as described in Sambrook et al., Molecular Cloning: A Laboratory Manual, Second Edition, Cold Spring Harbor Laboratory, Cold Spring Harbor, N.Y., (1989). An exemplary stringent hybridization condition comprises hybridization in 6× sodium chloride/sodium citrate (SSC) at about 45° C., followed by one or more washes in 0.2×SSC and 0.1% SDS at 50-65° C.

As used herein the term “mismatch” refers to a nucleotide of either strand of two interacting strands having no corresponding nucleotide on the corresponding strand or a nucleotide of either strand of two interacting strands having a corresponding nucleotide on the corresponding strand that is non-complementary.

As used herein, a “match” refers to a complementary pairing of nucleotides.

As used herein, the term “expression system” refers to any in vivo or in vitro system that can be used to evaluate the expression of a target RNA molecule and/or the RNAi activity of a multitargeting RNA molecule of the invention. In particular embodiments, the “expression system” comprises one or more target RNA molecules, a multitargeting interfering RNA molecule targeting the one or more target RNA molecules, and a cell or any type of in vitro expression system known to a person skilled in the art that allows expression of the target RNA molecules and RNAi.

As used herein, the term “RNA” includes any molecule comprising at least one ribonucleotide residue, including those possessing one or more natural nucleotides of the following bases: adenine, cytosine, guanine, and uracil; abbreviated A, C, G, and U, respectively, modified ribonucleotides or analogues, universal base, acyclic nucleotide, abasic nucleotide, non-ribonucleotides, or any combination thereof. “Ribonucleotide” means a nucleotide with a hydroxyl group at the T position of a p-D-ribo-furanose moiety.

Modified ribonucleotides include, for example 2′deoxy, 2′deoxy-2′-fluoro, 2′O-methyl, 2′O-methoxyethyl, 4′thio or locked nucleic acid (LNA) ribonucleotides. Also contemplated herein is the use of various types of ribonucleotide analogues, and RNA with internucleotide linkage (backbone) modifications. Modified internucleotide linkages include for example, phosphorothioate-modified, and even inverted linkages (i.e. 3′-3′ or 5′-5′). Preferred ribonucleotide analogues include sugar-modified, and nucleobase-modified ribonucleotides, as well as combinations thereof. In preferred sugar-modified ribonucleotides the 2′-OH-group is replaced by a substituent selected from H, OR, R, halo, SH, SR, NH₂, NHR, NR₂ or ON, wherein R is C1-C6 alkyl, alkenyl or alkynyl and halo is F, Cl, Br, or I. In preferred backbone-modified ribonucleotides the phosphoester group connecting to adjacent ribonucleotides is replaced by a modified group, e.g. a phosphorothioate group. Any or all of the above modifications may be combined. In addition, the 5′termini can be OH, phosphate, diphosphate or triphosphate. Nucleobase-modified ribonucleotides, i.e. ribonucleotides wherein the naturally-occurring nucleobase is replaced with a non-naturally occurring nucleobase instead, for example, uridines or cytidines modified at the S-position (e.g. 5-(2-amino)propyl uridine, and 5-bromo uridine); adenosines and guanosines modified at the 8-position (e.g. 8-bromo guanosine); deaza nucleotides (e.g. 7-deaza-adenosine); O- and N-alkylated nucleotides (e.g. N6-methyl adenosine) are also contemplated for use herein.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example where any of the ribose carbons (C1, C2, C3, C4, or C5), are independently or in combination absent from the nucleotide.

As used herein with respect to the listing of RNA sequences, the bases thymidine (“T”) and uridine (“U”) are frequently interchangeable depending on the source of the sequence information (DNA or RNA). Therefore, in disclosure of target sequences, seed sequences, candidate seeds, consensus target sequences, target RNA binding sites, and the like, the base “T” is fully interchangeable with the base “U”. However, with respect to specific disclosures of the interfering RNA molecules of the invention, it is to be understood that for such sequences the use of the base “U” cannot be generally substituted with “T” in a functional manner. It is however known in the art that certain occurrences of the base “U” in RNA molecules can be substituted with “T” without substantially deleterious effect on functionality. For example, the substitution of T for U in overhangs, such as UU overhangs at the 3′ end is known to be silent, or at a minimum, acceptable, and thus is permissible in the interfering RNA sequences provided herein. Thus, it is contemplated that the skilled artisan will appreciate how to vary even the specific interfering RNA sequences disclosed herein to arrive at other structurally-related and functionally-equivalent structures that are within the scope of the instant invention and the appended claims.

The “target RNA molecule” can be a RNA molecule that is endogenous to a biological system, or a RNA molecule that is exogenous to the biological system, such as a RNA molecule of a pathogen, for example a virus, which is present in a cell after infection thereof. A cell containing the target RNA can be derived from or contained in any organism, for example a plant, animal, protozoan, virus, bacterium, or fungus. Non-limiting examples of plants include monocots, dicots, or gymnosperms. Non-limiting examples of animals include vertebrates or invertebrates. Non-limiting examples of fungi include molds or yeasts.

A “target RNA molecule” or a “pre-selected target RNA molecule” as used herein refers to any RNA molecule whose expression or activity is desired to be modulated, for example decreased, by an interfering RNA molecule of the invention in an expression system. A “target RNA molecule” can be a messenger RNA (mRNA) molecule that encodes a polypeptide of interest. A messenger RNA molecule typically includes a coding region and non-coding regions preceding (“5′UTR”) and following (“3′UTR”) the coding region. A “target RNA molecule” can also be a non-coding RNA (ncRNA), such as small temporal RNA (stRNA), micro RNA (miRNA), pre-microRNA, pri-microRNA, small nuclear RNA (snRNA), short interfering RNA (siRNA), small nucleolar RNA (snoRNA), ribosomal RNA (rRNA), transfer RNA (tRNA) and precursor RNAs thereof. Such non-coding RNAs can also serve as target RNA molecules because ncRNA is involved in functional or regulatory cellular processes. Aberrant ncRNA activity leading to disease can therefore be modulated by multitargeting interfering RNA molecules of the invention. The target RNA can further be the genome of a virus, for example a RNA virus, or a replicative intermediate of any virus at any stage, as well as any combination of these.

A “target RNA molecule” as used herein may include any variants or polymorphism of a desired RNA molecule. Most genes are polymorphic in that a low but nevertheless significant rate of sequence variability occurs in a gene among individuals of the same species. Thus, a RNA molecule may correlate with multiple sequence entries, each of which represents a variant or a polymorphism of the RNA molecule. In designing any gene suppression tool there is the risk that the selected binding sequence(s) used in the computer-based design may contain relatively infrequent alleles. As a result, the active sequence designed might be expected to provide the required benefit in only a proportion of individuals. The frequency, nature and position of most variants (often referred to as single nucleotide polymorphisms (SNPs)) are easily accessible to those trained in the art. In this respect, sequences within a target molecule that are known to be highly polymorphic can be avoided in the selection of binding sequences during the bioinformatic screen. Alternatively, a limitless number of sequences available for any particular target may be used in the design stages of an interfering RNA of the invention to make sure that the targeted binding sequence is present in the majority of allelic variants, with the exception of the situation in which targeting of the allelic variant is desired (that is, when the allelic variant itself is implicated in the disease of interest).

A “target RNA molecule” comprises at least one targeted binding sequence that is sufficiently complementary to the guide sequence of an interfering RNA molecule of the invention to allow a stable interaction of the binding sequence with the guide sequence. The targeted binding sequence can be refined to include any part of the transcript sequence (eg. 5′UTR, ORF, 3′UTR) based on the desired effect. For example, translational repression is a frequent mechanism operating in the 3′UTR (eg. as for microRNA). Thus, the targeted binding sequence can include sequences in the 3′ UTR for effective translational repression.

The “targeted binding sequence”, “binding sequence”, or “target sequence” shall all mean a portion of a target RNA molecule sequence comprising a seed sequence and the sequence flanking either one or both ends of the seed, said binding sequence is predicted to form a stable interaction with the guide strand of a multitargeting interfering RNA of the invention based on the complementarity between the guide strand and the binding sequence.

As used herein, the term “non-target transcriptome” or “non-targeted transcriptome” indicates the transcriptome aside from the targeted RNA molecules. For example, when a multitargeting interfering RNA is designed to target a viral RNA, the non-targeted transcriptome is that of the host. When a multitargeting interfering RNA is designed to target a given RNA in a biological system, the non-targeted transcriptome is the transcriptome of the biological system aside from the targeted given RNA.

As used herein the term “seed” or “seed sequence” or “seed region sequence” refers to a sequence of at least about 6 contiguous nucleotides present in a target RNA that is completely complementary to a portion of the guide strand of an interfering RNA.

Although 6 or more contiguous bases are preferred, the expression “about 6” refers to the fact that windows of at least 5 or more contiguous bases can provide useful candidates in some cases and can ultimately lead to the design of useful interfering RNAs. Thus, all such seed sequences are contemplated within the scope of the instant invention.

“Conservation or conserved” indicates the extent to which a specific sequence, such as the seed sequence, is found to be represented in a group of related target sequences, regardless of the genetic context of the specific sequence.

“Genetic context” refers to the flanking sequences that surround a specific identified sequence and that are sufficiently long to enable one of average skill in the art to determine its position within a genome or RNA molecule relative to sequence annotations or other markers in common use.

As used herein, the term “interfering RNA” is used to indicate single or double stranded RNA molecules that modulate the presence, processing, transcription, translation, or half-life of a target RNA molecule, for example by mediating RNA interference (“RNAi”), in a sequence-specific manner. As used herein, the term “RNA interference” or “RNAi” is meant to be equivalent to other terms used to describe sequence specific RNA interference, such as post-transcriptional gene silencing, translational inhibition, or epigenetics. This includes, for example, RISC-mediated degradation or translational repression, as well as transcriptional silencing, altered RNA editing, competition for binding to regulatory proteins, and alterations of mRNA splicing. It also encompasses degradation and/or inactivation of the target RNA by other processes known in the art, including but not limited to nonsense-mediated decay, and translocation to P bodies. Thus, the interfering RNAs provided herein (e.g. CODEMIRs and VIROMIRs) may exert their functional effect via any of the foregoing mechanisms alone, or in combination with one or more other means of RNA modulation known in the art. The interfering RNAs provided herein can be used to manipulate or alter the genotype or phenotype of an organism or cell, by intervening in cellular processes such as genetic imprinting, transcription, translation, or nucleic acid processing (e.g., transamination, methylation, etc.).

The term “interfering RNA” is meant to be equivalent to other terms used to describe nucleic acid molecules that are capable of mediating sequence specific RNAi, for example short interfering RNA (siRNA), double-stranded RNA (dsRNA), microRNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

The “interfering RNA” can be, for example, a double-stranded polynucleotide molecule comprising self-complementary sense and antisense strands. The “sense” also named “passenger” strand is required for presentation of the “antisense” also named “guide”, “guiding”, or “target-complementary” strand to the RISC. The guide strand is retained in the active RISC complex and guides the RISC to the target nucleotide sequence by means of complementary base-pairing, which in turn results in RNAi. The relative thermodynamic characteristics of the 5′ termini of the two strands of a double-stranded interfering RNA determine which strand will serve the function of a passenger or a guide strand during RNAi. Indeed, the asymmetric RISC formation can be defined by the relative thermodynamic strength of the first four nucleotide-pairs of the 5′ termini of an interfering RNA calculated by the nearest-neighbor methods. Thus, in designing an interfering RNA of the invention, the guide strand can be pre-determined by the 5′ termini thermodynamic characteristics.

In an interfering RNA of the invention, the guide strand can have a sequence completely complementary to one or more but not all binding sequences present in the one or more target RNA molecules. It can also be partially complementary to a binding sequence present in a target RNA molecule, so long as the complementarity is sufficient for the formation of a stable interaction between the guide strand and the binding sequence in the target molecule. The “passenger strand” can be completely or partially complementary to the guide strand, so long as the complementarity is sufficient for the formation of a stable interaction between the guide strand and the passenger strand. Thus, the passenger strand can be completely or partially identical to the binding sequence on a target molecule. Both the passenger strand and the guide strand can be modified and refined to enhance some aspect of the function of the interfering RNA molecule of the invention. For example, various pharmacophores, dyes, markers, ligands, conjugates, antibodies, antigens, polymers, peptides and other molecules can be conveniently linked to the molecules of the invention. The interfering RNA can further comprise a terminal phosphate group, such as a 5′-phosphate or 5′,3′-diphosphate. These may be of use to improve cell uptake, stability, tissue targeting or any combination thereof.

The “interfering RNA” can be assembled from two separate oligonucleotides. It will be appreciated that in the alternative embodiments according to Formula (II), the strands can be adjusted to achieve approximately equal loading of each into the RISC. In this embodiment both strands are designed to act as guide strands. The “interfering RNA” can also be assembled from a single oligonucleotide, where the self-complementary regions of the interfering RNA are linked by means of a nucleic acid based or non-nucleic acid-based linker(s). The “interfering RNA” can be a polynucleotide with a duplex, asymmetric duplex, hairpin or asymmetric hairpin secondary structure, having self-complementary sense and antisense regions. The “interfering RNA” can also be a single-stranded polynucleotide having one or more loop structures and a stem comprising self-complementary regions (e.g. short hairpin RNA, shRNA), wherein the polynucleotide can be processed either in vivo or in vitro to generate one or more double stranded interfering RNA molecules capable of mediating RNA interference. The cleavage of the self-paired region or regions of the single strand RNA to generate double-stranded RNA can occur in vitro or in vivo, both of which are contemplated for use herein.

In embodiments, for example, of Formula (I), the “interfering RNA” can also be a single stranded polynucleotide having nucleotide sequence complementary to a nucleotide sequence in a target nucleic acid molecule or a portion thereof (i.e., the guide strand), for example, where such interfering RNA molecule does not require the presence within the molecule of nucleotide sequence corresponding to the target nucleic acid sequence or a portion thereof (i.e., the passenger strand).

As used herein, the term “interfering RNA” need not be limited to those molecules containing only RNA, but further encompasses those possessing one or more modified ribonucleotides and non-nucleotides, such as those described supra.

The term “interfering RNA” includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the multitargeting interfering RNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

The interfering RNA of the invention, also termed “multitargeting interfering RNA” is an interfering RNA having a guide strand that can form stable interactions with at least two binding sites present in distinct genetic contexts in one or more target RNA molecules. Examples of the multitargeting interfering RNA include CODEMIRs, COmputationally-DEsigned Multi-targeting Interfering RNAs, and VIROMIRs, where these multitargeting interfering RNA molecules are preferentially targeted to viral targets (either single or multiple) or a viral and host target. The term CODEMIR may in some aspects encompass a VIROMIR.

“Sequence” means the linear order in which monomers occur in a polymer, for example, the order of amino acids in a polypeptide or the order of nucleotides in a polynucleotide.

A “subject” as used herein, refers to an organism to which the nucleic acid molecules of the invention can be administered. A subject can be an animal or a plant, preferably a mammal, most preferably a human, who has been the object of treatment, observation or experiment, or any cell thereof.

The term “therapeutically effective amount” as used herein, means that amount of active compound or pharmaceutical agent that elicits the biological or medicinal response in a subject that is being sought by a researcher, veterinarian, medical doctor or other clinician, which includes preventing, ameliorating or alleviating the symptoms of the disease or disorder being treated. Methods are known in the art for determining therapeutically effective doses for the instant pharmaceutical composition.

A “vector” refers to a nucleic acid molecule capable of transporting another nucleic acid to which it has been linked. One type of vector is a “plasmid”, which refers to a circular double stranded DNA loop into which additional DNA segments can be inserted. Another type of vector is a viral vector, wherein additional DNA segments can be inserted. Certain vectors are capable of autonomous replication in a host cell into which they are introduced (e. g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked.

As used herein, “modulate (or modulation of) the expression of an RNA molecule” means any RNA interference mediated regulation of the level and/or biological activity of the RNA molecule. It includes any RNAi-related transcriptional or post-transcriptional gene silencing, such as by cleaving, destabilizing the target RNA molecule or preventing RNA translation. In one embodiment, the term “modulate” can mean “inhibit,” but the use of the word “modulate” is not limited to this definition. The modulation of the target RNA molecule is determined in a suitable expression system, for example in vivo, in one or more suitable cells, or in an acellular or in vitro expression system such as are known in the art. Routine methods for measuring parameters of the transcription, translation, or other aspects of expression relating to RNA molecules are known in the art, and any such measurements are suitable for use herein.

By “inhibit”, “down-regulate”, “reduce”, or “decrease” as with respect to a target RNA or its expression it is meant that the expression of the gene or level and/or biological activity of target RNA molecules is reduced below that observed in the absence of the nucleic acid molecules (e.g., multitargeting interfering RNA) of the invention. In one embodiment, inhibition, down-regulation or reduction with a multitargeting interfering RNA molecule is greater than that observed in the presence of an inactive or attenuated molecule. In another embodiment, inhibition, down-regulation, or reduction with a multitargeting interfering RNA molecule is greater than that observed in the presence of, for example, multitargeting interfering RNA molecule with scrambled sequence or with mismatches. In another embodiment, inhibition, down-regulation, or reduction of gene expression with a nucleic acid molecule of the instant invention is greater in the presence of the nucleic acid molecule than in its absence.

“Inhibit”, “down-regulate”, “reduce”, or “decrease” as with respect to a target RNA or its expression encompasses, for example, reduction of the amount or rate of transcription or translation of a target RNA, reduction of the amount or rate of activity of the target RNA, reduction in the rate of viral replication, and/or a combination of the foregoing in a selected expression system. The skilled artisan will appreciate that a decrease in the total amount of transcription, the rate of transcription, the total amount of translation, the rate of translation, or the rate of viral replication, or even the activity of an encoded gene product are indicative of such a decrease. The “activity” of an RNA refers to any detectable effect the RNA may have in a cell or expression system, including for example, any effect on transcription, such as enhancing or suppressing transcription of itself or another RNA molecule. The measurement of a “decrease” in expression or the determination of the activity of a given RNA can be performed in vitro or in vivo, in any system known or developed for such purposes, or adaptable thereto. Preferably the measurement of a “decrease” in expression by a particular interfering RNA is made relative to a control, for example, in which no interfering RNA is used. In some comparative embodiments such measurement is made relative to a control in which some other interfering RNA or combination of interfering RNAs is used. Most preferably a change, such as the decrease is statistically significant based on a generally accepted test of statistical significance. However, because of the large number of possible measures and the need for the ability to rapidly screen candidate interfering RNAs, it is contemplated herein that a given RNA need only show an arithmetic decrease in one such in vitro or in vivo assay to be considered to show a “decrease in expression” as used herein.

More particularly, the biological modulating activity of the multitargeting interfering RNA is not limited to, or necessarily reliant on, degradation or translational repression by conventional RISC protein complexes involved in siRNA and microRNA gene-silencing, respectively. Indeed, short double-stranded and single-stranded RNA have been shown to have other possible sequence-specific roles via alternative mechanisms. For example, short double-stranded RNA (dsRNA) species may act as modulatory effectors of differentiation/cell activity, possibly through binding to regulatory proteins. Alternatively, dsRNA may lead to the degradation of mRNA through the involvement of AU-rich element (ARE)-binding proteins. Further, dsRNA may also induce epigenetic transcriptional silencing. Processing of mRNA can also be altered through A to I editing and modified splicing.

As used herein, “palindrome” or “palindromic sequence” means a nucleic acid sequence that is completely complementary to a second nucleotide sequence that is identical to the nucleic acid sequence, e.g., UGGCCA. The term also includes a nucleic acid molecule comprising two nucleotide sequences that are palindromic sequences.

“Phenotypic change” as used herein refers to any detectable change to a cell or an organism that occurs in response to contact or treatment with a nucleic acid molecule of the invention. Such detectable changes include, but are not limited to, changes in shape, size, proliferation, replication, motility, protein expression or RNA expression or other biological, physical or chemical changes as can be assayed by methods known in the art. The detectable change can also include expression of reporter genes/molecules such as Green Fluorescent Protein (GFP) or various tags that are used to identify an expressed protein or any other cellular component that can be assayed.

The present invention provides a multitargeting interfering RNA molecule comprising a guide strand, or two guide strands, that form stable interactions with at least two binding sequences present in distinct genetic contexts in one or more pre-selected target RNA molecules wherein at least one of the binding sequences is present in paramyxovirus RNA or coronavirus, other than SARS, RNA.

In one general aspect, the present invention provides a multitargeting interfering RNA molecule comprising a guide strand of the Formula (I):

| | |
|---|---|
| 5′-p-XSY-3′ | (Formula I) |

In Formula (I), p consists of a terminal phosphate group that can be present at, or absent from, the 5′-end of the guide strand. Any terminal phosphate group known to a person skilled in the art can be used. Such phosphate groups include, but are not limited to, monophosphate, diphosphate, triphosphate, cyclic phosphate or to a chemical derivative of phosphate such as a phosphate ester linkage.

In Formula (I), S consists of a first nucleotide sequence of a length of about 5 to about 20 nucleotides that is at least partially, preferably completely, complementary to a first portion of each of at least two binding sequences present in distinct genetic contexts in one or more pre-selected target RNA molecules. In particular embodiments, S has a length of about 6 to about 15 nucleotides, such as a length of 6, 7, 8, 9, 10, 11, 12, 13, 14, or 15 nucleotides that are at least partially, preferably completely, complementary to the first portion of the at least two binding sequences. In one embodiment, S is completely complementary to a seed sequence of each of one, two, three, four, five, or more distinct binding sequences present in distinct genetic contexts in one or more pre-selected target RNA molecules. The skilled artisan will appreciate that the at least two distinct binding sequences may be on the same target RNA molecule, or they can be on different RNA molecules.

In certain embodiments, S is partially complementary to a first portion of at least two distinct binding sequences present in distinct genetic contexts in one or more pre-selected target RNA molecules, such as 6 of 7, 7 of 8, 8 of 9, 9 of 10, 10 of 11, 11 of 12, 12 of 13, 13 of 14, 14 of 15, or 15 of 16 consecutive nucleotides of S are completely complementary to the first portion of at least two target RNA binding sequences. In other embodiments, S and the first portion of the distinct binding sequences have lesser overall complementarity such as 10 of 12, 11 of 13, 12 of 14, 13 of 15, or 14 of 16 nucleotides of complete complementarity.

In Formula (I), X is absent or consists of a second nucleotide sequence. In particular embodiments, X consists of one or two nucleotides. In some embodiments it may consist of thre or more nucleotides.

In Formula (I), Y is absent or consists of a third nucleotide sequence, provided that X and Y are not absent simultaneously. Y has complementarity that ranges from complete to nonexistent with respect to a second portion of each of the at least two distinct binding sequences, where the second portion is adjacent to and connected with the 5′-end of the first portion of the binding sequences. In one embodiment, Y is at least partially complementary to a second portion of at least one binding sequence, thus allowing the guide strand to have improved interaction with the at least one binding sequence. Preferably, Y provides optimal or desired binding to each of the second portions of the distinct binding sequences by comprising a consensus-like sequence to which these second portions can bind. This aspect of having a region of less than complete complementarity in the guide strand is particularly useful in certain embodiments, for example, by providing an area of some consensus between distinct binding sequences.

In particular embodiments of the invention, by combining in the guide strand, S with complete complementarity to a seed portion of each binding sequence, and Y, that is incompletely complementary to a second portion of each binding sequence, the overall nucleotide sequence of XSY is such that it is at least partially complementary to each of the distinct binding sequences to allow a stable interaction with each of the binding sequences, thus providing multitargeting interfering RNA of any target molecules comprising the binding sequences. In some embodiments XSY may be fully complementary to at least one of the distinct binding sequences. In other embodiments, XSY is partially complementary to the distinct binding sequences.

The multitargeting interfering RNA can comprise both a guide strand of formula (I) described supra and a passenger strand that is at least partially complementary to the guide strand to allow formation of stable duplexes between the passenger strand and the guide strand. The passenger strand and the guide strand can be completely complementary to each other. The passenger strand and the guide strand can have the same or different length. In an embodiment of the present invention, each strand of a multitargeting interfering RNA molecule of the invention is independently about 17 to about 25 nucleotides in length, in specific embodiments about 17, 18, 19, 20, 21, 22, 23, 24, and 25 nucleotides in length. Using shorter length interfering RNA molecules without the need for the generation of multiple active sequences through processing of RNA by enzymes such as Dicer and RNaseIII, provides advantages, for example, in reduction of cost, manufacturing, and chance of off-target effects.

In an alternative embodiment this invention relates to a multitargeting interfering RNA molecule comprising Formula (II):

5′-p-A B C-3′
3′-A′B′C′-p-5′

wherein p consists of a terminal phosphate group that is independently present or absent; wherein B consists of a first nucleotide sequence of a length of about 5 to about 20 nucleotides that is partially, preferably completely, complementary to a first portion of a first binding sequence, and if consists of a second nucleotide sequence of a length of about 5 to about 20 nucleotides that is partially, preferably completely, complementary to a first portion of a second binding sequence, wherein said first and second binding sequences are present in distinct genetic contexts in at least one pre-selected target RNA molecule, and wherein B and if are at least substantially complementary to each other but are not palindromic; and further wherein A, A′, C, or C′, is independently absent or consists of a nucleotide sequence; wherein ABC is at least partially complementary to the first binding sequence to allow stable interaction therewith; and wherein C′B′A′ is at least partially complementary to the second binding sequence to allow stable interaction therewith and is at least partially complementary to ABC to form a stable duplex therewith and wherein at least one of the first or second binding sequences is present in paramyxovirus RNA or coronavirus, other than SARS, RNA.

The terminal phosphate group, p, can be present or absent from the 5′-end of either strand. Any terminal phosphate group known to a person skilled in the art can be used. Such phosphate group includes, but is not limited to, monophosphate, diphosphate, triphosphate, cyclic phosphate or to a chemical derivative of phosphate such as a phosphate ester linkage.

In particular embodiments, B and B′ each has a length of, for example, 5, 6, 7, 8, 9, 10, 11, 12, 13, 14, 15, 16, 17, 18, 19, or 20 nucleotides that are at least partially, preferably completely, complementary to the first portion of the at least two binding sequences. In one embodiment, B is completely complementary to a sequence present in one or more pre-selected target RNA molecules. In another embodiment, B′ is completely complementary to a sequence present in one or more pre-selected target RNA molecules. In particular embodiments, B and if are completely complementary to each other.

In certain embodiments, B is partially complementary to a first portion of a binding sequence present in one or more pre-selected target RNA molecules, such as 6 of 7, 7 of 8, 8 of 9, 9 of 10, 10 of 11, 11 of 12, 12 of 13, 13 of 14, 14 of 15, or 15 of 16 consecutive nucleotides of B are completely complementary to the first portion of at least one target RNA binding sequence. In other embodiments, B and the first portion of the distinct binding sequences have lesser overall complementarity such as 10 of 12, 11 of 13, 12 of 14, 13 of 15, or 14 of 16 nucleotides of complete complementarity. Similarly, in certain embodiments, B′ is partially complementary to a first portion of a second binding site.

The remaining sequence of the two strands of the multitargeting interfering RNA (A, A′, C and C′) in Formula (II) is independently absent or consists of a nucleotide sequence. In particular embodiments, they are developed so as to generate further binding to the target RNA sites. In one embodiment, the sequences of A and C′ are at least partially complementary to the second portions of the first and second target RNA binding sequences, respectively. In one embodiment, the sequences A′ and C are completely complementary to A and C′, respectively, such that ABC and C′B′A′ are completely complementary. In an additional embodiment, A′ and C are incompletely complementary with A and C′, respectively such that ABC and C′B′A′ are incompletely complementary. This may be required, for example, in situations in which the loading bias of the interfering RNA duplex needs to be altered through the use of mismatches in the extremity with the higher hybridization energy.

In a further embodiment, the sequences A and C′ are designed so as to maximize binding of AB and C′B′ to the first and second portions of a plurality of target RNA binding sites. In this situation, the plurality of target sequences (e.g. viral isolates) can be examined in order to generate a number of identity consensus sequences corresponding to the second portion of the plurality of target RNA sequences. These identity consensus sequences can be generated by hand by examining the alignments of the target RNA sequences. Alternatively, all possible base sequences or a subset of putative AB and C′B′ sequences can be generated by computer algorithm. Each putative AB and C′B′ sequence is then hybridized in silico using RNAhybrid or a similar program known to one skilled in the art. Those putative sequences that are predicted to best bind the corresponding first and second portions of the target RNA binding sites are then prioritized for the next design phase, which includes filtering out putative sequences that have unfavorable characteristics such as more than 4 contiguous C or G bases.

In a preferred embodiment, the sequences of C and A′ are then designed such that they are at least partially complementary to C′ and A, respectively. Overhangs, if required may simply be the addition to A′ and C of, for example, UU, dTdT or any other base or modified base. In one embodiment, the bases of the overhangs are selected so as to further increase the predicted binding of ABC and C′B′A′ to their respective RNA targets. Overhangs may be 1, 2, 3, 4 or 5 bases as required.

In an interfering RNA of the invention, a preferred embodiment is one in which the two strands of the duplex independently have either partial or complete complementarity to their corresponding at least one target sequence and the two strands are completely complementary to one another, excepting the overhangs when present. Another embodiment of the invention is one in which each of the two strands of the duplex independently have either partial or complete complementarity to their corresponding at least one target sequence and the two strands are incompletely complementary to one another. Both strands can be modified and refined to enhance some aspect of the function of the interfering RNA molecule of the invention. For example, various pharmacophores, dyes, markers, ligands, conjugates, antibodies, antigens, polymers, peptides and other molecules can be conveniently linked to the molecules of the invention. The interfering RNA can further comprise one or more 5′ terminal phosphate group, such as a 5′-phosphate or 5′,3′-diphosphate. These may be of use to improve cell uptake, stability, tissue targeting or any combination thereof.

In another embodiment, A consists of a nucleotide sequence that is at least partially complementary to a second portion of the first binding sequence, said second portion is adjacent to and connected with the 3′-end of said first portion of the first binding sequence, and wherein A′ is substantially complementary to A. In a particular embodiment, A and A′ are completely complementary to each other. In another particular embodiment, A is completely complementary to the second portion of the first binding sequence.

In yet another embodiment, C′ consists of a nucleotide sequence that is at least partially complementary to a second portion of the second binding sequence, said second portion is adjacent to and connected with the 3′-end of said first portion of the second binding sequence, and wherein C is substantially complementary to C′. In a particular embodiment, C and C′ are completely complementary to each other. In another particular embodiment, C′ is completely complementary to the second portion of the second binding sequence.

In Formula (II), ABC is at least partially complementary to the first binding sequence to allow stable interaction of ABC with the first binding sequence, and C′B′A′ is at least partially complementary to the second binding sequence to allow stable interaction with the second binding sequence, and ABC and C′B′A′ are at least partially complementary to each other to allow formation of a stable duplex. In a particular embodiment, ABC is completely complementary to the first binding sequence. In another embodiment, C′B′A′ is completely complementary to the second binding sequence. In yet another embodiment, ABC and C′B′A′ are completely complementary to each other.

In an embodiment of the present invention, each strand of a multitargeting interfering RNA molecule of Formulae (I) or (II) is independently about 17 to about 25 nucleotides in length, in specific embodiments about 17, 18, 19, 20, 21, 22, 23, 24, and 25 nucleotides in length. Using shorter length interfering RNA molecules without the need for the generation of multiple active sequences through processing of RNA by enzymes such as Dicer and RNaseIII, provides advantages, for example, in reduction of cost, manufacturing, and chance of off-target effects.

The interaction between the passenger strand and the guide strand can be adjusted to improve loading of the guide strand into the cellular RISC complex, or to otherwise improve the functional aspects of the multitargeting interfering RNA. The skilled artisan will appreciate that there are routine methods for altering the strength and other properties of the base paired strands through the addition, deletion, or substitution of one or more bases in either strand of the synthetic duplex. In particular as one example, these strategies can be applied to the design of the extremities of the duplex to ensure that the predicted thermodynamics of the duplex are conducive to the loading of the desired strand. These strategies are well known to persons skilled in the art.

It will be appreciated that in the alternative embodiments of this invention according to Formula (II), the strands can be adjusted to achieve approximately equal loading of each into the RISC. In this embodiment, both strands are designed to act as guide strands.

It is also contemplated herein that a substantially double-stranded RNA molecule comprises a single-stranded RNA molecule with, for example, a hairpin loop or similar secondary structure that allows the molecule to self-pair to form at least a region of double-stranded nucleic acid comprising the guide strand of Formula (I) or at least a region of double-stranded nucleic acid of Formula (II).

It will be appreciated by one skilled in the art that when a dsRNA of formulae I or II is considered, the loading bias may be manipulated through the use of wobble base pairing or mismatches such that the binding affinity at one end of the dsRNA duplex is modulated and loading improved. In the case of a molecule designed according to formula II, this approach may be used to ensure both strands are loaded.

The skilled artisan will appreciate that the double-stranded RNA molecules provide certain advantages for use in therapeutic applications. Although blunt-ended molecules are disclosed herein for certain embodiments, in various other embodiments, overhangs, for example of 1-5 nucleotides, are present at either or both termini. In some embodiments, the overhangs are 2 or 3 bases in length. Presently preferred overhangs include 3′-UU overhangs in certain embodiments. Other overhangs exemplified for use herein include, but are not limited to, 3′-AA, 3′-CA, 3′-AU, 3′-UC, 3′-CU, 3′-UG, 3′-CC, 3′-UA, 3′-U, and 3′-A. Still other either 5′-, or more preferably 3′-, overhangs of various lengths and compositions are contemplated for use herein on the RNA molecules provided.

In an embodiment of the invention the multitargeting interfering RNA targets virus RNA and host RNA which encodes a protein involved in the infection process. Examples of host proteins involved in RSV infection include proteins involved in inflammation such as IL-8, receptors to which RSV can bind such as heparan sulphate, GTP-binding proteins such as RhoA, cytoskeletal proteins such as actin or profilin and heat shock proteins such as Hsp70 (Sugrue, 2006).

If targeting HPIV along with a host protein, host proteins that could be targeted for knockdown, thereby reducing the replication or survival of the virus include proteins involved in heparan sulfate synthesis such as heparan sulfate synthase, cytoskeletal proteins such as actin, sialylglycoprotein cellular receptors and protein synthesis and folding proteins such as Hsp90.

Host proteins that could be targeted for knockdown in treating or suppressing coronavirus infection, and in doing so, reduce the replication or survival of the virus include cellular receptors such as angiotensin converting enzyme (ACE2), human aminopeptidase N, receptor glycoproteins and HLA class I antigens as well as proteins involved in signal transduction including MEK1/2 or ERF1/2.

In one embodiment, at least one of the binding sequences is in the 3′ UTR of an mRNA. In embodiments featuring multitargeting of different RNA molecules, preferably the target RNAs are not solely splice variants of a single gene, nor solely isoforms of each other. In other embodiments where it is vital or preferred to modulate some or all such splice variants or isoforms, the multiple targets may encompass such sequences.

The inclusion of one target or more targets does not preclude the use of, or intention for, a particular interfering RNA to target another selected target. Such targeting of any additional RNA target molecules may result in less, equal, or greater effect in an expression system. Notwithstanding the foregoing, the multitargeting interfering RNAs of the instant invention are preferably screened for off-target effects, especially those that are likely. For example, reviewing the potential binding to the entire transcriptome, or as much as of it as is known at the time provides a useful approach to such screening. For example, where a genome has been completely sequenced, the skilled artisan will appreciate that the entire transcriptome can be conveniently screened for likely off-target effects. In cases for which local delivery of multitargeting interfering RNA is anticipated, specialized tissue-specific transcriptomes (eg lung for inhaled therapeutics) may be more relevant because non-target transcripts that are identified through bioinformatic approaches from the complete transcriptome may actually not be present in the tissue into which the multitargeting interfering RNA is applied.

In one embodiment, the multitargeting interfering RNA of the invention forms stable interactions with at least two targeted binding sequences present in distinct genetic contexts in a paramyxovirus (such as RSV or HPIV) RNA or coronavirus, other than SARS, RNA thus modulating the expression or activity of the RNA molecule. Targeting multiple binding sites on a single target RNA molecule can provide more effective modulation of the target RNA molecule. In another embodiment, the multitargeting interfering RNA of the invention forms stable interactions with at least two distinct binding sequences present in distinct genetic contexts on multiple pre-selected target RNA molecules wherein at least one of the binding sequences is present in paramyxovirus RNA or coronavirus, other than SARS, RNA, thus modulating the expression or activity of paramyxovirus RNA or coronavirus, other than SARS, RNA and other pre-selected target RNA molecules. One target may include another virus or a host protein. Targeting multiple target RNA molecules represents an alternative to the prototypical one-drug, one-target approach. In considering the complexity of biological systems, designing a drug selective for multiple targets will lead to new and more effective medications for a variety of diseases and disorders.

The targeted sequences may encode either structural or non-structural proteins or be non-coding RNA (eg NTR or UTR sequences or the like). They may also comprise any combination of the above.

In some embodiments, the multitargeting interfering RNA of the invention are designed to target one or more target RNA molecules in a first biological system and one or more target molecules in a second biological system that is infectious to the first biological system. In particular embodiments, the multitargeting interfering RNA of the invention are designed to target one or more host RNA molecules and one or more paramyxovirus RNA or coronavirus, other than SARS, RNA molecules. Therefore, the multitargeting interfering RNA molecule of the invention can, for example, decrease expression of IL-8 and modulate expression of RSV.

Such molecules, the skilled artisan will appreciate, can target multiple sites on a single RNA or multiple sites on two or more RNAs and are useful to decrease the expression of RSV or preferably RSV and one or more other targeted RNAs in an expression system.

In some embodiments, a given multitargeting interfering RNA will be more effective at modulating expression of one of several target RNAs than another. In other cases, the multitargeting interfering RNA will similarly affect all targets in one or more expression systems. Various factors can be responsible for causing variations in silencing or RNAi efficiency: (i) asymmetry of assembly of the RISC causing the passenger strand to enter more efficiently into the RISC than the guide strand; (ii) inaccessibility of the targeted segment on the target RNA molecule; (iii) a high degree of off-target activity by the interfering RNA; (iv) sequence-dependent variations for natural processing of RNA, and (v) the balance of the structural and kinetic effects described in (i) to (iv). See Hossbach et al. (2006), RNA Biology 3: 82-89. In designing a multitargeting interfering RNA molecule of the invention, special attention can be given to each of the listed factors to increase or decrease the RNAi efficiency on a given target RNA molecule.

Typically the multitargeting interfering RNA molecules of Formula(I) and Formula (II) will be designed as described in detail in co-pending international patent application nos. PCT/AU2006/001741 and PCT/AU2006/001750, the disclosures of which are incorporated herein by reference.

The step of obtaining the sequences for the selected target or targets is typically conducted by obtaining sequences from publicly available sources, such as the databases provided by the National Center For Biotechnology Information (NCBI) (through the National Institutes of Health (NIH) in the United States), the European Molecular Biology Laboratories (through the European Bioinformatics Institute throughout Europe) available on the World-Wide Web, or proprietary sources such as fee-based databases and the like. Sequences can also be obtained by direct determination. This may be desirable where a clinical isolate or an unknown gene is involved or of interest, for example, in a disease process. Either complete or incomplete sequences of a target RNA molecule can be used for the design of multitargeting interfering RNA of the invention.

It will be apparent to one skilled in the art that seed sites in RSV, or for example, common to RSV and IL-8, can be searched for in relevant databases such as the GenBank database hosted by the NCBI (www.ncbi.nlm.nih.gov) or the Ensembl database (www.ensembl.org). For example, sequences of RSV that could be used include GenBank Accession Number NC_{—}001781, gi:1912287 or AY353550. Sequences of IL-8 that could be used include the mRNA sequence of IL-8 GenBank Accession Number NM_{—}000584 or Ensembl Accession Number enst00000307407. Other sequences may be found for other host targets that it would be beneficial to target along with RSV.

Sequences of HPIV that could be used in VIROMIR design include GenBank Accession Number NC_{—}003461 (Type 1), NC_{—}003443 (Type 2), and NC_{—}001796 (Type 3). There are no full length complete genome sequences for either 4a or 4b, though there are individual Genbank accessions for each of the genes (except for the L gene of Type 4b). The sequences for individual genes from HPIV Type 4 include D10242 (encodes protein M, Type 4b), D10241 (protein M, Type 4a), EF088283 (protein L, Type 4a), EF088282 (protein F, Type 4a), EF088280 (protein P, Type 4a), EF088279 (protein N, Type 4a), E02727 (protein HN, Type 4a), E03305 (protein P, Type 4b), AB006958 (protein HN, Type 4b), D49822 (protein F, Type 4b) and M32983 (protein N, Type 4b).

Sequences that could be used to develop interfering RNA molecules targeting coronaviruses include human coronavirus 229E (GenBank Accession Number NC_{—}002645) and NL63 (NM_{—}200324) as well as transmissible gastroenteritis virus (NC_{—}002306), Human Coronavirus OC43 (NC005147) and HKU1 (NC_{—}006577).

In certain embodiments, the designed multitargeting interfering RNA molecule can be modified, for example, i) to improve the incorporation of the guide strand of the multitargeting interfering RNA molecule into the RNA induced silencing complex (RISC); ii) to obtain approximately equal loading of each strand into the RISC so that each strand in the duplex can act as a guide strand; iii) to increase or decrease the modulation of the expression of at least one target RNA molecule; iii) to decrease stress or inflammatory response when the multitargeting interfering RNA molecule is administered into a subject; or iv) any combination of i) to iii).

The skilled artisan will understand how to modify the RNA molecules either in the laboratory, or preferably in silico. In preferred embodiments the modifying step comprises one or more of altering, deleting, or introducing one or more nucleotide bases to create at least one mismatched base pair, wobble base pair, or terminal overhang, or to increase RISC mediated processing. Techniques for doing so are known in the art. Preferably the modifications are at least initially performed in silico, and the effects of such modifications can be readily tested against experimental parameters to determine which offer improved properties of the interfering RNA products.

As will be understood once a particular interfering RNA molecule is developed biological testing may also be used to exclude candidates with undesirable effects. For example, in RSV, those VIROMIRs that show stimulation of RSV replication and/or increase of IL-8 may be considered to be unsuitable because in RSV, downregulation of RSV replication and/or IL-8 is the desirable effect.

Candidate multitargeting interfering RNA are routinely synthesized as double-stranded RNA molecules with 19 by of complementarity and 3′ two nucleotide overhangs. For the guide strand (the strand with complementarity to the target RNAs and which is predicted to be incorporated into RISC), the two nucleotide overhangs are routinely designed to be complementary to the target RNAs, although dTdT or UU overhangs may also suit. The passenger strand (complementary to the guide strand) can be usually designed to include a 3′ two nucleotide UU overhang. However, other types and lengths of overhangs could be considered, as could “blunt-ended” duplexes. Candidate multitargeting interfering RNA can also be single-stranded molecules. In some embodiments (e.g. as for Formula (II)), both strands can be designed to act as guide strands. In some aspects of this invention X may have a length of 1, 2, 3 or more nucleotides.

When produced by an expression system such as a vector or plasmid, it is possible to assemble multiple multitargeting interfering RNAs into a single therapeutic product. Skilled artisans will realize that multiple multitargeting interfering RNAs can be co-expressed by several strategies, including but not limited to, expression of individual multitargeting interfering RNAs from multiple expression vectors (plasmid or viral), expression from multiple expression cassettes contained within a single vector, with each expression cassette containing a promoter, a single multitargeting interfering RNA and terminator. Multiple multitargeting interfering RNAs can also be generated through a single polycistronic transcript, which contains a series of multitargeting interfering RNAs.

The multiple multitargeting interfering RNAs can be expressed sequentially (sense/intervening loop/antisense) or expressed with the sense sequence of each multitargeting interfering RNA sequentially linked 5′ to 3′, joined directly or with intervening loop/spacer sequence, followed by the antisense sequence of each multitargeting interfering RNAs sequentially linked 5′ to 3′.

Assays using reporter constructs and appropriate viral replication models may be used to test multitargeting interfering RNAs of the invention. For example, in the first instance, multitargeting interfering RNAs may be tested in assays using reporter constructs comprising the target RNA sequence. Those multitargeting interfering RNA candidates selected using such an assay may then be tested in paramyxovirus or coronavirus replication models. Some non-limiting specific conditions used are outlined in the specific examples. Multitargeting interfering RNA that modulate target RNA expression or activity (e.g. viral replication) can then be studied further. Proteomic and microarray experiments may be used to assess off-target effects.

Candidate multitargeting interfering RNA molecules (e.g. VIROMIRs) could be tested in a number of in vitro and preclinical models well known in the art. For example, activity against RSV could be measured using a GFP-expressing transgenic clone as described elsewhere in this application. Animal models include the use of the American cotton rat for studies with RSV, influenza and measles among others.

Preferably, the candidate multitargeting interfering RNA are tested for non-specific toxic effects by, for example, direct assays of cell toxicity. Multitargeting interfering RNA are additionally assessed for their ability to repress the production of specific target proteins. Multitargeting interfering RNA demonstrating efficacy in this respect are then assessed for additional evidence of off-target effects.

The RNA molecule may be expressed from transcription units inserted into vectors. The vector may be a recombinant DNA or RNA vector, and includes DNA plasmids or viral vectors. The viral vectors expressing the multitargeting interfering RNA molecules can be constructed based on, but not limited to, adeno-associated virus, retrovirus, adenovirus, lentivirus or alphavirus.

Preferably the vector is an expression vector suitable for expression in a mammalian cell.

Methods which are well known to those skilled in the art can be used to construct expression vectors containing a sequence which encodes the multi target RNA molecule. These methods include in vitro recombinant DNA techniques, synthetic techniques and in vivo recombination or genetic recombination. Suitable routes of administration of the pharmaceutical composition of the present invention may, for example, include oral, rectal, transmucosal, or intestinal administration; parenteral delivery, including intramuscular, intravenous and subcutaneous injections.

Alternatively, the pharmaceutical composition may be administered in a local rather than systemic manner, for example, via injection of the pharmaceutical composition directly into a target organ or tissue, such as intramedullary, intrathecal, direct intraventricular, intraperitoneal, or intraocular injections, often in a depot or sustained release formulation. Intravesicular instillation and intranasal/inhalation delivery are other possible means of local administration as is direct application to the skin or affected area. Ex vivo applications are also envisaged.

Furthermore, the pharmaceutical composition of the present invention may be delivered in a targeted delivery system, for example, in a liposome coated with target cell-specific antibody. The liposomes will be targeted to and taken up selectively by the target cell. Other delivery strategies include, but are not limited to, dendrimers, polymers, electroporation, nanoparticles and ligand conjugates of the RNA.

The multitargeting interfering RNA molecules of the invention can be added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers.

In another aspect, the invention provides biological systems containing one or more multitargeting interfering RNA molecules of this invention. The invention also provides a vector comprising a nucleotide sequence that encodes the multitargeting interfering RNA molecule of the invention. In a particular embodiment, the vector is viral, for example, derived from a virus selected from the group consisting of an adeno-associated virus, a retrovirus, an adenovirus, a lentivirus, and an alphavirus. The multitargeting interfering RNA can be a short hairpin RNA molecule, which can be expressed from a vector of the invention. The invention further provides a pharmaceutical composition comprising a multitargeting interfering RNA molecule of the invention and an acceptable carrier. In particular embodiments, the pharmaceutical composition comprises a vector for a multitargeting interfering RNA molecule of the invention.

In another general aspect, the present invention provides a method of inducing RNA interference in a biological system, which comprises the step of introducing a multitargeting interfering RNA molecule of the invention into the biological system.

In a particular embodiment, the present invention provides a method of inducing RNA interference in a biological system, comprising the steps of: (a) selecting a set of target RNA molecules; (b) designing a multitargeting interfering RNA molecule that can form stable interactions with at least two binding sequences present in distinct genetic contexts in the set of target RNA molecules wherein at least one of the binding sequences is present in an RNA of a paramyxovirus or coronavirus, other than SARS, which results in respiratory impairment or disease, including an RSV or HPIV, (c) producing the multitargeting interfering RNA molecule; and (d) administering the multitargeting interfering RNA molecule into the biological system, whereby the multitargeting interfering RNA molecule forms stable interactions with the binding sequences present in distinct genetic contexts in the set of target RNA molecules, and thus induces RNA interference of the target RNA molecules.

In another particular embodiment, the present invention provides a method of treating respiratory impairment or disease due to a paramyxovirus, including a RSV or HPIV, or a coronavirus, other than SARS, in a subject, the method comprising the steps of: (a) selecting a set of target RNA molecules, wherein the modulation in expression of the target RNA molecules is potentially therapeutic for the treatment of the disease or condition; (b) designing a multitargeting interfering RNA molecule that can form stable interactions with at least two binding sequences present in distinct genetic contexts in the set of target RNA molecules wherein at least one of the binding sequences is present in an paramyxovirus RNA or coronavirus, other than SARS, RNA; (c) producing the multitargeting interfering RNA molecule; (d) administering the multitargeting interfering RNA molecule into the subject, whereby the multitargeting interfering RNA molecule forms stable interactions with the binding sequences present in distinct genetic contexts in the set of target RNA molecules, and thus induces modulation of expression of the target RNA molecules.

The use of single- or double-stranded RNA compounds that can target multiple sites within the paramyxovirus, including RSV or HPIV, or coronavirus, other than SARS, is also provided herein. The multitargeting interfering RNA molecules that target multiple sites in the paramyxovirus genome or coronavirus genome, other than SARS, of one or multiple isolates of the virus are sometimes referred herein as “VIROMIRs”. Targeting repeated sequence elements in viral genomes is an attractive approach for viral therapy. Such multitargeting is calculated to create a formidable hurdle to the emergence of resistant clones, which would require multiple, simultaneous, mutations. Also, multiple sites can be chosen to maximize coverage of sequence variations across a range of viral isolates. Elements can be identified computationally that are present in a pre-selected percentage of isolates, such as a majority or even the totality of known isolates, thereby ensuring maximal therapeutic benefit. Alternatively, isolates of greatest actual or projected clinical significance can be preferentially targeted. The design process can also facilitate development, manufacture, and ultimately administration of the therapeutic compounds. The additional targeting of one or more host proteins or other intermediates of the pathway involved in the pathogenesis of the viral disease (eg IL-8 in the case of RSV) can also be designed, as can targeting of another virus.

The RNA compounds of the present invention can be used to treat or prevent diseases in animals, in particular humans. The RNA compounds can be either cell-expressed into the relevant animal or human cell to derive the required effect or be administered as a chemically synthesized compound directly or indirectly by means of a delivery molecule or device.

Additionally, targeting of different isolates of the paramyxovirus or coronavirus, other than SARS,is envisaged. The multiple target sites can then be chosen to maximise coverage of sequence variations across a range of isolates.

The modulation of the target RNA molecule is determined in a suitable expression system, for example in vivo, in one or more suitable cells, or in an acellular or in vitro expression system such as are known in the art. Routine methods for measuring parameters of the transcription, translation, or other aspects of expression relating to RNA molecules are known in the art, and any such measurements are suitable for use herein.

The multitargeting RNAs in accordance with various aspects of the invention are useful to modulate the expression of one or more target RNA in an expression system wherein at least one target RNA is a paramyxovirus RNA or coronavirus, other than SARS, RNA. More preferably, they are used to reduce expression of one or more target RNA. Such decrease can occur directly or indirectly by any mechanism known in the art, or which is yet to be discovered, for the decrease of RNA expression as defined herein by an RNA. In some embodiments, they may completely eliminate expression of the one or more RNA targets. In some embodiments, a given RNA will be more effective at modulating expression of one of several target RNAs than another. In other cases, the RNA may similarly affect all targets in one or more expression systems.

In certain embodiments, for example where RISC is involved in the mechanism of action, the targeting of multiple disease-related transcripts with a single multitargeting interfering RNA makes optimal use of available RISC, in contrast to the administration of multiple siRNA molecules, which could saturate the available intracellular machinery.

Targeting multiple sites within the same viral (e.g. RSV or HPIV, or coronavirus, other than SARS,) RNA target sequence is also envisioned for the interfering RNAs provided herein, i.e. the multitargeting aspect is not limited to multiple targets within multiple target RNA molecules. Targeting multiple sites within the target viral RNA decreases the likelihood of such resistance arising, since at least two simultaneous mutations would be required for resistance. Therefore in certain embodiments, the multi-targeting approach used with multitargeting interfering RNAs can be directed to the generation of multiple hits against a single target viral RNA molecule, for example, to prevent escape mutants. Targeting of multiple sites within the same transcript may also produce synergistic effects on the inhibition of viral growth. Further, employing a mechanism or mechanisms requiring only partial complementarity with the target RNA molecule can decrease the possibility of developing resistance through single point mutation.

This invention will be better understood by reference to the examples that follow. Those skilled in the art will readily appreciate that these examples are only illustrative of the invention and not limiting.

### Example 1

Co-Suppression of RSV and Disease Implicated Host Protein (IL-8)

In the case of infectious diseases, VIROMIRs can be utilized to target both the genome of the infectious agent and one or more key host “drivers” of the disease. Example of host proteins involved in RSV infection include proteins involved in inflammation such as IL-8, receptors to which RSV can bind such as heparan sulphate, GTP-binding proteins such as RhoA, cytoskeletal proteins such as actin or profilin and heat shock proteins such as Hsp70 (Sugrue, 2006). For example, IL-8 is considered a major disease-associated factor in Respiratory Syncytial Virus (RSV) infection. RSV disease severity has been found to be strongly associated with the infants' inflammatory response, specifically interleukin-8 (IL-8) production in the airways (Smyth et al., 2002). This forms the rationale for developing VIROMIRs to target not only the infective agent itself (RSV) but also the host's immune response, IL-8, which exacerbates the morbidity and mortality from the disease.

It will be apparent to one skilled in the art that seed sites in RSV, or common to RSV and IL-8, can be searched for in relevant databases such as the GenBank database hosted by the NCBI (www.ncbi.nlm.nih.gov) or the Ensembl database (www.ensembl.org). For example sequences of RSV that could be used include GenBank Accession Number NC_{—}001781 or AY353550. Sequences of IL-8 that could be used include the mRNA sequence of IL-8 GenBank Accession Number NM_{—}000584 or Ensembl Accession Number enst00000307407). Other sequences may be found for other targets that it would be beneficial to target along with RSV.

We searched for seeds of nine nucleotides or greater in length that were common to the mRNA sequence of IL-8 (GenBank Accession Number NM_{—}000584) and the RSV genome of a RSV-eGFP variant used in in vitro studies, the sequence of which has 97% homology to the NCBI reference strain U39662. Identified seeds common to both IL-8 and RSV were as follows: ACAAACUUUC (SEQ ID NO: 6), AACCAUCUCACU (SEQ ID NO: 7), CAUAAAGACAU (SEQ ID NO: 86), UUAUCAAAGAA (SEQ ID NO: 1), AUUGAAUGG, GAACUGAGA, GUGAUAUUUG (SEQ ID NO: 87), UGUGGUAUC, UCAAGCAAAU (SEQ ID NO: 88), CAGAUGCAA, AUACAAGAU, UUCCUGGUUA (SEQ ID NO: 89), AUCCAGAAC, AUAUAAGGAUU (SEQ ID NO: 90), UAGCAAAAUUG (SEQ ID NO: 91), CAUCAUAACA (SEQ ID NO: 92), AAUUUAGCUGGA (SEQ ID NO: 2), GGAAGCACU, AUAAAUUUCAA (SEQ ID NO: 93), CAUCAAAUAU (SEQ ID NO: 3), GAUUGAAUA, AUAGUUAUA, UUAUUAGAUAA (SEQ ID NO: 4), UUAGAUAAAU (SEQ ID NO: 94), AUUUCAAUCA (SEQ ID NO: 95), UUGAUACUCC (SEQ ID NO: 5), ACUAACAAU, UCCUAGUUU, AGUUUGAUAC (SEQ ID NO: 96), AUUGCCAGC, GAAUAAUGA, ACAGCCAAA, AUUAGUAAU, UUUAUUAUGU (SEQ ID NO: 97), CAAAUAGAU, AAUAGAUUC, AUAAUAUUAU (SEQ ID NO: 98), AUAUGAAAC, AGGACAAGA, UACAUUAUU and CUCUGUGGU. Using these seeds, a set of VIROMIRs was designed (Table 1).

The VIROMIRs were synthesized commercially as RNA duplexes with 2 nucleotide overhangs on the 3′ end of the passenger and guide strands (Table 1). Wobble bases (G-U base pair) and mismatches were incorporated into a number of the VIROMIRs in order to facilitate loading of the molecule into the RISC complex. For example, RS003, RS005, RS006, RS011 and RS017 all contained wobble bases in the duplex molecule at the 5′ end of the guide strand and RS028 and RS029 contained an A/C mismatch at position 2 (5′) of the guide strand. The predicted binding of the VIROMIRs to their targets as determined using RNA Hybrid algorithm is shown in Table 2. A number of VIROMIRs were designed so the sequence complementarity to either IL-8 or RSV was maximized. For example RS001 and RS016 both target the same sites on RSV and IL-8, however RS016 was designed to have greater complementarity to the IL-8 sequence. VIROMIRS RS017, RS018, RS019, RS020 and RS021, which were designed using the same seed sites as RS003, RS007, RS008, RS010 and RS013 respectively were modified to have increased sequence complementarity to the RSV genome. In most cases, when present, X consists of 1 or 2 nucleotides, but in some cases X has a length of 3 or more nucleotides.

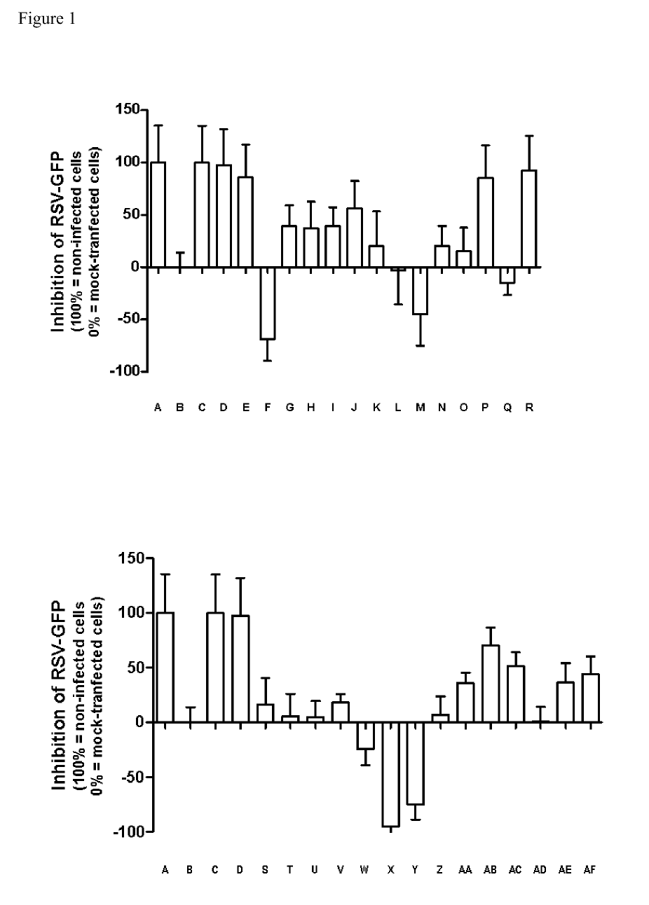

The RSV VIROMIRs were tested for their effect on RSV using A549 cells infected with the RSV-eGFP strain at an MOI of 0.01. A549 cells were plated on Day 1 in a 96 well plate at 4,000 cells/well (80 μL) in DMEM medium without antibiotics. The following day (Day 2), the cells were transfected with the VIROMIRs and control RSV siRNAs (siRSVP1 and siRSVP2; Table 3). These control siRNAs have been shown to significantly decrease RSV in mice when administered nasally (Bitko et al., 2005). For each well of a 96-well plate, 0.2 μL of a 20 μM stock of VIROMIR or siRNA was mixed with OptiMEM (10 μL final volume). This mixture was complexed for 20 minutes with 0.2 μL Lipofectamine2000 in 10 μL OptiMEM. The complex (20 μL) was added to cells in 80 μL DMEM medium so as to provide a final concentration of 40 nM dsRNA. Cells were infected on Day 3 by the addition of 100 μL of RSV-GFP to an MOI of 0.01. Two days later, fluorescence was measured, supernatants collected, cellular RNA extracted and RT-PCR performed on the human acidic ribosomal phosphoprotein PO (rplpo) transcript to ensure minimal toxicity. All treatments were performed in triplicate and the dsRNA were arranged across two plates with all positive (siRSVP1 and siRSVP2) and negative controls (untreated and mock-transfected cells) replicated on both plates. The sequences for the VIROMIRs and positive controls are shown in Tables 1 and 3, respectively. The positive control siRNAs for RSV (siRSVP1 and siRSVP2) suppressed RSV-GFP fluorescence by ˜100% (FIG. 1). Several VIROMIRs had profound activity against the virus with approximately 70% (RS026), 85% (RS001, RS012) and 90% (RS016) suppression (FIG. 1). A set of VIROMIRs had moderate activity against RSV with approximately 50% (RS006 and RS027), 40-45% (RS003, RS004, RS005 and RS031) and 35% (RS025 and RS030) inhibition.

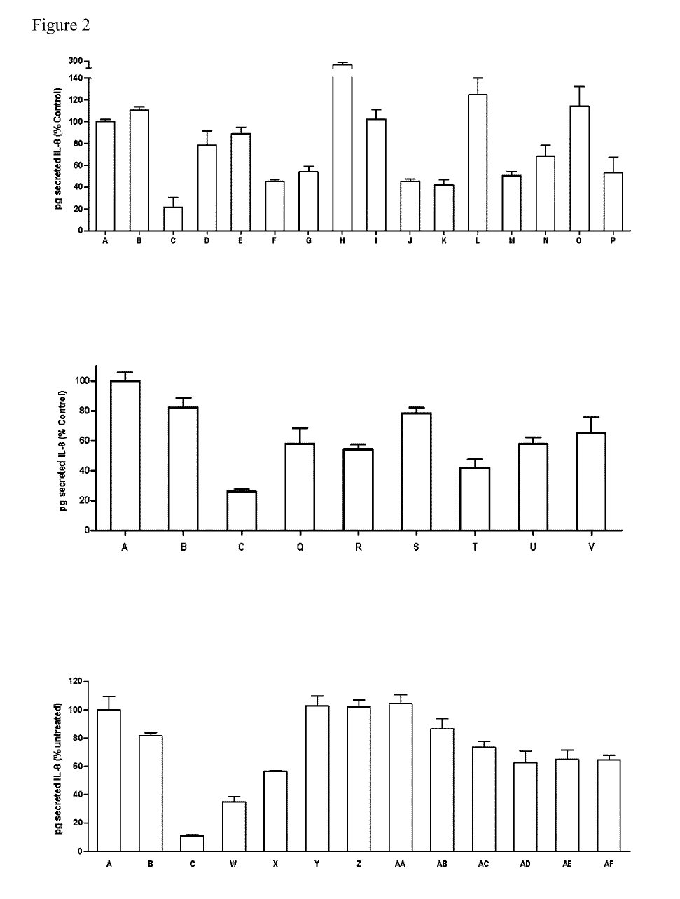

The RSV VIROMIRs were also tested for their effect on IL-8 secretion. A549 cells were plated on Day 1 in a 96 well plate at 4,000 cells/well (80 μL) in DMEM medium without antibiotics. The following day (Day 2), the cells were transfected with the VIROMIRs and a control IL-8 siRNA (siIL-8; Table 3). For each well of a 96-well plate, 0.2 μL of a 20 μM stock of VIROMIR or siRNA was mixed with OptiMEM (10 μL final volume). This mixture was complexed for 20 minutes with 0.2 μL Lipofectamine2000 in 10 μL OptiMEM. The complex (20 μL) was added to cells in 80 μL DMEM medium so as to provide a final concentration of 40 nM dsRNA. All treatments were performed in triplicate and the abundance of secreted IL-8 measured 72 h post-transfection using an IL-8 ELISA (R&D Systems). The control siRNA for IL-8 (siIL-8) suppressed IL-8 secretion by approximately 80% (FIG. 2). Several VIROMIRs suppressed production of IL-8 with approximately 60-65% inhibition (RS019 and RS022), 50-55% inhibition (RS003, RS007, RS008 and RS010), 40-45% inhibition (RS004, RS013, RS016, RS017, RS020 and RS023) and 30-35% inhibition (RS011, RS021, RS029, RS030 and RS031). RS001 and RS016, both of which were active against RSV, were related in that they target the same sites on RSV and IL-8. However, RS016 was designed to have greater complementarity to the IL-8 CDS sequence to achieve better repression of IL-8 than was seen with RS001. Transfection of ARPE-19 cells with 40 nM RS016 resulted in >40% inhibition of IL-8 72 h post-transfection while transfection with 40 nM RS001 resulted in 20% inhibition (FIG. 2). Examples of other VIROMIRs which suppressed both RSV and IL-8 include RS003 (40% and 55% respectively), RS004 (40% and 45% respectively), RS030 (35% and 35% respectively) and RS031 (45% and 35% respectively). In some cases in this viral replication assay, an apparent stimulation of viral replication was observed.

TABLE 1
List of VIROMIRs designed to target RSV and human IL-8.

| | | | | DUPLEX | | |
|---|---|---|---|---|---|---|
| | | | | (top = passenger | | |
| | | | | strand 5′ to 3′ | | |
| | | | | (SEQ ID NOS 37-65); | | |
| | | | | bottom = guide | | |
| | TARGET SEED | GUIDE STRAND | PASSENGER STRAND | strand 3′ to 5′ | IL-8 | RSV |
| VIROMIR | (5′ TO 3′) | (5′ TO 3′) | (5′ TO 3′) | (SEQ ID NOS 8-36)) | Target* | Target* |
| RS001 | UUAUCAAAGAA | UUCUUUGAUAAUAUUGGGGUG | CCCCAAUAUUAUCAAAGAAUU | CCCCAAUAUUAUCAAAGAAUU | CDS | P |
| | (SEQ ID NO: 1) | (SEQ ID NO: 8) | (SEQ ID NO: 37) | GUGGGGUUAUAAUAGUUUCUU | | |
| RS002 | GAACUGAGA | UCUCAGUUCAAUGAUAGGUUU | ACCUAUCAUUGAACUGAGAUU | ACCUAUCAUUGAACUGAGAUU | CDS | L |
| | | (SEQ ID NO: 9) | (SEQ ID NO: 38) | UUUGGAUAGUAACUUGACUCU | | |
| RS003 | UGUGGUAUC | GAUACCACACUGAAUGGGUUU | ACCCAUUCAGUGUGGUAUUUU | ACCCAUUCAGUGUGGUAUUUU | 3′UTR | L |
| | | (SEQ ID NO: 10) | (SEQ ID NO: 39) | UUUGGGUAAGUCACACCAUAG | | |
| RS004 | CAGAUGCAA | UUGGUUGCAUCUGCGAACCCU | GGUUCGCAGAUGCAACCAAUU | GGUUCGCAGAUGCAACCAAUU | 3′UTR | L |
| | | (SEQ ID NO: 11) | (SEQ ID NO: 40) | UCCCAAGCGUCUACGUUGGUU | | |
| RS005 | AUCCAGAAC | GUUCUGGAUUAUUCAUGGUAC | ACCAUGAAUAAUCCAGAAUUU | ACCAUGAAUAAUCCAGAAUUU | 3′UTR | G |
| | | (SEQ ID NO: 12) | (SEQ ID NO: 41) | CAUGGUACUUAUUAGGUCUUG | | |
| RS006 | AUCCAGAAC | UGUUCUGGAUUAUUCAUGGUA | CCAUGAAUAAUCCAGAAUAUU | CCAUGAAUAAUCCAGAAUAUU | 3′UTR | G |
| | | (SEQ ID NO: 13) | (SEQ ID NO: 42) | AUGGUACUUAUUAGGUCUUGU | | |
| RS007 | AAUUUAGCUGGA | AUUUCCAGCUAAAUUUGACUU | GUCAAAUUUAGCUGGAAAUUU | GUCAAAUUUAGCUGGAAAUUU | 3′UTR | L |
| | (SEQ ID NO: 2) | (SEQ ID NO: 14) | (SEQ ID NO: 43) | UUCAGUUUAAAUCGACCUUUA | | |
| RS008 | CAUCAAAUAU | UAUUUGAUGGAUAAAUAAGUA | CUUAUUUAUCCAUCAAAUAUU | CUUAUUUAUCCAUCAAAUAUU | 3′UTR | L |
| | (SEQ ID NO: 3) | (SEQ ID NO: 15) | (SEQ ID NO: 44) | AUGAAUAAAUAGGUAGUUUAU | | |
| RS009 | GAUUGAAUA | UUAUUCAAUCUUUGAUUCAUC | UGAAUCAAAGAUUGAAUAAUU | UGAAUCAAAGAUUGAAUAAUU | 3′UTR | L |
| | | (SEQ ID NO: 16) | (SEQ ID NO: 45) | CUACUUAGUUUCUAACUUAUU | | |
| RS010 | UUAUUAGAUAA | UUUAUCUAAUAAUUCAUCAUU | UGAUGAAUUAUUAGAUAAAUU | UGAUGAAUUAUUAGAUAAAUU | 3′UTR | L |
| | (SEQ ID NO: 4) | (SEQ ID NO: 17) | (SEQ ID NO: 46) | UUACUACUUAAUAAUCUAUUU | | |
| RS011 | UUGAUACUCC | AUUGGGAGUAUCAAAUCUAUC | UAGAUUUGAUACUCCUAAUUU | UAGAUUUGAUACUCCUAAUUU | 3′UTR | L |
| | (SEQ ID NO: 5) | (SEQ ID NO: 18) | (SEQ ID NO: 47) | CUAUCUAAACUAUGAGGGUUA | | |
| RS012 | GAAUAAUGA | AUUCAUUAUUCGCUAAUUCAA | GAAUUAGCGAAUAAUGAAUUU | GAAUUAGCGAAUAAUGAAUUU | 3′UTR | L |
| | | (SEQ ID NO: 19) | (SEQ ID NO: 48) | AACUUAAUCGCUUAUUACUUA | | |
| RS013 | AUUAGUAAU | AUUACUAAUUAUGACUGUGGA | CACAGUCAUAAUUAGUAAUUU | CACAGUCAUAAUUAGUAAUUU | 3′UTR | L |
| | | (SEQ ID NO: 20) | (SEQ ID NO: 49) | AGGUGUCAGUAUUAAUCAUUA | | |
| RS016 | UUAUCAAAGAA | UUCUUUGAUAAAUUUGGGCUG | GCCCAAAUUUAUCAAAGAAUU | GCCCAAAUUUAUCAAAGAAUU | CDS | P |
| | (SEQ ID NO: 1) | (SEQ ID NO: 21) | (SEQ ID NO: 50) | GUCGGGUUUAAAUAGUUUCUU | | |
| RS017 | UGUGGUAUC | GAUACCACAUGGUUAGGGUUU | ACCCUAACCAUGUGGUAUUUU | ACCCUAACCAUGUGGUAUUUU | 3′UTR | L |
| | | (SEQ ID NO: 22) | (SEQ ID NO: 51) | UUUGGGAUUGGUACACCAUAG | | |
| RS018 | AAUUUAGCUGGA | AUGUCCAGCUAAAUUGUACUU | GUACAAUUUAGCUGGACAUUU | GUACAAUUUAGCUGGACAUUU | 3′UTR | L |
| | (SEQ ID NO: 2) | (SEQ ID NO: 23) | (SEQ ID NO: 52) | UUCAUGUUAAAUCGACCUGUA | | |
| RS019 | CAUCAAAUAU | UAUUUGAUGUUUAUUGAAGAC | CUUCAAUAAACAUCAAAUAUU | CUUCAAUAAACAUCAAAUAUU | 3′UTR | L |
| | (SEQ ID NO: 3) | (SEQ ID NO: 24) | (SEQ ID NO: 53) | CAGAAGUUAUUUGUAGUUUAU | | |
| RS020 | UUAUUAGAUAA | UUUAUCUAAUAAUGUAUGACA | UCAUACAUUAUUAGAUAAAUU | UCAUACAUUAUUAGAUAAAUU | 3′UTR | L |
| | (SEQ ID NO: 4) | (SEQ ID NO: 25) | (SEQ ID NO: 54) | ACAGUAUGUAAUAAUCUAUUU | | |
| RS021 | AUUAGUAAU | AUUACUAAUGUUGCUGUGCAU | GCACAGCAACAUUAGUAAUUU | GCACAGCAACAUUAGUAAUUU | 3′UTR | L |
| | | (SEQ ID NO: 26) | (SEQ ID NO: 55) | UACGUGUCGUUGUAAUCAUUA | | |
| RS022 | AUAGUUAUA | UAUAACUAUUCAAUCGGAGUU | CUCCGAUUGAAUAGUUAUAUU | CUCCGAUUGAAUAGUUAUAUU | 3′UTR | F UTR |
| | | (SEQ ID NO: 27) | (SEQ ID NO: 56) | UUGAGGCUAACUUAUCAAUAU | | |
| RS023 | AUAGUUAUA | UAUAACUAUAAACUAGGUGAU | CACCUAGUUUAUAGUUAUAUU | CACCUAGUUUAUAGUUAUAUU | 3′UTR | F UTR |
| | | (SEQ ID NO: 28) | (SEQ ID NO: 57) | UAGUGGAUCAAAUAUCAAUAU | | |
| RS024 | ACAAACUUUC | GAAAGUUUGUGUGAUAUGGAG | CCAUAUCACACAAACUUUCUU | CCAUAUCACACAAACUUUCUU | 5′UTR | M UTR |
| | (SEQ ID NO: 6) | (SEQ ID NO: 29) | (SEQ ID NO: 58) | GAGGUAUAGUGUGUUUGAAAG | | |
| RS025 | ACAAACUUUC | GAAAGUUUGUGUCUAUUGGCU | CCAAUAGACACAAACUUUCUU | CCAAUAGACACAAACUUUCUU | 5′UTR | M UTR |
| | (SEQ ID NO: 6) | (SEQ ID NO: 30) | (SEQ ID NO: 59) | UCGGUUAUCUGUGUUUGAAAG | | |
| RS026 | AACCAUCUCACU | AGUGAGAUGGUUUCUUCAUGA | AUGAAGAAACCAUCUCACUUU | AUGAAGAAACCAUCUCACUUU | 5′UTR | F UTR |
| | (SEQ ID NO: 7) | (SEQ ID NO: 31) | (SEQ ID NO: 60) | AGUACUUCUUUGGUAGAGUGA | | |
| RS027 | AACCAUCUCACU | AGUGAGAUGGUUUAUAGCGGU | CGCUAUAAACCAUCUCACUUU | CGCUAUAAACCAUCUCACUUU | 5′UTR | F UTR |
| | (SEQ ID NO: 7) | (SEQ ID NO: 32) | (SEQ ID NO: 61) | UGGCGAUAUUUGGUAGAGUGA | | |
| RS028 | CUCUGUGGU | ACCACAGAGGGUUGGUUGUUU | ACAACCAACCCUCUGUGAUUU | ACAACCAACCCUCUGUGAUUU | 3′UTR | M UTR |
| | | (SEQ ID NO: 33) | (SEQ ID NO: 62) | UUUGUUGGUUGGGAGACACCA | | |
| RS029 | CUCUGUGGU | ACCACAGAGGGUGGGUGGUUA | ACCACCCACCCUCUGUGAUUU | ACCACCCACCCUCUGUGAUUU | CDS/3′ | M UTR |
| | | (SEQ ID NO: 34) | (SEQ ID NO: 63) | AUUGGUGGGUGGGAGACACCA | UTR | |
| RS030 | CUCUGUGGU | AACCACAGAGGGUUGGUUGUU | CAACCAACCCUCUGUGGUUUU | CAACCAACCCUCUGUGGUUUU | 3′UTR | M UTR |
| | | (SEQ ID NO: 35) | (SEQ ID NO: 64) | UUGUUGGUUGGGAGACACCAA | | |
| RS031 | CUCUGUGGU | AACCACAGAGGGUGGGUGGUU | CCACCCACCCUCUGUGGUUUU | CCACCCACCCUCUGUGGUUUU | 3′UTR | M UTR |
| | | (SEQ ID NO: 36) | (SEQ ID NO: 65) | UUGGUGGGUGGGAGACACCAA | | |

*RSV targets: CDS, coding sequence; P, accessory phosphoprotein; L, large viral protein; G, attachment protein; M, membrane matrix proteins; F, fusion protein; UTR, untranslated region

TABLE 2

Predicted binding of VIROMIRs (lower sequence)
targeting RSV and IL-8 (upper sequence).

| | | |
|---|---|---|
| VIROMIR | Target binding RSV (RNA Hybrid) | Target Binding IL-8 (RNA Hybrid) |

| | | |
|---|---|---|
| RS001 | (SEQ ID NO: 149) | (SEQ ID NO: 163) |
| | target 5′ A G A 3′ | target 5′ C AU C 3′ |
| | A CCCA AUUAUCAAAGAA | CACCCCAA UUAUCAAAGAA |
| | U GGGU UAAUAGUUUCUU | GUGGGGUU AAUAGUUUCUU |
| | 3′ G G UA 5′ | 3′ AU 5′ |
| | (SEQ ID NO: 8) | (SEQ ID NO: 8) |
| RS002 | (SEQ ID NO: 150) | (SEQ ID NO: 164) |
| | target 5′ U ACAA AAAG C 3′ | target 5′ C AA G 3′ |
| | AGGCC AUUA GAACUGAGA | AAAUUUAUCA GAACUGAGA |
| | UUUGG UAGU CUUGACUCU | UUUGGAUAGU CUUGACUCU |
| | 3′ A AA 5′ | 3′ AA 5′ |
| | (SEQ ID NO: 9) | (SEQ ID NO: 9) |
| RS003 | (SEQ ID NO: 151) | (SEQ ID NO: 165) |
| | target 5′ C UAA A 3′ | target 5′ A UC C 3′ |
| | AACCC UCA UGUGGUAUC | AAAUUCAUUC UGUGGUAUC |
| | UUGGG AGU ACACCAUAG | UUUGGGUAAG ACACCAUAG |
| | 3′ U UA C 5′ | 3′ UC 5′ |
| | (SEQ ID NO: 10) | (SEQ ID NO: 10) |
| RS004 | (SEQ ID NO: 152) | (SEQ ID NO: 166) |
| | target 5′ A C 3′ | target 5′ U G A G 3′ |
| | CAGAUGCAACCAA | AGGGUU C CAGAUGCAAU CAA |
| | GUCUACGUUGGUU | UCCCAA G GUCUACGUUG GUU |
| | 3′ UCCCAAGC 5′ | 3′ C 5′ |
| | (SEQ ID NO: 11) | (SEQ ID NO: 11) |
| RS005 | (SEQ ID NO: 153) | (SEQ ID NO: 167) |
| | target 5′ C CAG U 3′ | target 5′ U AU A 3′ |
| | UACCA GA AAUCCAGAAC | GUACCAUGAA AUCCAGAAC |
| | AUGGU CU UUAGGUCUUG | CAUGGUACUU UAGGUCUUG |
| | 3′ C A UA 5′ | 3′ AU 5′ |
| | (SEQ ID NO: 12) | (SEQ ID NO: 12) |
| RS006 | (SEQ ID NO: 154) | (SEQ ID NO: 168) |
| | target 5′ C CAG U 3′ | target 5′ G AU U 3′ |
| | ACCA GAA AUCCAGAAC | UACCAUGAA AUCCAGAACA |
| | UGGU CUU UAGGUCUUG | AUGGUACUU UAGGUCUUGU |
| | 3′ A A AU U 5′ | 3′ AU 5′ |
| | (SEQ ID NO: 13) | (SEQ ID NO: 13) |
| RS007 | (SEQ ID NO: 155) | (SEQ ID NO: 169) |
| | target 5′ U ACU C 3′ | target 5′ A C 3′ |
| | AAGU AAUUUAGCUGGA | AAGUCAAAUUUAGCUGGAAAU |
| | UUCA UUAAAUCGACCU | UUCAGUUUAAAUCGACCUUUA |
| | 3′ GU UUA 5′ | 3′ 5′ |
| | (SEQ ID NO: 14) | (SEQ ID NO: 14) |
| RS008 | (SEQ ID NO: 156) | (SEQ ID NO: 170) |
| | target 5′ A U 3′ | target 5′ G AG U 3′ |
| | CAUCAAAUA | UAUUUAUUUA CAUCAAAUA |
| | GUAGUUUAU | AUGAAUAAAU GUAGUUUAU |
| | 3′ AUGAAUAAAUAG 5′ | 3′ AG 5′ |
| | (SEQ ID NO: 15) | (SEQ ID NO: 15) |
| RS009 | (SEQ ID NO: 157) | (SEQ ID NO: 171) |
| | target 5′ A U G 3′ | target 5′ A UU U 3′ |
| | GAUGA AGAUUGAAUAA | GAUGAAUCA GAUUGAAUAG |
| | CUACU UCUAACUUAUU | CUACUUAGU CUAACUUAUU |
| | 3′ UAGUU 5′ | 3′ UU 5′ |
| | (SEQ ID NO: 16) | (SEQ ID NO: 16) |
| RS010 | (SEQ ID NO: 158) | (SEQ ID NO: 172) |
| | target 5′ U UCAUAC A 3′ | target 5′ A UU U 3′ |
| | AUG AUUAUUAGAUAAG | AAUGAUG UUAUUAGAUAAA |
| | UAC UAAUAAUCUAUUU | UUACUAC AAUAAUCUAUUU |
| | 3′ U UACU 5′ | 3′ UU 5′ |
| | (SEQ ID NO: 17) | (SEQ ID NO: 17) |
| RS011 | (SEQ ID NO: 159) | (SEQ ID NO: 173) |
| | target 5′ A UA U 3′ | target 5′ C C 3′ |
| | GAUAG UUGAUACUCCUAAU | UAG UUUGAUACUCCCAGU |
| | CUAUC AACUAUGAGGGUUA | AUC AAACUAUGAGGGUUA |
| | 3′ UA 5′ | 3′ CU U 5′ |
| | (SEQ ID NO: 18) | (SEQ ID NO: 18) |
| RS012 | (SEQ ID NO: 160) | (SEQ ID NO: 174) |
| | target 5′ A A 3′ | target 5′ G CG U 3′ |
| | UGAAUAAUGAAU | UUGAAUUA GAAUAAUGAGU |
| | GCUUAUUACUUA | AACUUAAU CUUAUUACUUA |
| | 3′ AACUUAAUC 5′ | 3′ CG 5′ |
| | (SEQ ID NO: 19) | (SEQ ID NO: 19) |
| RS013 | (SEQ ID NO: 161) | (SEQ ID NO: 175) |
| | target 5′ G GC A 3′ | target 5′ C AU U 3′ |
| | UAUAG CA AUUAGUAAU | UCCACAGUCA AUUAGUAAU |
| | GUGUC GU UAAUCAUUA | AGGUGUCAGU UAAUCAUUA |
| | 3′ AG A AU 5′ | 3′ AU 5′ |
| | (SEQ ID NO: 20) | (SEQ ID NO: 20) |
| RS016 | (SEQ ID NO: 149) | (SEQ ID NO: 163) |
| | target 5′ A A 3′ | target 5′ C C C 3′ |
| | AGCCCAA UUAUCAAAGAA | CA CCCAAAUUUAUCAAAGAA |
| | UCGGGUU AAUAGUUUCUU | GU GGGUUUAAAUAGUUUCUU |
| | 3′ G UA 5′ | 3′ C 5′ |
| | (SEQ ID NO: 21) | (SEQ ID NO: 21) |
| RS017 | (SEQ ID NO: 162) | (SEQ ID NO: 165) |
| | target 5′ C U 3′ | target 5′ A AUU C C 3′ |
| | AACCCUAAUCAUGUGGUAUC | AAAUUC CU UGUGGUAUC |
| | UUGGGAUUGGUACACCAUAG | UUUGGG GG ACACCAUAG |
| | 3′ U 5′ | 3′ AUU U 5′ |
| | (SEQ ID NO: 22) | (SEQ ID NO: 22) |
| RS018 | (SEQ ID NO: 176) | (SEQ ID NO: 191) |
| | target 5′ U U U 3′ | target 5′ A CA A 3′ |
| | AAGUAC AAUUUAGCUGGACAU | AAGU AAUUUAGCUGGA |
| | UUCAUG UUAAAUCGACCUGUA | UUCA UUAAAUCGACCU |
| | 3′ 5′ | 3′ UG GUA 5′ |
| | (SEQ ID NO: 23) | (SEQ ID NO: 23) |
| RS019 | (SEQ ID NO: 177) | (SEQ ID NO: 192) |
| | target 5′ A U 3′ | target 5′ A UU U 3′ |
| | CAAUGGACAUCAAAUA | UUUA UAAGCAUCAAAUA |
| | GUUAUUUGUAGUUUAU | AAGU AUUUGUAGUUUAU |
| | 3′ CAGAA 5′ | 3′ CAG U 5′ |
| | (SEQ ID NO: 24) | (SEQ ID NO: 24) |
| RS020 | (SEQ ID NO: 178) | (SEQ ID NO: 193) |
| | target 5′ A A 3′ | target 5′ A U 3′ |
| | UGUCAUACAUUAUUAGAUAAG | UGUU UUAUUAGAUAAA |
| | ACAGUAUGUAAUAAUCUAUUU | ACAG AAUAAUCUAUUU |
| | 3′ 5′ | 3′ UAUGU 5′ |
| | (SEQ ID NO: 25) | (SEQ ID NO: 25) |
| RS021 | (SEQ ID NO: 179) | (SEQ ID NO: 194) |
| | target 5′ U A 3′ | target 5′ C U U 3′ |
| | AUGUAUAGCAGCAUUAGUAAU | CACAG CAAUAUUAGUAAU |
| | UACGUGUCGUUGUAAUCAUUA | GUGUC GUUGUAAUCAUUA |
| | 3′ 5′ | 3′ UAC 5′ |
| | (SEQ ID NO: 26) | (SEQ ID NO: 26) |
| RS022 | (SEQ ID NO: 180) | (SEQ ID NO: 195) |
| | target 5′ A UAGUUU U 3′ | target 5′ A A 3′ |
| | GAUUCC AUAGUUAUA | UUGAUUGAAUAGUUAUA |
| | UUGAGG UAUCAAUAU | GGCUAACUUAUCAAUAU |
| | 3′ CUAACU 5′ | 3′ UUGA 5′ |
| | (SEQ ID NO: 27) | (SEQ ID NO: 27) |
| RS023 | (SEQ ID NO: 181) | (SEQ ID NO: 196) |
| | target 5′ U U 3′ | target 5′ A GAUUGA A 3′ |
| | CCUAGUUUAUAGUUAUA | AUCAUU AUAGUUAUA |
| | GGAUCAAAUAUCAAUAU | UAGUGG UAUCAAUAU |
| | 3′ UAGU 5′ | 3′ AUCAAA 5′ |
| | (SEQ ID NO: 28) | (SEQ ID NO: 28) |
| RS024 | (SEQ ID NO: 182) | (SEQ ID NO: 197) |
| | target 5′ G A U 3′ | target 5′ AG A 3′ |
| | UU AU UCAUACAAACUUUC | CUCCAUA GCACAAACUUUC |
| | AG UA AGUGUGUUUGAAAG | GAGGUAU UGUGUUUGAAAG |
| | 3′ G G U 5′ | 3′ AG 5′ |
| | (SEQ ID NO: 29) | (SEQ ID NO: 29) |
| RS025 | (SEQ ID NO: 183) | (SEQ ID NO: 198) |
| | target 5′ U UC U 3′ | target 5′ U UA A 3′ |
| | GUUAAU AUACAAACUUUC | CCA AGGCACAAACUUUC |
| | CGGUUA UGUGUUUGAAAG | GGU UCUGUGUUUGAAAG |
| | 3′ U UC 5′ | 3′ UC UA 5′ |
| | (SEQ ID NO: 30) | (SEQ ID NO: 30) |
| RS026 | (SEQ ID NO: 184) | (SEQ ID NO: 199) |
| | target 5′ C CUAU U 3′ | target 5′ A CG G 3′ |
| | UCAU AAACCAUCUCACU | C GAAGGAACCAUCUCACU |
| | AGUA UUUGGUAGAGUGA | G CUUCUUUGGUAGAGUGA |
| | 3′ CUUC 5′ | 3′ A UA 5′ |
| | (SEQ ID NO: 31) | (SEQ ID NO: 31) |
| RS027 | (SEQ ID NO: 185) | (SEQ ID NO: 200) |
| | target 5′ A CUCAU U 3′ | target 5′ C GA G G 3′ |
| | ACU CUAUAAACCAUCUCACU | ACCG A GAACCAUCUCACU |
| | UGG GAUAUUUGGUAGAGUGA | UGGC U UUUGGUAGAGUGA |
| | 3′ C 5′ | 3′ GA A 5′ |
| | (SEQ ID NO: 32) | (SEQ ID NO: 32) |
| RS028 | (SEQ ID NO: 186) | (SEQ ID NO: 201) |
| | target 5′ C CAA A U 3′ | target 5′ A C A 3′ |
| | ACAAUCA AC CUCUGUGGU | AAUU AUUCUCUGUGGU |
| | UGUUGGU UG GAGACACCA | UUGG UGGGAGACACCA |
| | 3′ UU G 5′ | 3′ UUUG U 5′ |
| | (SEQ ID NO: 33) | (SEQ ID NO: 33) |
| RS029 | (SEQ ID NO: 187) | (SEQ ID NO: 202) |
| | target 5′ C AA A U 3′ | target 5′ A AA A 3′ |
| | AAUCAC AC CUCUGUGGU | AA AUUCAUUCUCUGUGGU |
| | UUGGUG UG GAGACACCA | UU UGGGUGGGAGACACCA |
| | 3′ A GG G 5′ | 3′ A GG 5′ |
| | (SEQ ID NO: 34) | (SEQ ID NO: 34) |
| RS030 | (SEQ ID NO: 188) | (SEQ ID NO: 201) |
| | target 5′ C CAA A C 3′ | target 5′ A C A 3′ |
| | ACAAUCA AC CUCUGUGGUU | AAUU AUUCUCUGUGGU |
| | UGUUGGU UG GAGACACCAA | UUGG UGGGAGACACCA |
| | 3′ U G 5′ | 3′ UUG U A 5′ |
| | (SEQ ID NO: 35) | (SEQ ID NO: 35) |
| RS031 | (SEQ ID NO: 189) | (SEQ ID NO: 203) |
| | target 5′ C AA A C 3′ | target 5′ A A 3′ |
| | AAUCAC AC CUCUGUGGUU | AUUCAUUCUCUGUGGU |
| | UUGGUG UG GAGACACCAA | UGGGUGGGAGACACCA |
| | 3′ GG G 5′ | 3′ UUGG A 5′ |
| | (SEQ ID NO: 36) | (SEQ ID NO: 36) |

TABLE 3
Positive control siRNAs for RSV.

| | | |
|---|---|---|
| GUIDE STRAND (5′ to 3′) | PASSENGER STRAND (5′ TO 3′ | Reference |

| | | | |
|---|---|---|---|
| siRSVP1 | UCUUGCAGUUAUAUUAUCGdTdT | CGAUAAUAUAACAGCAAGAdTdT | Bitko, et |
| | (SEQ ID NO: 72) | (SEQ ID NO: 76) | al., 2005 |
| siRSVP2 | AUUAUCACUUGGUGUAGGGdTdT | CCCUACACCAAGUGAUAAUdTdT | Bitko, et |
| | (SEQ ID NO: 73) | (SEQ ID NO: 77) | al., 2005 |
| siIL-8 | GAUGGUUCCUUCCGGUGGUdTdT | ACCACCGGAAGGAACCAUCdTdT | Geno- |
| | (SEQ ID NO: 74) | (SEQ ID NO: 78) | spectra |
| siGC47 | GUCUGCGAUCGCAUACAAUdTdT | AUUGUAUGCGAUCGCAGACdTdT | Dharma- |
| | (SEQ ID NO: 75) | (SEQ ID NO: 79) | con |

A second assay to test the effect of the VIROMIRs on RSV was utilized. In this assay a short RSV target sequence (<200 nucleotides) encoding part of Accessory Phosphoprotein (P) or the UTR of the viral fusion protein (F) was synthesized by Genscript Corporation (NJ, USA) and inserted into a GFP reporter plasmid (modified pd4-eGFP-N1 vector from Clontech). The two inserts (shown below) contained either a part of the sequence for Accessory phosphoprotein (P; PD1) or the UTR of the viral fusion protein (F; PD2) with an introduced XhoI and SacII site at the 5′ and 3′ ends, respectively (italicised). The mammalian-preferred stop codon, TGA, is in the three reading frames, immediately following the XhoI site and is indicated in bold.

| SEQUENCE FOR PD1 (5′ to 3′): | |
|---|---|
| (SEQ ID NO: 80) | |
| 1 | GGCTCGAGTG ATTGATTGAA CAAGCCCAAT TATCAAAGAA AACCTCTAGT |
| 51 | AAGTTTCAAA GAAGACCCTA CACCAAGTGA TAATCCCTTT TCTAAACTAT |
| 101 | ACAAAGAAAC CATAGAAACA TTTGATAACA ATGAAGAAGA ATCCAGCTAT |
| 151 | TCATACGAAG AAATAAATGA TCAGACAAAC GATAATATAA CAGCAAGATT |
| 201 | CCGCGGTT 208 |
| SEQUENCE FOR PD2 (5′ to 3′): | |
| (SEQ ID NO: 81) | |
| 1 | GGCTCGAGTG ATTGATTGAA TAAAAATAGC ACCTAATCAT GTTCTTACAA 50 |
| 51 | TGGTTTACTA TCTGCTCATA GACAACCCAT CTGTCATTGG ATTTTCTTAA 100 |
| 101 | AATCTGAACT TCATCGAAAC TCTCATCTAT AAACCATCTC ACTTACACTA 150 |
| 151 | TTTAAGTAGA TTCCTAGTTT ATAGTTATAT AAACCGCGGT T 191 |

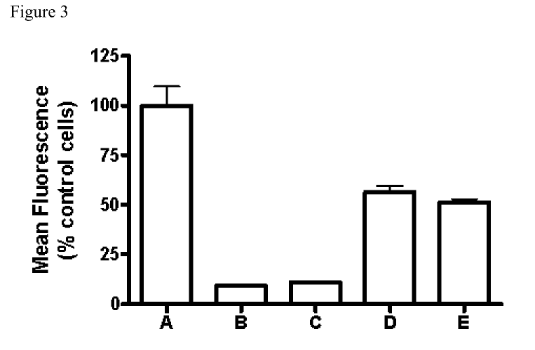

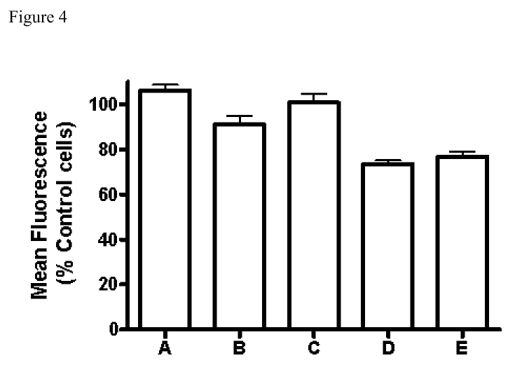

The effect of the VIROMIRs on GFP expression was determined using PD1 for RS001 and RS016 and using PD2 for RS022, RS023, RS026 and RS027. A549 cells in 6-well plates (1.2E+05 cells/well) were transfected with up to 750 ng plasmid+300 ng RNA duplex (0 ng RNA duplex in untransfected cells) using 5 μL Lipofectamine2000 in 250 μL OptiMEM in a total transfection volume of 2 mL. Fresh DMEM media was added 5 hours post-transfection and eGFP expression analysed by FACS 48 hours post-transfection. The two positive control siRNAs (siRSVP1 and siRSVP2) against RSV successfully inhibited eGFP expression by >85% when tested using PD1 as the RSV sequence. The two VIROMIRs, RS001 and RS016 decreased GFP expression by 25% and 35% respectively in this experiment (FIG. 3). Using the PD2 construct, VIROMIRS RS026 and RS027 resulted in approximately 25% inhibition of GFP, while VIROMIR RS022 suppressed GFP fluorescence by approximately 10% (FIG. 4).

In some cases in this viral replication assay, an apparent stimulation of viral replication was observed which may represent a non-specific effect of the transfection process or presence of dsRNA.

VIROMIRs producing cytotoxicity or other undesirable effects may be excluded from use as a therapeutic. However, VIROMIRs that show apparent stimulation of RSV replication and/or IL-8 expression in a specific experimental system may be considered for further testing in other RSV disease models.

In this example, seeds common to both IL-8 and RSV were as follows: ACAAACUUUC (SEQ ID NO: 6), AACCAUCUCACU (SEQ ID NO: 7), CAUAAAGACAU (SEQ ID NO: 86), UUAUCAAAGAA (SEQ ID NO: 1), AUUGAAUGG, GAACUGAGA, GUGAUAUUUG (SEQ ID NO: 87), UGUGGUAUC, UCAAGCAAAU (SEQ ID NO: 88), CAGAUGCAA, AUACAAGAU, UUCCUGGUUA (SEQ ID NO: 89), AUCCAGAAC, AUAUAAGGAUU (SEQ ID NO: 90), UAGCAAAAUUG (SEQ ID NO: 91), CAUCAUAACA (SEQ ID NO: 92), AAUUUAGCUGGA (SEQ ID NO: 2), GGAAGCACU, AUAAAUUUCAA (SEQ ID NO: 93), CAUCAAAUAU (SEQ ID NO: 3), GAUUGAAUA, AUAGUUAUA, UUAUUAGAUAA (SEQ ID NO: 4), UUAGAUAAAU (SEQ ID NO: 94), AUUUCAAUCA (SEQ ID NO: 95), UUGAUACUCC (SEQ ID NO: 5), ACUAACAAU, UCCUAGUUU, AGUUUGAUAC (SEQ ID NO: 96), AUUGCCAGC, GAAUAAUGA, ACAGCCAAA, AUUAGUAAU, UUUAUUAUGU (SEQ ID NO: 97), CAAAUAGAU, AAUAGAUUC, AUAAUAUUAU (SEQ ID NO: 98), AUAUGAAAC, AGGACAAGA, UACAUUAUU and CUCUGUGGU.

It will be appreciated by one skilled in the art that multitargeting interfering RNA molecules (VIROMIRs) will comprise the sequence corresponding to the complement of the seed. In this example, these complementary sequences are: GAAAGUUUGU (SEQ ID NO: 112), AGUGAGAUGGUU (SEQ ID NO: 113), AUGUCUUUAUG (SEQ ID NO: 114), UUCUUUGAUAA (SEQ ID NO: 115), CCAUUCAAU, UCUCAGUUC, CAAAUAUCAC (SEQ ID NO: 116), GAUACCACA, AUUUGCUUGA (SEQ ID NO: 117), UUGCAUCUG, AUCUUGUAU, UAACCAGGAA (SEQ ID NO: 118), GUUCUGGAU, AAUCCUUAUAU (SEQ ID NO: 119), CAAUUUUGCUA (SEQ ID NO: 120), UGUUAUGAUG (SEQ ID NO: 121), UCCAGCUAAAUU (SEQ ID NO: 122), AGUGCUUCC, UUGAAAUUUAU (SEQ ID NO: 123), AUAUUUGAUG (SEQ ID NO: 124), UAUUCAAUC, UAUAACUAU, UUAUCUAAUAA (SEQ ID NO: 125), AUUUAUCUAA (SEQ ID NO: 126), UGAUUGAAAU (SEQ ID NO: 127), GGAGUAUCAA (SEQ ID NO: 128), AUUGUUAGU, AAACUAGGA, GUAUCAAACU (SEQ ID NO: 129), GCUGGCAAU, UCAUUAUUC, UUUGGCUGU, AUUACUAAU, ACAUAAUAAA (SEQ ID NO: 130), AUCUAUUUG, GAAUCUAUU, AUAAUAUUAU (SEQ ID NO: 98), GUUUCAUAU, UCUUGUCCU, AAUAAUGUA and ACCACAGAG.

It will be understood by one skilled in the art that these exemplary seeds, and their complete complements, also subsume any number of shorter seeds and their complete complements, respectively, and that these are envisaged as part of the invention. For example, the 10-base seed: ACAAACUUUC (SEQ ID NO: 6) comprises further two 9-base, three 8-base, four 7-base, and five 6-base seeds, all of which could be used in the design of useful multitargeting interfering RNA.

### Example 2

Targeting of Multiple Sites Within the RSV Genome

VIROMIRs can be used to target multiple sites in the genome of viruses. In this example, VIROMIRs were designed to target two sites in the Respiratory Syncytial Virus (RSV) genome. This approach has the advantage that in order for resistance to the VIROMIR to occur there would need to be at least 1 mutation at two different sites of the genome, thereby making the emergence of resistance less likely. The sequences from the RSV-GFP isolate used in the in vitro assays corresponding to the Accessory Phosphoprotein (P; similar to GenBank Accession Gene ID 1489821) and Large viral protein (L; similar to GenBank Accession Gene ID 1489827) were examined with bioinformatics methods to find seeds occurring at more than one location in the RSV genome.

The seeds that were identified as being at least 9 bases in length were: AAAGUUUGCU (SEQ ID NO: 99), AGAAGAUGC, AGAUAGUAU, UAUUGAUAC, AAAGAUCCCAA (SEQ ID NO: 100), AGUAUCAUA, UCAAUAGAUAUA (SEQ ID NO: 101), CCCUAUAACA (SEQ ID NO: 102), CAGAUGAUA, UAUCAUGUA, CUAAACUAUA (SEQ ID NO: 66), AAUCCAACA, AUCAACAUUGA (SEQ ID NO: 103), CGAUAAUAUAA (SEQ ID NO: 67), ACAUUAGUA, UGUAUAGCA, UAGAAGCUAU (SEQ ID NO: 104), UUUUUGUUCA (SEQ ID NO: 105), AUUGAACAACC (SEQ ID NO: 106), AUCAUCCAAC (SEQ ID NO: 107), UUGACUCAAU (SEQ ID NO: 108), UCAAGAUCU and AGAGGCUAU.

Two seeds selected for further evaluation were: CUAAACUAUA (SEQ ID NO: 66) and CGAUAAUAUAA (SEQ ID NO: 67). Both of the seeds are present in the sequences encoding the Accessory Phosphoprotein (P) and the Large viral protein (L). This is shown below with the bold regions corresponding to independent occurrences of the seed, along with the flanking sequences in the genes.

SEED 1:

(SEQ ID NO: 82)

5′ GACCCTACACCAAGTGATAATCCCTTTTCTAAACTATACAAAGAAAC
CATAGAAACAT 3′
Protein P

(SEQ ID NO: 83)

5′ TCATGTTTTAAATTTCAGATCAACAGAACTAAACTATAACCATTTAT
ATATGGTAGAA 3′
Protein L
SEED 2:

(SEQ ID NO: 84)

5′ TAAATGATCAGACAAACGATAATATAACAGCAAGATTAGATAGGAT
TGATGAAAAAT 3′
Protein P

(SEQ ID NO: 85)

5′ TAGATAAGACAGTGTCCGATAATATAATAAATGGCAGATGGATAAT
TCTATTAAGTA 3′
Protein L

Based on these seeds, 2 VIROMIRs were designed (Table 4). Consensus target sequences were designed for the two seeds. As appreciated by one skilled in the art there are many possible consensus target sequences, although only 1 such sequence in each case was used here. The guide strands were generated as the complements of these consensus target sequences as indicated above. The corresponding passenger strands were designed to be the complement of the guide strand, minus the first 2 bases at the 5′-extremity and with a 3′-extremity extension of UU, thereby generating dual 2-base overhangs at each 3′ extremity. Predicted binding of the guide strands of the two VIROMIRs to the RSV target sequence was determined using RNA hybrid to ensure appropriate binding (Table 5).

TABLE 4
List of VIROMIRs designed to target multiple sites in RSV

| | TARGET SEED | GUIDE STRAND | PASSENGER STRAND | DUPLEX (SEQ ID NOS |
|---|---|---|---|---|
| VIROMIR | (5′ to 3′) | (5′ TO 3′) | (5′ TO 3′) | 70, 68, 71 & 69) |
| RS014 | CUAAACUAUA | UAUAGUUUAGUUAAGGGAUUA | AUCCCUUAACUAAACUAUAUU | AUCCCUUAACUAAACUAUAUU |
| | (SEQ ID NO: 66) | (SEQ ID NO: 68) | (SEQ ID NO: 70) | AUUAGGGAAUUGAUUUGAUAU |
| RS015 | CGAUAAUAUAA | UUAUAUUAUCGAAUGUCUGAU | CAGACAUUCGAUAAUAUAAUU | CAGACAUUCGAUAAUAUAAUU |
| | (SEQ ID NO: 67) | (SEQ ID NO: 69) | (SEQ ID NO: 71) | UAGUCUGUAAGCUAUUAUAUU |

TABLE 5

Predicted binding of VIROMIRs (lower sequence)
targeting multiple sites in RSV (upper sequence).

| | | |
|---|---|---|
| VIROMIR | Target Binding Protein P (RNA Hybrid) | Target Binding Protein L (RNA Hybrid) |

| | | |
|---|---|---|
| RS014 | (SEQ ID NO: 206) | (SEQ ID NO: 204) |
| | target 5′ A UU C 3′ | target 5′ A AACAG A 3′ |
| | UAAUCCCUU CUAAACUAUA | GAUC AACUAAACUAUA |
| | AUUAGGGAA GAUUUGAUAU | UUAG UUGAUUUGAUAU |
| | 3′ UU 5′ | 3′ A GGAA 5′ |
| | (SEQ ID NO: 68) | (SEQ ID NO: 68) |
| RS015 | (SEQ ID NO: 190) | (SEQ ID NO: 205) |
| | target 5′ G AA C 3′ | target 5′ A GUGUC U 3′ |
| | AUCAGACA CGAUAAUAUAA | AGACA CGAUAAUAUAA |
| | UAGUCUGU GCUAUUAUAUU | UCUGU GCUAUUAUAUU |
| | 3′ AA 5′ | 3′ UAG AA 5′ |
| | (SEQ ID NO: 69) | (SEQ ID NO: 69) |

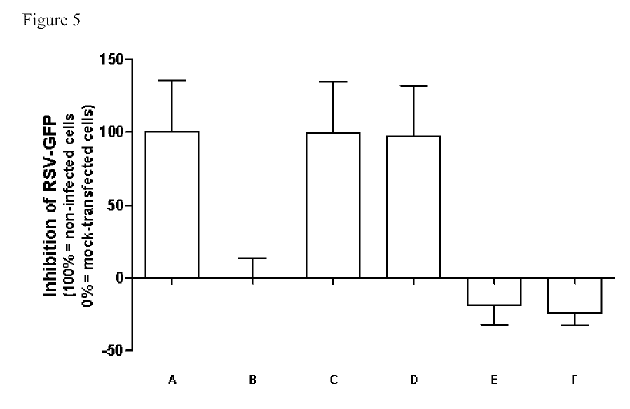

Each of the VIROMIRs was tested in two different RSV assays. The first assay utilized a GFP-tagged infectious RSV strain. The VIROMIRs were tested for their effect on RSV using A549 cells infected with the virus. A549 cells were plated on Day 1 in a 96 well plate at 4,000 cells/well (80 μL) in DMEM medium without antibiotics. The following day (Day 2), the cells were transfected with the VIROMIRs and control RSV siRNAs (siRSVP1 and siRSVP2; Table 6). For each well of a 96-well plate, 0.2 μL of a 20 μM stock of VIROMIR or siRNA was mixed with OptiMEM (10 μL final volume). This mixture was complexed for 20 minutes with 0.2 μL Lipofectamine2000 in 10 μL OptiMEM. The complex (20 μL) was added to cells in 80 μL DMEM medium so as to provide a final concentration of 40 nM dsRNA. Cells were infected on Day 3 by the addition of 100 μL of RSV-GFP to an MOI of 0.01. Two days later, fluorescence was measured, supernatants collected, cellular RNA extracted and RT-PCR performed on the human acidic ribosomal phosphoprotein PO (rplpo) transcript to evaluate toxicity. All treatments were performed in triplicate and the dsRNA were arranged across two plates with all positive (siRSVP1 and siRSVP2) and negative controls (untreated and mock-transfected cells) replicated on both plates. The sequences for the VIROMIRs and positive controls are shown in Tables 4 and 6, respectively. The positive control siRNAs for RSV (siRSVP1 and siRSVP2) suppressed RSV-GFP fluorescence by ˜100% (FIG. 5). The VIROMIRs RS014 and RS015 increased RSV replication by 18% and 24% respectively (FIG. 5).

TABLE 6
Sequences of siRNAs used as controls

| | | | |
|---|---|---|---|
| siRNA | GUIDE (5′ to 3′) | PASSENGER (5′ to 3′) | Reference |

| | | | |
|---|---|---|---|
| siRSVP1 | UCUUGCAGUUAUAUUAUCGdTdT | CGAUAAUAUAACAGCAAGAdTdT | Bitko, |
| | (SEQ ID NO: 72) | (SEQ ID NO: 76) | et al., |
| | | | 2005 |
| siRSVP2 | AUUAUCACUUGGUGUAGGGdTdT | CCCUACACCAAGUGAUAAUdTdT | Bitko, |
| | (SEQ ID NO: 73) | (SEQ ID NO: 77) | et al., |
| | | | 2005 |
| siGC47 | GUCUGCGAUCGCAUACAAUdTdT | AUUGUAUGCGAUCGCAGACdTdT | Dharma- |
| | (SEQ ID NO: 75) | (SEQ ID NO: 79) | con |

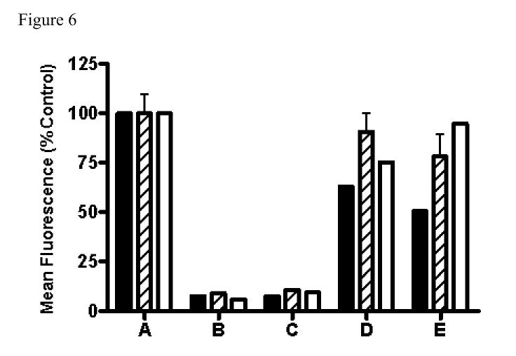

The second assay utilized an eGFP reporter-based system. A short RSV target sequence (181 nucleotides) encoding part of Accessory Phosphoprotein (P) was synthesized by Genscript Corporation (NJ, USA) and inserted into a GFP reporter plasmid (modified pd4-eGFP-N1 vector from Clontech). A549 cells in 6-well plates (1.2E+05 cells/well) were transfected as single samples with 300 ng or 750 ng plasmid PD1+300 ng RNA duplex or in triplicate with 500 ng plasmid+300 ng RNA duplex using 5 μL Lipofectamine 2000 in 250 μL OptiMEM in a total transfection volume of 2 mL. Fresh media was replaced 5 hours post-transfection and eGFP expression analysed by FACS 48 hours post-transfection. The two positive control siRNAs against RSV (siRSVP1 and siRSVP2) successfully inhibited eGFP expression by 90%. Both siRNAs have previously been shown to efficiently target the P mRNA of RSV and when administered intranasally, siRSVP1 worked to either treat or to prevent RSV infection in mice (Bitko, et al. 2005). In comparison, RS014 and RS015, which were tested using between 300 ng and 750 ng reporter plasmid, consistently showed inhibition of eGFP expression, with approximately 10-40% and 5-50% inhibition respectively (FIG. 6). RS014 and RS015 were designed to target both Protein P and Protein L. However, we note that the target sequence for Protein L is not included in this reporter construct, so inhibition by RS014 and RS015 may be lower than if the sequence for Protein L were included. Apparently discrepant results were obtained for RS014 and RS015 in the two assays. Further testing in other disease models may resolve this discrepancy.

It will be appreciated by one skilled in the art that multitargeting interfering RNA molecules (VIROMIRs) will comprise the sequence corresponding to the complement of the seed. In this example, these complementary sequences are: AGCAAACUUU (SEQ ID NO: 131), CGAUCUUCU, AUACUAUCU, GUAUCAAUA, UUGGGAUCUUU (SEQ ID NO: 132), UAUGAUACU, UAUAUCUAUUGA (SEQ ID NO: 133), UGUUAUAGGG (SEQ ID NO: 134), UAUCAUCUG, UACAUGAUA, UAUAGUUUAG (SEQ ID NO: 135), UGUUGGAUU, UCAAUGUUGAU (SEQ ID NO: 136), UUAUAUUAUCG (SEQ ID NO: 137), UACUAAUGU, UGCUAUACA, AUAGCUUCUA (SEQ ID NO: 138), UGAACAAAAA (SEQ ID NO: 139), GGUUGUUCAAU (SEQ ID NO: 140), GUUGGAUGAU (SEQ ID NO: 141), AUUGAGUCAA (SEQ ID NO: 142), AGAUCUUGA and AUAGCCUCU.

It will be understood by one skilled in the art that these exemplary seeds, and their complete complements, also subsume any number of shorter seeds and their complete complements, respectively, and that these are envisaged as part of the invention. For example, the 10-base seed: AGCAAACUUU (SEQ ID NO: 131) comprises further two 9-base, three 8-base, four 7-base, and five 6-base seeds, all of which could be used in the design of useful multitargeting interfering RNA.

### Example 3

### Targeting Multiple Viruses

In the setting of infections which are difficult to diagnose or which may occur as co-infections, it may be advantageous to have an active molecule capable of targeting several viruses simultaneously. For example, parainfluenza and RSV infections can present in similar circumstances and are difficult to differentiate. Examination of the parainfluenza 3 genome sequence (eg NCBI sequence Z11575) and that of the RSV phosphoprotein (NCBI M22644) revealed the presence of seeds common to both which may be exploited in the development of a multitargeting RNA of the invention which would be suitable for the treatment of either infection alone or as a co-infection. These include the following seeds:

| | |
|---|---|
| AGAAUCAAUAAAGG | (SEQ ID NO: 109) |
| AAAGAAGACCCUA | (SEQ ID NO: 110) |
| UGAUGAAAAAUU | (SEQ ID NO: 111) |

It will be appreciated by one skilled in the art that multitargeting interfering RNA molecules (e.g. VIROMIRs) will comprise the sequence corresponding to the complement of the seed. In this example, these complementary sequences are CCUUUAUUGAUUCU (SEQ ID NO: 143), UAGGGUCUUCUUU (SEQ ID NO: 144) and AAUUUUUCAUCA (SEQ ID NO: 145).

While the foregoing specification teaches the principles of the present invention, with examples provided for the purpose of illustration, it will be understood that the practice of the invention encompasses all of the usual variations, adaptations and/or modifications as come within the scope of the following claims and their equivalents. All references are hereby incorporated into this application in their entirety.

### REFERENCES

Bitko V, Musiyenko A, Shulyayeva O and Barik S. (2005). Inhibition of respiratory viruses by nasally administered siRNA. Nature Medicine 11:50-5.

Carillo et al, (1988), SIAM J. Applied Math. 48, 1073

Hossbach et al. (2006), RNA Biology 3: 82-89

Loakes, 2001, Nucleic Acids Research, 29, 2437-2447

Sambrook et al. (1989), Molecular Cloning: A Laboratory Manual, Second Edition, Cold Spring Harbor Laboratory, Cold Spring Harbor, N.Y.

Smyth, R. L., Mobbs, K. J., O'Hea, U., Ashby, D. & Hart, C. A. (2002). Respiratory syncytial virus bronchiolitis: disease severity, interleukin-8, and virus genotype. Pediatr Pulmonol, 33, 339-46.

Sugrue, R. (2006), Expert Rev Mol Med. 8:1-17.

1. A multitargeting interfering RNA molecule comprising a guide strand of Formula (I):
5′-p-XSY-3′
wherein p consists of a terminal phosphate group that is independently present or absent;
wherein S consists of a first nucleotide sequence of a length of about 5 to about 20 nucleotides that is at least partially complementary to a first portion of each of at least two binding sequences present in distinct genetic contexts in one or more pre-selected target RNA molecules;
wherein X is absent or consists of a second nucleotide sequence;
wherein Y is absent or consists of a third nucleotide sequence, provided that X and Y are not absent simultaneously;
wherein XSY is at least partially complementary to each of said binding sequences to allow a stable interaction therewith; and
wherein at least one of the binding sequences is present in paramyxovirus RNA or coronavirus, other than SARS, RNA.

2. The multitargeting interfering RNA molecule of claim 1, wherein S is completely complementary to the first portion of each of at least two binding sequences.

3. The multitargeting interfering RNA molecule of claim 1, wherein the first portion of each of at least two binding sequences is a seed sequence.

4. The multitargeting interfering RNA molecule of claim 1, wherein X consists of one or two nucleotides.

5. The multitargeting interfering RNA molecule of claim 1, wherein Y is at least partially complementary to a second portion of each of the binding sequences, said second portion is adjacent to and connected with the 5′-end of said first portion of the binding sequences.

6. The multitargeting interfering RNA molecule of claim 1, wherein S is of a length of about 8 to about 15 nucleotides.

7. The multitargeting interfering RNA molecule of claim 1, wherein XSY is of a length of about 17 to about 25 nucleotides.

8. The multitargeting interfering RNA molecule of claim 1, further comprising a passenger strand that is at least partially complementary to the guide strand to allow formation of a stable duplex between the passenger strand and the guide strand.

9. The multitargeting interfering RNA molecule of claim 8 comprising one or more terminal overhangs.

10. The multitargeting interfering RNA molecule of claim 9, wherein the overhang consists of 1 to 5 nucleotides.

11. The multitargeting interfering RNA molecule of claim 8, wherein the passenger strand and the guide strand, except for terminal overhangs, are completely complementary to each other.

12. The multitargeting interfering RNA molecule of claim 1, wherein the binding sequences are present in distinct genetic contexts in one pre-selected target RNA molecule.

13. The multitargeting interfering RNA molecule of claim 1, wherein the binding sequences are present in distinct genetic contexts in at least two pre-selected target RNA molecules.

14. The multitargeting interfering RNA molecule of claim 1, wherein the binding sequences are present in two different viruses.

15. The multitargeting interfering RNA molecule of claim 1, wherein at least one of the pre-selected target RNA molecules is a non-coding RNA molecule.

16. The multitargeting interfering RNA molecule of claim 1, wherein at least one of the pre-selected target RNA molecules is a messenger RNA molecule.

17. The multitargeting interfering RNA molecule of claim 1, wherein at least one of the binding sequences is present in the 3′-untranslated region (3′UTR) of a messenger RNA molecule.

18. The multitargeting interfering RNA molecule of claim 1, wherein the paramyxovirus is RSV or HPIV.

19. The multitargeting interfering RNA molecule of claim 1, wherein one or more of the pre-selected target RNA molecules encode a protein selected from the group consisting of IL-8, receptors to which RSV can bind such as heparan sulphate, GTP-binding proteins such as RhoA, cytoskeletal proteins such as actin, profilin and heat shock proteins such as Hsp70, cellular receptors such as angiotensin converting enzyme (ACE2), human aminopeptidase N, receptor glycoproteins and HLA class I antigens, proteins involved in signal transduction including MEK1/2 or ERF1/2, proteins involved in heparan sulfate synthesis such as heparan sulfate synthase, sialylglycoprotein cellular receptors, and protein synthesis and folding proteins such as Hsp90.

20. The multitargeting interfering RNA molecule of claim 1, wherein one or more of the pre-selected target RNA molecules encode IL-8.

21. The multitargeting interfering RNA molecule of claim 20 wherein one or more of the pre-selected RNA molecules comprises RSV RNA and one or more of the pre-selected RNA molecules encodes IL-8.

22. The multitargeting interfering RNA molecule of claim 1, wherein the molecule comprises a duplex selected from the group consisting of:

| | |
|---|---|
| 5′ CCCCAAUAUUAUCAAAGAAUU 3′ | (SEQ ID NO: 37) |
| 3′GUGGGGUUAUAAUAGUUUCUU 5′ | (SEQ ID NO: 8) |
| 5′ ACCCAUUCAGUGUGGUAUUUU 3′ | (SEQ ID NO: 39) |
| 3′UUUGGGUAAGUCACACCAUAG 5′ | (SEQ ID NO: 10) |
| 5′ GGUUCGCAGAUGCAACCAAUU 3′ | (SEQ ID NO: 40) |
| 3′UCCCAAGCGUCUACGUUGGUU 5′ | (SEQ ID NO: 11) |
| 5′ ACCAUGAAUAAUCCAGAAUUU 3′ | (SEQ ID NO: 41) |
| 3′CAUGGUACUUAUUAGGUCUUG5′ | (SEQ ID NO: 12) |
| 5′ CCAUGAAUAAUCCAGAAUAUU 3′ | (SEQ ID NO: 42) |
| 3′AUGGUACUUAUUAGGUCUUGU 5′ | (SEQ ID NO: 13) |
| 5′ GUCAAAUUUAGCUGGAAAUUU 3′ | (SEQ ID NO: 43) |
| 3′UUCAGUUUAAAUCGACCUUUA 5′ | (SEQ ID NO: 14) |
| 5′ CUUAUUUAUCCAUCAAAUAUU 3′ | (SEQ ID NO: 44) |
| 3′AUGAAUAAAUAGGUAGUUUAU 5′ | (SEQ ID NO: 15) |
| 5′ UGAUGAAUUAUUAGAUAAAUU 3′ | (SEQ ID NO: 46) |
| 3′UUACUACUUAAUAAUCUAUUU 5′ | (SEQ ID NO: 17) |
| 5′ UAGAUUUGAUACUCCUAAUUU 3′ | (SEQ ID NO: 47) |
| 3′CUAUCUAAACUAUGAGGGUUA 5′ | (SEQ ID NO: 18) |
| 5′ GAAUUAGCGAAUAAUGAAUUU 3′ | (SEQ ID NO: 48) |
| 3′AACUUAAUCGCUUAUUACUUA 5′ | (SEQ ID NO: 19) |
| 5′ CACAGUCAUAAUUAGUAAUUU 3′ | (SEQ ID NO: 49) |
| 3′AGGUGUCAGUAUUAAUCAUUA 5′ | (SEQ ID NO: 20) |
| 5′ GCCCAAAUUUAUCAAAGAAUU 3′ | (SEQ ID NO: 50) |
| 3′GUCGGGUUUAAAUAGUUUCUU 5′ | (SEQ ID NO: 21) |
| 5′ ACCCUAACCAUGUGGUAUUUU 3′ | (SEQ ID NO: 51) |
| 3′UUUGGGAUUGGUACACCAUAG 5′ | (SEQ ID NO: 22) |
| 5′ GUACAAUUUAGCUGGACAUUU 3′ | (SEQ ID NO: 52) |
| 3′UUCAUGUUAAAUCGACCUGUA 5′ | (SEQ ID NO: 23) |
| 5′ CUUCAAUAAACAUCAAAUAUU 3′ | (SEQ ID NO: 53) |
| 3′CAGAAGUUAUUUGUAGUUUAU 5′ | (SEQ ID NO: 24) |
| 5′ UCAUACAUUAUUAGAUAAAUU 3′ | (SEQ ID NO: 54) |
| 3′ACAGUAUGUAAUAAUCUAUUU 5′ | (SEQ ID NO: 25) |
| 5′ GCACAGCAACAUUAGUAAUUU 3′ | (SEQ ID NO: 55) |
| 3′UACGUGUCGUUGUAAUCAUUA 5′ | (SEQ ID NO: 26) |
| 5′ CUCCGAUUGAAUAGUUAUAUU 3′ | (SEQ ID NO: 56) |
| 3′UUGAGGCUAACUUAUCAAUAU 5′ | (SEQ ID NO: 27) |
| 5′ CACCUAGUUUAUAGUUAUAUU 3′ | (SEQ ID NO: 57) |
| 3′UAGUGGAUCAAAUAUCAAUAU 5′ | (SEQ ID NO: 28) |
| 5′ CCAAUAGACACAAACUUUCUU 3′ | (SEQ ID NO: 59) |
| 3′UCGGUUAUCUGUGUUUGAAAG 5′ | (SEQ ID NO: 30) |
| 5′ AUGAAGAAACCAUCUCACUUU 3′ | (SEQ ID NO: 60) |
| 3′AGUACUUCUUUGGUAGAGUGA 5′ | (SEQ ID NO: 31) |
| 5′ CGCUAUAAACCAUCUCACUUU 3′ | (SEQ ID NO: 61) |
| 3′UGGCGAUAUUUGGUAGAGUGA 5′ | (SEQ ID NO: 32) |
| 5′ ACAACCAACCCUCUGUGAUUU 3′ | (SEQ ID NO: 62) |
| 3′UUUGUUGGUUGGGAGACACCA 5′ | (SEQ ID NO: 33) |
| 5′ ACCACCCACCCUCUGUGAUUU 3′ | (SEQ ID NO: 63) |
| 3′AUUGGUGGGUGGGAGACACCA 5′ | (SEQ ID NO: 34) |
| 5′ CAACCAACCCUCUGUGGUUUU 3′ | (SEQ ID NO: 64) |
| 3′UUGUUGGUUGGGAGACACCAA 5′ | (SEQ ID NO: 35) |
| 5′ CCACCCACCCUCUGUGGUUUU 3′ | (SEQ ID NO: 65) |
| 3′UUGGUGGGUGGGAGACACCAA 5′ | (SEQ ID NO: 36) |
| 5′ AUCCCUUAACUAAACUAUAUU 3′ | (SEQ ID NO: 70) |
| 3′AUUAGGGAAUUGAUUUGAUAU 5′ | (SEQ ID NO: 68) |
| 5′ CAGACAUUCGAUAAUAUAAUU 3′ | (SEQ ID NO: 71) |
| 3′UAGUCUGUAAGCUAUUAUAUU 5′ | (SEQ ID NO: 69) |

23. The multitargeting interfering RNA molecule of claim 1 comprising at least one modified ribonucleotide or analogue, universal base, acyclic nucleotide, abasic nucleotide, non-ribonucleotide or combinations thereof.

24. (canceled)

25. (canceled)

26. A vector comprising a nucleotide sequence that encodes the multitargeting interfering RNA molecule of claim 1.

27. The vector of claim 26 being a viral vector.

28. The vector of claim 26 that is derived from a virus selected from the group consisting of an adeno-associated virus, a retrovirus, an adenovirus, a lentivirus, and an alphavirus.

29. A cell comprising the vector of claim 26.

30. The multitargeting interfering RNA molecule of claim 1 produced from a short hairpin RNA molecule.

31. (canceled)

32. (canceled)

33. A pharmaceutical composition comprising a multitargeting interfering RNA molecule of claim 1 and an acceptable carrier.

34. (canceled)

35. (canceled)

36. A method of inducing RNA interference in a biological system, comprising the step of introducing a multitargeting interfering RNA molecule of claim 1 into the biological system.

37. A method of inducing RNA interference in a biological system, comprising the steps of:
(a) selecting one or more target RNA molecules;
(b) designing a multitargeting interfering RNA molecule comprising a guide strand that can form stable interactions with at least two binding sequences present in distinct genetic contexts in the set of one or more target RNA molecules, wherein at least one of the binding sequences is present in paramyxovirus RNA or coronavirus, other than SARS, RNA.
(c) producing the multitargeting interfering RNA molecule; and
(d) administering the multitargeting interfering RNA molecule into the biological system, whereby the guide strand of the multitargeting interfering RNA molecule forms stable interactions with the binding sequences present in distinct genetic contexts in the target RNA molecules, and thus induces RNA interference of the target RNA molecules.

38. (canceled)

39. (canceled)

40. (canceled)

41. The method of claim 37, wherein the one or more target RNA molecules comprise a RNA molecule encoding a protein selected from the group consisting of of IL-8, receptors to which RSV can bind such as heparan sulphate, GTP-binding proteins such as RhoA, cytoskeletal proteins such as actin, profilin and heat shock proteins such as Hsp70, cellular receptors such as angiotensin converting enzyme (ACE2), human aminopeptidase N, receptor glycoproteins and HLA class I antigens, proteins involved in signal transduction including MEK1/2 or ERF1/2, proteins involved in heparan sulfate synthesis such as heparan sulfate synthase, sialylglycoprotein cellular receptors, and protein synthesis and folding proteins such as Hsp90.

42. (canceled)

43. (canceled)

44. (canceled)

45. (canceled)

46. (canceled)

47. (canceled)

48. (canceled)

49. A multitargeting interfering RNA molecule comprising Formula (II):
5′-p-A B C-3′
3′-A′B′C′-p-5′
wherein p consists of a terminal phosphate group that is independently present or absent; wherein B consists of a first nucleotide sequence of a length of about 5 to about 20 nucleotides that is partially, preferably completely, complementary to a first portion of a first binding sequence, and B′ consists of a second nucleotide sequence of a length of about 5 to about 20 nucleotides that is partially, preferably completely, complementary to a first portion of a second binding sequence, wherein said first and second binding sequences are present in distinct genetic contexts in at least one pre-selected target RNA molecule, and wherein B and B′ are at least substantially complementary to each other but are not palindromic; and further wherein A, A′, C, or C′, is independently absent or consists of a nucleotide sequence; wherein ABC is at least partially complementary to the first binding sequence to allow stable interaction therewith; and wherein C′B′A′ is at least partially complementary to the second binding sequence to allow stable interaction therewith and is at least partially complementary to ABC to form a stable duplex therewith and wherein at least one of the first or second binding sequences is present in paramyxovirus RNA or coronavirus, other than SARS, RNA.

50. The multitargeting interfering RNA molecule of claim 49, wherein A, A′, C, or C′, independently consists of one or more nucleotides.

51. The multitargeting interfering RNA molecule of claim 49, wherein A consists of a third nucleotide sequence that is at least partially complementary to a second portion of the first binding sequence, said second portion is adjacent to and connected with the 3′-end of said first portion of the first binding sequence, and wherein A′ consists of a fourth nucleotide sequence that is substantially complementary to the third nucleotide sequence.

52. The multitargeting interfering RNA molecule of claim 49, wherein A and A′ are completely complementary to each other.

53. The multitargeting interfering RNA molecule of claim 49, wherein A is completely complementary to the second portion of the first binding sequence.

54. The multitargeting interfering RNA molecule of claim 49, wherein C′ consists of a fifth nucleotide sequence that is at least partially complementary to a second portion of the second binding sequence, said second portion is adjacent to and connected with the 3′-end of said first portion of the second binding sequence, and wherein C consists of a sixth nucleotide sequence that is substantially complementary to the fifth nucleotide sequence.

55. The multitargeting interfering RNA molecule of claim 49, wherein C and C′ are completely complementary to each other.

56. The multitargeting interfering RNA molecule of claim 49, wherein C′ is completely complementary to the second portion of the second binding sequence.

57. The multitargeting interfering RNA molecule of claim 49, wherein B and B′ are completely complementary to each other.

58. The multitargeting interfering RNA molecule of claim 49, wherein AB is completely complementary to the first portion and the second portion of the first binding sequence.

59. The multitargeting interfering RNA molecule of claim 49, wherein C′B′ is completely complementary to the first portion and the second portion of the second binding sequence.

60. (canceled)

61. (canceled)

62. (canceled)

63. (canceled)

64. (canceled)

65. (canceled)

66. (canceled)

67. The multitargeting interfering RNA molecule of claim 49 comprising at least one 2′-O-methyl ribosyl substitution or a locked nucleic acid ribonucleotide.

68. (canceled)

69. (canceled)

70. (canceled)

71. (canceled)

72. (canceled)

73. The multitargeting interfering RNA molecule of claim 49, wherein at least one of the binding sequences is present in the 3′-untranslated region (3′UTR) of a mRNA molecule.

74. (canceled)

75. (canceled)

76. (canceled)

77. (canceled)

78. (canceled)

79. (canceled)

80. (canceled)

81. (canceled)

82. (canceled)

83. The multitargeting interfering RNA molecule of claim 49 wherein the molecule is a short hairpin RNA molecule.

84. (canceled)

85. (canceled)

86. (canceled)

87. (canceled)

88. (canceled)

89. (canceled)

90. A method of treating a subject, comprising the step of administering to said subject a therapeutically effective amount of a pharmaceutical composition comprising a multitargeting interfering RNA molecule of claim 49.

91. The method of claim 90, further comprising administering to said subject a therapeutically effective amount of one or more additional therapeutic agents.

92. (canceled)

93. (canceled)

94. (canceled)

95. A multitargeting interfering RNA molecule comprising a sequence selected from the group consisting of:
(SEQ ID NO: 6)
ACAAACUUUC,
(SEQ ID NO: 7)
AACCAUCUCACU,
(SEQ ID NO: 86)
CAUAAAGACAU,
(SEQ ID NO: 1)
UUAUCAAAGAA,
(SEQ ID NO: 87)
AUUGAAUGG, GAACUGAGA, GUGAUAUUUG,
(SEQ ID NO: 88)
UGUGGUAUC, UCAAGCAAAU,
(SEQ ID NO: 89)
CAGAUGCAA, AUACAAGAU, UUCCUGGUUA,
(SEQ ID NO: 90)
AUCCAGAAC, AUAUAAGGAUU,
(SEQ ID NO: 91)
UAGCAAAAUUG,
(SEQ ID NO: 92)
CAUCAUAACA,
(SEQ ID NO: 2)
AAUUUAGCUGGA,
(SEQ ID NO: 93)
GGAAGCACU, AUAAAUUUCAA,
(SEQ ID NO: 3)
CAUCAAAUAU,
(SEQ ID NO: 4)
GAUUGAAUA, AUAGUUAUA, UUAUUAGAUAA,
(SEQ ID NO: 94)
UUAGAUAAAU,
(SEQ ID NO: 95)
AUUUCAAUCA,
(SEQ ID NO: 5)
UUGAUACUCC,
(SEQ ID NO: 96)
ACUAACAAU, UCCUAGUUU, AGUUUGAUAC,
(SEQ ID NO: 97)
AUUGCCAGC, GAAUAAUGA, ACAGCCAAA, AUUAGUAAU,
UUUAUUAUGU,
(SEQ ID NO: 98)
CAAAUAGAU, AAUAGAUUC, AUAAUAUUAU,
(SEQ ID NO: 99)
AUAUGAAAC, AGGACAAGA, UACAUUAUU, CUCUGUGGU,
AAAGUUUGCU,
(SEQ ID NO: 100)
AGAAGAUGC, AGAUAGUAU, UAUUGAUAC, AAAGAUCCCAA,
(SEQ ID NO: 101)
AGUAUCAUA, UCAAUAGAUAUA,
(SEQ ID NO: 102)
CCCUAUAACA,
(SEQ ID NO: 66)
CAGAUGAUA, UAUCAUGUA, CUAAACUAUA,
(SEQ ID NO: 103)
AAUCCAACA, AUCAACAUUGA,
(SEQ ID NO: 67)
CGAUAAUAUAA,
(SEQ ID NO: 104)
ACAUUAGUA, UGUAUAGCA, UAGAAGCUAU,
(SEQ ID NO: 105)
UUUUUGUUCA,
(SEQ ID NO: 106)
AUUGAACAACC,
(SEQ ID NO: 107)
AUCAUCCAAC,
(SEQ ID NO: 108)
UUGACUCAAU,
(SEQ ID NO: 109)
UCAAGAUCU, AGAGGCUAU, AGAAUCAAUAAAGG,
(SEQ ID NO: 110)
AAAGAAGACCCUA,
(SEQ ID NO: 111)
UGAUGAAAAAUU,
(SEQ ID NO: 112)
GAAAGUUUGU,
(SEQ ID NO: 113)
AGUGAGAUGGUU,
(SEQ ID NO: 114)
AUGUCUUUAUG,
(SEQ ID NO: 115)
UUCUUUGAUAA,
(SEQ ID NO: 116)
CCAUUCAAU, UCUCAGUUC, CAAAUAUCAC,
(SEQ ID NO: 117)
GAUACCACA, AUUUGCUUGA,
(SEQ ID NO: 118)
UUGCAUCUG, AUCUUGUAU, UAACCAGGAA,
(SEQ ID NO: 119)
GUUCUGGAU, AAUCCUUAUAU,
(SEQ ID NO: 120)
CAAUUUUGCUA,
(SEQ ID NO: 121)
UGUUAUGAUG,
(SEQ ID NO: 122)
UCCAGCUAAAUU,
(SEQ ID NO: 123)
AGUGCUUCC, UUGAAAUUUAU,
(SEQ ID NO: 124)
AUAUUUGAUG,
(SEQ ID NO: 125)
UAUUCAAUC, UAUAACUAU, UUAUCUAAUAA,
(SEQ ID NO: 126)
AUUUAUCUAA,
(SEQ ID NO: 127)
UGAUUGAAAU,
(SEQ ID NO: 128)
GGAGUAUCAA,
(SEQ ID NO: 129)
AUUGUUAGU, AAACUAGGA, GUAUCAAACU,
(SEQ ID NO: 130)
GCUGGCAAU, UCAUUAUUC, UUUGGCUGU, AUUACUAAU,
ACAUAAUAAA,
(SEQ ID NO: 98)
AUCUAUUUG, GAAUCUAUU, AUAAUAUUAU,
(SEQ ID NO: 131)
GUUUCAUAU, UCUUGUCCU, AAUAAUGUA, ACCACAGAG,
AGCAAACUUU,
(SEQ ID NO: 132)
CGAUCUUCU, AUACUAUCU, GUAUCAAUA, UUGGGAUCUUU,
(SEQ ID NO: 133)
UAUGAUACU, UAUAUCUAUUGA,
(SEQ ID NO: 134)
UGUUAUAGGG,
(SEQ ID NO: 135)
UAUCAUCUG, UACAUGAUA, UAUAGUUUAG,
(SEQ ID NO: 136)
UGUUGGAUU, UCAAUGUUGAU,
(SEQ ID NO: 137)
UUAUAUUAUCG,
(SEQ ID NO: 138)
UACUAAUGU, UGCUAUACA, AUAGCUUCUA,
(SEQ ID NO: 139)
UGAACAAAAA,
(SEQ ID NO: 140)
GGUUGUUCAAU,
(SEQ ID NO: 141)
GUUGGAUGAU,
(SEQ ID NO: 142)
AUUGAGUCAA,
(SEQ ID NO: 143)
AGAUCUUGA, AUAGCCUCU, CCUUUAUUGAUUCU,
(SEQ ID NO: 144)
UAGGGUCUUCUUU
and
(SEQ ID NO: 145)
AAUUUUUCAUCA.

96. A method for introducing a multitargeting interfering RNA molecule comprising Formula (II) or a guide strand of Formula (I) into a cell comprising the steps of:
i) generating a multitargeting interfering RNA molecule comprising Formula (I) or Formula (II) and;
ii) contacting the multitargeting interfering RNA molecule comprising Formula (I) or Formula (II) with a cell,
wherein Formula (I) is
5′-p-XSY-3′
wherein p consists of a terminal phosphate group that is independently present or absent;
wherein S consists of a first nucleotide sequence of a length of about 5 to about 20 nucleotides that is at least partially complementary to a first portion of each of at least two binding sequences present in distinct genetic contexts in one or more pre-selected target RNA molecules;
wherein X is absent or consists of a second nucleotide sequence;
wherein Y is absent or consists of a third nucleotide sequence, provided that X and Y are not absent simultaneously;
wherein XSY is at least partially complementary to each of said binding sequences to allow a stable interaction therewith; and
wherein at least one of the binding sequences is present in paramyxovirus RNA or coronavirus, other than SARS, RNA,
and
Formula (II) is
5′-p-A B C-3′
3′-A′B′C′-p-5′
wherein p consists of a terminal phosphate group that is independently present or absent; wherein B consists of a first nucleotide sequence of a length of about 5 to about 20 nucleotides that is partially, preferably completely, complementary to a first portion of a first binding sequence, and B′ consists of a second nucleotide sequence of a length of about 5 to about 20 nucleotides that is partially, preferably completely, complementary to a first portion of a second binding sequence, wherein said first and second binding sequences are present in distinct genetic contexts in at least one pre-selected target RNA molecule, and wherein B and B′ are at least substantially complementary to each other but are not palindromic; and further wherein A, A′, C, or C′, is independently absent or consists of a nucleotide sequence; wherein ABC is at least partially complementary to the first binding sequence to allow stable interaction therewith; and wherein C′B′A′ is at least partially complementary to the second binding sequence to allow stable interaction therewith and is at least partially complementary to ABC to form a stable duplex therewith and wherein at least one of the first or second binding sequences is present in paramyxovirus RNA or coronavirus, other than SARS, RNA.

97. The method of claim 96 wherein the multitargeting interfering RNA is encoded by DNA.

98. (canceled)

99. The method of claim 96 wherein the contacting step further comprises the step of introducing the RNA molecule into the cell.


SEQUENCE LISTING (206 sequences)

SEQ ID NO: 1 - RNA, 11 nt, Artificial Sequence
  Description of Artificial Sequence Synthetic oligonucleotide
uuaucaaagaa

SEQ ID NO: 2 - RNA, 12 nt, Artificial Sequence
  Description of Artificial Sequence Synthetic oligonucleotide
aauuuagcugga

SEQ ID NO: 3 - RNA, 10 nt, Artificial Sequence
  Description of Artificial Sequence Synthetic oligonucleotide
caucaaauau

SEQ ID NO: 4 - RNA, 11 nt, Artificial Sequence
  Description of Artificial Sequence Synthetic oligonucleotide
uuauuagauaa

SEQ ID NO: 5 - RNA, 10 nt, Artificial Sequence
  Description of Artificial Sequence Synthetic oligonucleotide
uugauacucc

SEQ ID NO: 6 - RNA, 10 nt, Artificial Sequence
  Description of Artificial Sequence Synthetic oligonucleotide
acaaacuuuc

SEQ ID NO: 7 - RNA, 12 nt, Artificial Sequence
  Description of Artificial Sequence Synthetic oligonucleotide
aaccaucucacu

SEQ ID NO: 8 - RNA, 21 nt, Artificial Sequence
  Description of Artificial Sequence Synthetic oligonucleotide
uucuuugauaauauuggggug

SEQ ID NO: 9 - RNA, 21 nt, Artificial Sequence
  Description of Artificial Sequence Synthetic oligonucleotide
ucucaguucaaugauagguuu

SEQ ID NO: 10 - RNA, 21 nt, Artificial Sequence
  Description of Artificial Sequence Synthetic oligonucleotide
gauaccacacugaauggguuu

SEQ ID NO: 11 - RNA, 21 nt, Artificial Sequence
  Description of Artificial Sequence Synthetic oligonucleotide
uugguugcaucugcgaacccu

SEQ ID NO: 12 - RNA, 21 nt, Artificial Sequence
  Description of Artificial Sequence Synthetic oligonucleotide
guucuggauuauucaugguac

SEQ ID NO: 13 - RNA, 21 nt, Artificial Sequence
  Description of Artificial Sequence Synthetic oligonucleotide
uguucuggauuauucauggua

SEQ ID NO: 14 - RNA, 21 nt, Artificial Sequence
  Description of Artificial Sequence Synthetic oligonucleotide
auuuccagcuaaauuugacuu

SEQ ID NO: 15 - RNA, 21 nt, Artificial Sequence
  Description of Artificial Sequence Synthetic oligonucleotide
uauuugauggauaaauaagua

SEQ ID NO: 16 - RNA, 21 nt, Artificial Sequence
  Description of Artificial Sequence Synthetic oligonucleotide
uuauucaaucuuugauucauc

SEQ ID NO: 17 - RNA, 21 nt, Artificial Sequence
  Description of Artificial Sequence Synthetic oligonucleotide
uuuaucuaauaauucaucauu

SEQ ID NO: 18 - RNA, 21 nt, Artificial Sequence
  Description of Artificial Sequence Synthetic oligonucleotide
auugggaguaucaaaucuauc

SEQ ID NO: 19 - RNA, 21 nt, Artificial Sequence
  Description of Artificial Sequence Synthetic oligonucleotide
auucauuauucgcuaauucaa

SEQ ID NO: 20 - RNA, 21 nt, Artificial Sequence
  Description of Artificial Sequence Synthetic oligonucleotide
auuacuaauuaugacugugga

SEQ ID NO: 21 - RNA, 21 nt, Artificial Sequence
  Description of Artificial Sequence Synthetic oligonucleotide
uucuuugauaaauuugggcug

SEQ ID NO: 22 - RNA, 21 nt, Artificial Sequence
  Description of Artificial Sequence Synthetic oligonucleotide
gauaccacaugguuaggguuu

SEQ ID NO: 23 - RNA, 21 nt, Artificial Sequence
  Description of Artificial Sequence Synthetic oligonucleotide
auguccagcuaaauuguacuu

SEQ ID NO: 24 - RNA, 21 nt, Artificial Sequence
  Description of Artificial Sequence Synthetic oligonucleotide
uauuugauguuuauugaagac

SEQ ID NO: 25 - RNA, 21 nt, Artificial Sequence
  Description of Artificial Sequence Synthetic oligonucleotide
uuuaucuaauaauguaugaca

SEQ ID NO: 26 - RNA, 21 nt, Artificial Sequence
  Description of Artificial Sequence Synthetic oligonucleotide
auuacuaauguugcugugcau

SEQ ID NO: 27 - RNA, 21 nt, Artificial Sequence
  Description of Artificial Sequence Synthetic oligonucleotide
uauaacuauucaaucggaguu

SEQ ID NO: 28 - RNA, 21 nt, Artificial Sequence
  Description of Artificial Sequence Synthetic oligonucleotide
uauaacuauaaacuaggugau

SEQ ID NO: 29 - RNA, 21 nt, Artificial Sequence
  Description of Artificial Sequence Synthetic oligonucleotide
gaaaguuugugugauauggag

SEQ ID NO: 30 - RNA, 21 nt, Artificial Sequence
  Description of Artificial Sequence Synthetic oligonucleotide
gaaaguuugugucuauuggcu

SEQ ID NO: 31 - RNA, 21 nt, Artificial Sequence
  Description of Artificial Sequence Synthetic oligonucleotide
agugagaugguuucuucauga

SEQ ID NO: 32 - RNA, 21 nt, Artificial Sequence
  Description of Artificial Sequence Synthetic oligonucleotide
agugagaugguuuauagcggu

SEQ ID NO: 33 - RNA, 21 nt, Artificial Sequence
  Description of Artificial Sequence Synthetic oligonucleotide
accacagaggguugguuguuu

SEQ ID NO: 34 - RNA, 21 nt, Artificial Sequence
  Description of Artificial Sequence Synthetic oligonucleotide
accacagagggugggugguua

SEQ ID NO: 35 - RNA, 21 nt, Artificial Sequence
  Description of Artificial Sequence Synthetic oligonucleotide
aaccacagaggguugguuguu

SEQ ID NO: 36 - RNA, 21 nt, Artificial Sequence
  Description of Artificial Sequence Synthetic oligonucleotide
aaccacagagggugggugguu

SEQ ID NO: 37 - RNA, 21 nt, Artificial Sequence
  Description of Artificial Sequence Synthetic oligonucleotide
ccccaauauuaucaaagaauu

SEQ ID NO: 38 - RNA, 21 nt, Artificial Sequence
  Description of Artificial Sequence Synthetic oligonucleotide
accuaucauugaacugagauu

SEQ ID NO: 39 - RNA, 21 nt, Artificial Sequence
  Description of Artificial Sequence Synthetic oligonucleotide
acccauucagugugguauuuu

SEQ ID NO: 40 - RNA, 21 nt, Artificial Sequence
  Description of Artificial Sequence Synthetic oligonucleotide
gguucgcagaugcaaccaauu

SEQ ID NO: 41 - RNA, 21 nt, Artificial Sequence
  Description of Artificial Sequence Synthetic oligonucleotide
accaugaauaauccagaauuu

SEQ ID NO: 42 - RNA, 21 nt, Artificial Sequence
  Description of Artificial Sequence Synthetic oligonucleotide
ccaugaauaauccagaauauu

SEQ ID NO: 43 - RNA, 21 nt, Artificial Sequence
  Description of Artificial Sequence Synthetic oligonucleotide
gucaaauuuagcuggaaauuu

SEQ ID NO: 44 - RNA, 21 nt, Artificial Sequence
  Description of Artificial Sequence Synthetic oligonucleotide
cuuauuuauccaucaaauauu

SEQ ID NO: 45 - RNA, 21 nt, Artificial Sequence
  Description of Artificial Sequence Synthetic oligonucleotide
ugaaucaaagauugaauaauu

SEQ ID NO: 46 - RNA, 21 nt, Artificial Sequence
  Description of Artificial Sequence Synthetic oligonucleotide
ugaugaauuauuagauaaauu

SEQ ID NO: 47 - RNA, 21 nt, Artificial Sequence
  Description of Artificial Sequence Synthetic oligonucleotide
uagauuugauacuccuaauuu

SEQ ID NO: 48 - RNA, 21 nt, Artificial Sequence
  Description of Artificial Sequence Synthetic oligonucleotide
gaauuagcgaauaaugaauuu

SEQ ID NO: 49 - RNA, 21 nt, Artificial Sequence
  Description of Artificial Sequence Synthetic oligonucleotide
cacagucauaauuaguaauuu

SEQ ID NO: 50 - RNA, 21 nt, Artificial Sequence
  Description of Artificial Sequence Synthetic oligonucleotide
gcccaaauuuaucaaagaauu

SEQ ID NO: 51 - RNA, 21 nt, Artificial Sequence
  Description of Artificial Sequence Synthetic oligonucleotide
acccuaaccaugugguauuuu

SEQ ID NO: 52 - RNA, 21 nt, Artificial Sequence
  Description of Artificial Sequence Synthetic oligonucleotide
guacaauuuagcuggacauuu

SEQ ID NO: 53 - RNA, 21 nt, Artificial Sequence
  Description of Artificial Sequence Synthetic oligonucleotide
cuucaauaaacaucaaauauu

SEQ ID NO: 54 - RNA, 21 nt, Artificial Sequence
  Description of Artificial Sequence Synthetic oligonucleotide
ucauacauuauuagauaaauu

SEQ ID NO: 55 - RNA, 21 nt, Artificial Sequence
  Description of Artificial Sequence Synthetic oligonucleotide
gcacagcaacauuaguaauuu

SEQ ID NO: 56 - RNA, 21 nt, Artificial Sequence
  Description of Artificial Sequence Synthetic oligonucleotide
cuccgauugaauaguuauauu

SEQ ID NO: 57 - RNA, 21 nt, Artificial Sequence
  Description of Artificial Sequence Synthetic oligonucleotide
caccuaguuuauaguuauauu

SEQ ID NO: 58 - RNA, 21 nt, Artificial Sequence
  Description of Artificial Sequence Synthetic oligonucleotide
ccauaucacacaaacuuucuu

SEQ ID NO: 59 - RNA, 21 nt, Artificial Sequence
  Description of Artificial Sequence Synthetic oligonucleotide
ccaauagacacaaacuuucuu

SEQ ID NO: 60 - RNA, 21 nt, Artificial Sequence
  Description of Artificial Sequence Synthetic oligonucleotide
augaagaaaccaucucacuuu

SEQ ID NO: 61 - RNA, 21 nt, Artificial Sequence
  Description of Artificial Sequence Synthetic oligonucleotide
cgcuauaaaccaucucacuuu

SEQ ID NO: 62 - RNA, 21 nt, Artificial Sequence
  Description of Artificial Sequence Synthetic oligonucleotide
acaaccaacccucugugauuu

SEQ ID NO: 63 - RNA, 21 nt, Artificial Sequence
  Description of Artificial Sequence Synthetic oligonucleotide
accacccacccucugugauuu

SEQ ID NO: 64 - RNA, 21 nt, Artificial Sequence
  Description of Artificial Sequence Synthetic oligonucleotide
caaccaacccucugugguuuu

SEQ ID NO: 65 - RNA, 21 nt, Artificial Sequence
  Description of Artificial Sequence Synthetic oligonucleotide
ccacccacccucugugguuuu

SEQ ID NO: 66 - RNA, 10 nt, Artificial Sequence
  Description of Artificial Sequence Synthetic oligonucleotide
cuaaacuaua

SEQ ID NO: 67 - RNA, 11 nt, Artificial Sequence
  Description of Artificial Sequence Synthetic oligonucleotide
cgauaauauaa

SEQ ID NO: 68 - RNA, 21 nt, Artificial Sequence
  Description of Artificial Sequence Synthetic oligonucleotide
uauaguuuaguuaagggauua

SEQ ID NO: 69 - RNA, 21 nt, Artificial Sequence
  Description of Artificial Sequence Synthetic oligonucleotide
uuauauuaucgaaugucugau

SEQ ID NO: 70 - RNA, 21 nt, Artificial Sequence
  Description of Artificial Sequence Synthetic oligonucleotide
aucccuuaacuaaacuauauu

SEQ ID NO: 71 - RNA, 21 nt, Artificial Sequence
  Description of Artificial Sequence Synthetic oligonucleotide
cagacauucgauaauauaauu

SEQ ID NO: 72 - DNA, 21 bp, Artificial Sequence
  Description of DNA/RNA hybrid molecule Synthetic oligonucleotide
ucuugcaguuauauuaucgtt

SEQ ID NO: 73 - DNA, 21 bp, Artificial Sequence
  Description of DNA/RNA hybrid molecule Synthetic oligonucleotide
auuaucacuugguguagggtt

SEQ ID NO: 74 - DNA, 21 bp, Artificial Sequence
  Description of DNA/RNA hybrid molecule Synthetic oligonucleotide
gaugguuccuuccgguggutt

SEQ ID NO: 75 - DNA, 21 bp, Artificial Sequence
  Description of DNA/RNA hybrid molecule Synthetic oligonucleotide
gucugcgaucgcauacaautt

SEQ ID NO: 76 - DNA, 21 bp, Artificial Sequence
  Description of DNA/RNA hybrid molecule Synthetic oligonucleotide
cgauaauauaacagcaagatt

SEQ ID NO: 77 - DNA, 21 bp, Artificial Sequence
  Description of DNA/RNA hybrid molecule Synthetic oligonucleotide
cccuacaccaagugauaautt

SEQ ID NO: 78 - DNA, 21 bp, Artificial Sequence
  Description of DNA/RNA hybrid molecule Synthetic oligonucleotide
accaccggaaggaaccauctt

SEQ ID NO: 79 - DNA, 21 bp, Artificial Sequence
  Description of DNA/RNA hybrid molecule Synthetic oligonucleotide
auuguaugcgaucgcagactt

SEQ ID NO: 80 - DNA, 208 bp, Artificial Sequence
  Description of Artificial Sequence Synthetic nucleotide construct
ggctcgagtgattgattgaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattccgcggtt

SEQ ID NO: 81 - DNA, 191 bp, Artificial Sequence
  Description of Artificial Sequence Synthetic nucleotide construct
ggctcgagtgattgattgaataaaaatagcacctaatcatgttcttacaatggtttactatctgctcatagacaacccatctgtcattggattttcttaaaatctgaacttcatcgaaactctcatctataaaccatctcacttacactatttaagtagattcctagtttatagttatataaaccgcggtt

SEQ ID NO: 82 - DNA, 58 bp, Artificial Sequence
  Description of Artificial Sequence Synthetic polynucleotide
gaccctacaccaagtgataatcccttttctaaactatacaaagaaaccatagaaacat

SEQ ID NO: 83 - DNA, 58 bp, Artificial Sequence
  Description of Artificial Sequence Synthetic polynucleotide
tcatgttttaaatttcagatcaacagaactaaactataaccatttatatatggtagaa

SEQ ID NO: 84 - DNA, 57 bp, Artificial Sequence
  Description of Artificial Sequence Synthetic polynucleotide
taaatgatcagacaaacgataatataacagcaagattagataggattgatgaaaaat

SEQ ID NO: 85 - DNA, 57 bp, Artificial Sequence
  Description of Artificial Sequence Synthetic polynucleotide
tagataagacagtgtccgataatataataaatggcagatggataattctattaagta

SEQ ID NO: 86 - RNA, 11 nt, Artificial Sequence
  Description of Artificial Sequence Synthetic oligonucleotide
cauaaagacau

SEQ ID NO: 87 - RNA, 10 nt, Artificial Sequence
  Description of Artificial Sequence Synthetic oligonucleotide
gugauauuug

SEQ ID NO: 88 - RNA, 10 nt, Artificial Sequence
  Description of Artificial Sequence Synthetic oligonucleotide
ucaagcaaau

SEQ ID NO: 89 - RNA, 10 nt, Artificial Sequence
  Description of Artificial Sequence Synthetic oligonucleotide
uuccugguua

SEQ ID NO: 90 - RNA, 11 nt, Artificial Sequence
  Description of Artificial Sequence Synthetic oligonucleotide
auauaaggauu

SEQ ID NO: 91 - RNA, 11 nt, Artificial Sequence
  Description of Artificial Sequence Synthetic oligonucleotide
uagcaaaauug

SEQ ID NO: 92 - RNA, 10 nt, Artificial Sequence
  Description of Artificial Sequence Synthetic oligonucleotide
caucauaaca

SEQ ID NO: 93 - RNA, 11 nt, Artificial Sequence
  Description of Artificial Sequence Synthetic oligonucleotide
auaaauuucaa

SEQ ID NO: 94 - RNA, 10 nt, Artificial Sequence
  Description of Artificial Sequence Synthetic oligonucleotide
uuagauaaau

SEQ ID NO: 95 - RNA, 10 nt, Artificial Sequence
  Description of Artificial Sequence Synthetic oligonucleotide
auuucaauca

SEQ ID NO: 96 - RNA, 10 nt, Artificial Sequence
  Description of Artificial Sequence Synthetic oligonucleotide
aguuugauac

SEQ ID NO: 97 - RNA, 10 nt, Artificial Sequence
  Description of Artificial Sequence Synthetic oligonucleotide
uuuauuaugu

SEQ ID NO: 98 - RNA, 10 nt, Artificial Sequence
  Description of Artificial Sequence Synthetic oligonucleotide
auaauauuau

SEQ ID NO: 99 - RNA, 10 nt, Artificial Sequence
  Description of Artificial Sequence Synthetic oligonucleotide
aaaguuugcu

SEQ ID NO: 100 - RNA, 11 nt, Artificial Sequence
  Description of Artificial Sequence Synthetic oligonucleotide
aaagaucccaa

SEQ ID NO: 101 - RNA, 12 nt, Artificial Sequence
  Description of Artificial Sequence Synthetic oligonucleotide
ucaauagauaua

SEQ ID NO: 102 - RNA, 10 nt, Artificial Sequence
  Description of Artificial Sequence Synthetic oligonucleotide
cccuauaaca

SEQ ID NO: 103 - RNA, 11 nt, Artificial Sequence
  Description of Artificial Sequence Synthetic oligonucleotide
aucaacauuga

SEQ ID NO: 104 - RNA, 10 nt, Artificial Sequence
  Description of Artificial Sequence Synthetic oligonucleotide
uagaagcuau

SEQ ID NO: 105 - RNA, 10 nt, Artificial Sequence
  Description of Artificial Sequence Synthetic oligonucleotide
uuuuuguuca

SEQ ID NO: 106 - RNA, 11 nt, Artificial Sequence
  Description of Artificial Sequence Synthetic oligonucleotide
auugaacaacc

SEQ ID NO: 107 - RNA, 10 nt, Artificial Sequence
  Description of Artificial Sequence Synthetic oligonucleotide
aucauccaac

SEQ ID NO: 108 - RNA, 10 nt, Artificial Sequence
  Description of Artificial Sequence Synthetic oligonucleotide
uugacucaau

SEQ ID NO: 109 - RNA, 14 nt, Artificial Sequence
  Description of Artificial Sequence Synthetic oligonucleotide
agaaucaauaaagg

SEQ ID NO: 110 - RNA, 13 nt, Artificial Sequence
  Description of Artificial Sequence Synthetic oligonucleotide
aaagaagacccua

SEQ ID NO: 111 - RNA, 12 nt, Artificial Sequence
  Description of Artificial Sequence Synthetic oligonucleotide
ugaugaaaaauu

SEQ ID NO: 112 - RNA, 10 nt, Artificial Sequence
  Description of Artificial Sequence Synthetic oligonucleotide
gaaaguuugu

SEQ ID NO: 113 - RNA, 12 nt, Artificial Sequence
  Description of Artificial Sequence Synthetic oligonucleotide
agugagaugguu

SEQ ID NO: 114 - RNA, 11 nt, Artificial Sequence
  Description of Artificial Sequence Synthetic oligonucleotide
augucuuuaug

SEQ ID NO: 115 - RNA, 11 nt, Artificial Sequence
  Description of Artificial Sequence Synthetic oligonucleotide
uucuuugauaa

SEQ ID NO: 116 - RNA, 10 nt, Artificial Sequence
  Description of Artificial Sequence Synthetic oligonucleotide
caaauaucac

SEQ ID NO: 117 - RNA, 10 nt, Artificial Sequence
  Description of Artificial Sequence Synthetic oligonucleotide
auuugcuuga

SEQ ID NO: 118 - RNA, 10 nt, Artificial Sequence
  Description of Artificial Sequence Synthetic oligonucleotide
uaaccaggaa

SEQ ID NO: 119 - RNA, 11 nt, Artificial Sequence
  Description of Artificial Sequence Synthetic oligonucleotide
aauccuuauau

SEQ ID NO: 120 - RNA, 11 nt, Artificial Sequence
  Description of Artificial Sequence Synthetic oligonucleotide
caauuuugcua

SEQ ID NO: 121 - RNA, 10 nt, Artificial Sequence
  Description of Artificial Sequence Synthetic oligonucleotide
uguuaugaug

SEQ ID NO: 122 - RNA, 12 nt, Artificial Sequence
  Description of Artificial Sequence Synthetic oligonucleotide
uccagcuaaauu

SEQ ID NO: 123 - RNA, 11 nt, Artificial Sequence
  Description of Artificial Sequence Synthetic oligonucleotide
uugaaauuuau

SEQ ID NO: 124 - RNA, 10 nt, Artificial Sequence
  Description of Artificial Sequence Synthetic oligonucleotide
auauuugaug

SEQ ID NO: 125 - RNA, 11 nt, Artificial Sequence
  Description of Artificial Sequence Synthetic oligonucleotide
uuaucuaauaa

SEQ ID NO: 126 - RNA, 10 nt, Artificial Sequence
  Description of Artificial Sequence Synthetic oligonucleotide
auuuaucuaa

SEQ ID NO: 127 - RNA, 10 nt, Artificial Sequence
  Description of Artificial Sequence Synthetic oligonucleotide
ugauugaaau

SEQ ID NO: 128 - RNA, 10 nt, Artificial Sequence
  Description of Artificial Sequence Synthetic oligonucleotide
ggaguaucaa

SEQ ID NO: 129 - RNA, 10 nt, Artificial Sequence
  Description of Artificial Sequence Synthetic oligonucleotide
guaucaaacu

SEQ ID NO: 130 - RNA, 10 nt, Artificial Sequence
  Description of Artificial Sequence Synthetic oligonucleotide
acauaauaaa

SEQ ID NO: 131 - RNA, 10 nt, Artificial Sequence
  Description of Artificial Sequence Synthetic oligonucleotide
agcaaacuuu

SEQ ID NO: 132 - RNA, 11 nt, Artificial Sequence
  Description of Artificial Sequence Synthetic oligonucleotide
uugggaucuuu

SEQ ID NO: 133 - RNA, 12 nt, Artificial Sequence
  Description of Artificial Sequence Synthetic oligonucleotide
uauaucuauuga

SEQ ID NO: 134 - RNA, 10 nt, Artificial Sequence
  Description of Artificial Sequence Synthetic oligonucleotide
uguuauaggg

SEQ ID NO: 135 - RNA, 10 nt, Artificial Sequence
  Description of Artificial Sequence Synthetic oligonucleotide
uauaguuuag

SEQ ID NO: 136 - RNA, 11 nt, Artificial Sequence
  Description of Artificial Sequence Synthetic oligonucleotide
ucaauguugau

SEQ ID NO: 137 - RNA, 11 nt, Artificial Sequence
  Description of Artificial Sequence Synthetic oligonucleotide
uuauauuaucg

SEQ ID NO: 138 - RNA, 10 nt, Artificial Sequence
  Description of Artificial Sequence Synthetic oligonucleotide
auagcuucua

SEQ ID NO: 139 - RNA, 10 nt, Artificial Sequence
  Description of Artificial Sequence Synthetic oligonucleotide
ugaacaaaaa

SEQ ID NO: 140 - RNA, 11 nt, Artificial Sequence
  Description of Artificial Sequence Synthetic oligonucleotide
gguuguucaau

SEQ ID NO: 141 - RNA, 10 nt, Artificial Sequence
  Description of Artificial Sequence Synthetic oligonucleotide
guuggaugau

SEQ ID NO: 142 - RNA, 10 nt, Artificial Sequence
  Description of Artificial Sequence Synthetic oligonucleotide
auugagucaa

SEQ ID NO: 143 - RNA, 14 nt, Artificial Sequence
  Description of Artificial Sequence Synthetic oligonucleotide
ccuuuauugauucu

SEQ ID NO: 144 - RNA, 13 nt, Artificial Sequence
  Description of Artificial Sequence Synthetic oligonucleotide
uagggucuucuuu

SEQ ID NO: 145 - RNA, 12 nt, Artificial Sequence
  Description of Artificial Sequence Synthetic oligonucleotide
aauuuuucauca

SEQ ID NO: 146 - DNA, 10 bp, Artificial Sequence
  Description of Artificial Sequence Synthetic oligonucleotide
ctgacaatcg

SEQ ID NO: 147 - DNA, 10 bp, Artificial Sequence
  Description of Artificial Sequence Synthetic oligonucleotide
cgaaagtcag

SEQ ID NO: 148 - DNA, 10 bp, Artificial Sequence
  Description of Artificial Sequence Synthetic oligonucleotide
cgattgtcag

SEQ ID NO: 149 - RNA, 20 nt, Artificial Sequence
  Description of Artificial Sequence Synthetic oligonucleotide
aagcccaauuaucaaagaaa

SEQ ID NO: 150 - RNA, 28 nt, Artificial Sequence
  Description of Artificial Sequence Synthetic oligonucleotide
uaggccacaaauuaaaaggaacugagac

SEQ ID NO: 151 - RNA, 22 nt, Artificial Sequence
  Description of Artificial Sequence Synthetic oligonucleotide
caacccuaaucaugugguauca

SEQ ID NO: 152 - RNA, 15 nt, Artificial Sequence
  Description of Artificial Sequence Synthetic oligonucleotide
acagaugcaaccaac

SEQ ID NO: 153 - RNA, 22 nt, Artificial Sequence
  Description of Artificial Sequence Synthetic oligonucleotide
cuaccacaggaaauccagaacu

SEQ ID NO: 154 - RNA, 21 nt, Artificial Sequence
  Description of Artificial Sequence Synthetic oligonucleotide
caccacaggaaauccagaacu

SEQ ID NO: 155 - RNA, 21 nt, Artificial Sequence
  Description of Artificial Sequence Synthetic oligonucleotide
uaaguacuaauuuagcuggac

SEQ ID NO: 156 - RNA, 11 nt, Artificial Sequence
  Description of Artificial Sequence Synthetic oligonucleotide
acaucaaauau

SEQ ID NO: 157 - RNA, 19 nt, Artificial Sequence
  Description of Artificial Sequence Synthetic oligonucleotide
agaugauagauugaauaag

SEQ ID NO: 158 - RNA, 24 nt, Artificial Sequence
  Description of Artificial Sequence Synthetic oligonucleotide
uaugucauacauuauuagauaaga

SEQ ID NO: 159 - RNA, 23 nt, Artificial Sequence
  Description of Artificial Sequence Synthetic oligonucleotide
agauaguauugauacuccuaauu

SEQ ID NO: 160 - RNA, 14 nt, Artificial Sequence
  Description of Artificial Sequence Synthetic oligonucleotide
augaauaaugaaua

SEQ ID NO: 161 - RNA, 20 nt, Artificial Sequence
  Description of Artificial Sequence Synthetic oligonucleotide
guauagcagcauuaguaaua

SEQ ID NO: 162 - RNA, 22 nt, Artificial Sequence
  Description of Artificial Sequence Synthetic oligonucleotide
caacccuaaucaugugguaucu

SEQ ID NO: 163 - RNA, 23 nt, Artificial Sequence
  Description of Artificial Sequence Synthetic oligonucleotide
ccaccccaaauuuaucaaagaac

SEQ ID NO: 164 - RNA, 23 nt, Artificial Sequence
  Description of Artificial Sequence Synthetic oligonucleotide
caaauuuaucaaagaacugagag

SEQ ID NO: 165 - RNA, 23 nt, Artificial Sequence
  Description of Artificial Sequence Synthetic oligonucleotide
aaaauucauucucugugguaucc

SEQ ID NO: 166 - RNA, 24 nt, Artificial Sequence
  Description of Artificial Sequence Synthetic oligonucleotide
uaggguugccagaugcaauacaag

SEQ ID NO: 167 - RNA, 23 nt, Artificial Sequence
  Description of Artificial Sequence Synthetic oligonucleotide
uguaccaugaaauauccagaaca

SEQ ID NO: 168 - RNA, 23 nt, Artificial Sequence
  Description of Artificial Sequence Synthetic oligonucleotide
guaccaugaaauauccagaacau

SEQ ID NO: 169 - RNA, 23 nt, Artificial Sequence
  Description of Artificial Sequence Synthetic oligonucleotide
aaagucaaauuuagcuggaaauc

SEQ ID NO: 170 - RNA, 23 nt, Artificial Sequence
  Description of Artificial Sequence Synthetic oligonucleotide
guauuuauuuaagcaucaaauau

SEQ ID NO: 171 - RNA, 23 nt, Artificial Sequence
  Description of Artificial Sequence Synthetic oligonucleotide
agaugaaucauugauugaauagu

SEQ ID NO: 172 - RNA, 23 nt, Artificial Sequence
  Description of Artificial Sequence Synthetic oligonucleotide
aaaugauguuuuauuagauaaau

SEQ ID NO: 173 - RNA, 20 nt, Artificial Sequence
  Description of Artificial Sequence Synthetic oligonucleotide
cuaguuugauacucccaguc

SEQ ID NO: 174 - RNA, 23 nt, Artificial Sequence
  Description of Artificial Sequence Synthetic oligonucleotide
guugaauuacggaauaaugaguu

SEQ ID NO: 175 - RNA, 23 nt, Artificial Sequence
  Description of Artificial Sequence Synthetic oligonucleotide
cuccacagucaauauuaguaauu

SEQ ID NO: 176 - RNA, 24 nt, Artificial Sequence
  Description of Artificial Sequence Synthetic oligonucleotide
uaaguacuaauuuagcuggacauu

SEQ ID NO: 177 - RNA, 18 nt, Artificial Sequence
  Description of Artificial Sequence Synthetic oligonucleotide
acaauggacaucaaauau

SEQ ID NO: 178 - RNA, 23 nt, Artificial Sequence
  Description of Artificial Sequence Synthetic oligonucleotide
augucauacauuauuagauaaga

SEQ ID NO: 179 - RNA, 23 nt, Artificial Sequence
  Description of Artificial Sequence Synthetic oligonucleotide
uauguauagcagcauuaguaaua

SEQ ID NO: 180 - RNA, 23 nt, Artificial Sequence
  Description of Artificial Sequence Synthetic oligonucleotide
agauuccuaguuuauaguuauau

SEQ ID NO: 181 - RNA, 19 nt, Artificial Sequence
  Description of Artificial Sequence Synthetic oligonucleotide
uccuaguuuauaguuauau

SEQ ID NO: 182 - RNA, 21 nt, Artificial Sequence
  Description of Artificial Sequence Synthetic oligonucleotide
guuaauucauacaaacuuucu

SEQ ID NO: 183 - RNA, 22 nt, Artificial Sequence
  Description of Artificial Sequence Synthetic oligonucleotide
uguuaauucauacaaacuuucu

SEQ ID NO: 184 - RNA, 23 nt, Artificial Sequence
  Description of Artificial Sequence Synthetic oligonucleotide
cucaucuauaaaccaucucacuu

SEQ ID NO: 185 - RNA, 27 nt, Artificial Sequence
  Description of Artificial Sequence Synthetic oligonucleotide
aacucucaucuauaaaccaucucacuu

SEQ ID NO: 186 - RNA, 24 nt, Artificial Sequence
  Description of Artificial Sequence Synthetic oligonucleotide
cacaaucacaaacacucugugguu

SEQ ID NO: 187 - RNA, 22 nt, Artificial Sequence
  Description of Artificial Sequence Synthetic oligonucleotide
caaucacaaacacucugugguu

SEQ ID NO: 188 - RNA, 25 nt, Artificial Sequence
  Description of Artificial Sequence Synthetic oligonucleotide
cacaaucacaaacacucugugguuc

SEQ ID NO: 189 - RNA, 23 nt, Artificial Sequence
  Description of Artificial Sequence Synthetic oligonucleotide
caaucacaaacacucugugguuc

SEQ ID NO: 190 - RNA, 23 nt, Artificial Sequence
  Description of Artificial Sequence Synthetic oligonucleotide
gaucagacaaacgauaauauaac

SEQ ID NO: 191 - RNA, 20 nt, Artificial Sequence
  Description of Artificial Sequence Synthetic oligonucleotide
aaagucaaauuuagcuggaa

SEQ ID NO: 192 - RNA, 21 nt, Artificial Sequence
  Description of Artificial Sequence Synthetic oligonucleotide
auuuauuuaagcaucaaauau

SEQ ID NO: 193 - RNA, 18 nt, Artificial Sequence
  Description of Artificial Sequence Synthetic oligonucleotide
auguuuuauuagauaaau

SEQ ID NO: 194 - RNA, 21 nt, Artificial Sequence
  Description of Artificial Sequence Synthetic oligonucleotide
ccacagucaauauuaguaauu

SEQ ID NO: 195 - RNA, 19 nt, Artificial Sequence
  Description of Artificial Sequence Synthetic oligonucleotide
auugauugaauaguuauaa

SEQ ID NO: 196 - RNA, 23 nt, Artificial Sequence
  Description of Artificial Sequence Synthetic oligonucleotide
aaucauugauugaauaguuauaa

SEQ ID NO: 197 - RNA, 22 nt, Artificial Sequence
  Description of Artificial Sequence Synthetic oligonucleotide
cuccauaaggcacaaacuuuca

SEQ ID NO: 198 - RNA, 21 nt, Artificial Sequence
  Description of Artificial Sequence Synthetic oligonucleotide
uccauaaggcacaaacuuuca

SEQ ID NO: 199 - RNA, 22 nt, Artificial Sequence
  Description of Artificial Sequence Synthetic oligonucleotide
accggaaggaaccaucucacug

SEQ ID NO: 200 - RNA, 23 nt, Artificial Sequence
  Description of Artificial Sequence Synthetic oligonucleotide
caccggaaggaaccaucucacug

SEQ ID NO: 201 - RNA, 19 nt, Artificial Sequence
  Description of Artificial Sequence Synthetic oligonucleotide
aaauucauucucuguggua

SEQ ID NO: 202 - RNA, 22 nt, Artificial Sequence
  Description of Artificial Sequence Synthetic oligonucleotide
aaaaaauucauucucuguggua

SEQ ID NO: 203 - RNA, 18 nt, Artificial Sequence
  Description of Artificial Sequence Synthetic oligonucleotide
aauucauucucuguggua

SEQ ID NO: 204 - RNA, 23 nt, Artificial Sequence
  Description of Artificial Sequence Synthetic oligonucleotide
agaucaacagaacuaaacuauaa

SEQ ID NO: 205 - RNA, 23 nt, Artificial Sequence
  Description of Artificial Sequence Synthetic oligonucleotide
aagacaguguccgauaauauaau

SEQ ID NO: 206 - RNA, 23 nt, Artificial Sequence
  Description of Artificial Sequence Synthetic oligonucleotide
auaaucccuuuucuaaacuauac